(19) United States
(12) Patent Application Publication
(10) Pub. No.: US 2024/0131198 A1
(43) Pub. Date: Apr. 25, 2024

(54) RATIOMETRIC BIOSENSOR TATTOOS AND USE THEREOF FOR REAL TIME MEASUREMENTS

(71) Applicants: Davide Brambilla, Montreal (CA); Samuel Babity, Westmount (CA)

(72) Inventors: Davide Brambilla, Montreal (CA); Samuel Babity, Westmount (CA)

(21) Appl. No.: 18/225,564

(22) Filed: Jul. 24, 2023

(30) Foreign Application Priority Data
Jul. 23, 2022 (CA) 3168590

(51) Int. Cl. A61K 49/00; A61M 37/00

(52) U.S. Cl. CPC A61K 49/0032; A61K 49/0006; A61K 49/0054; A61M 37/0015; A61M 2037/0061

(57) ABSTRACT

The present disclosure relates to ratiometric detection compositions comprising a reference dye and a sensor dye that are PEGylated dyes, and microneedles comprising said compositions. The present disclosure further relates to methods of ratiometric detection/measurement/monitoring of analytes in a subject using the ratiometric detection compositions of the present disclosure.

### CROSS-REFERENCE TO RELATED APPLICATIONS

The present disclosure claims the benefit of priority from Canadian patent application no. 3,168,590, filed Jul. 23, 2022, the contents of which are incorporated herein by reference in their entirety.

### FIELD

The present disclosure relates to a ratiometric detection/measurement/monitoring composition comprising a reference dye and a sensor dye comprising an analyte sensing moiety and microneedles comprising said compositions. The present disclosure further relates to methods of ratiometric detection/measurement/monitoring of an analyte in a subject using the ratiometric detection composition of the present disclosure. Also included are kits comprising the composition of the present disclosure.

### INTRODUCTION

In recent years, there has been a growing interest in precision health monitoring, particularly in the context of early disease detection and preventive medicine. Though most medical diagnosis takes place after the presentation of symptoms, earlier detection and preventive monitoring could allow for more effective treatment.^{[1]} While this sort of monitoring is an attractive concept, its utility has historically been limited by the invasive nature of implantable devices or blood sampling, and the limited parameters that can be measured at the skin's surface. This has led to research into less invasive forms of biosensing, with many examples proposing optical, fluorescence-based techniques for direct in vivo imaging.^{[2,3]}

To this end, other monitoring targets have also been proposed, notably dermal interstitial fluid (ISF): the extracellular fluid surrounding cells in the dermal and epidermal layers of the skin. Recent studies have suggested that there is an overlap of roughly 93% between the proteomes expressed in plasma and in the dermal ISF.^{[4]} Additionally, similar populations of small molecules and ions have been observed, with alterations in plasma levels rapidly reflected in the ISF, suggesting that this fluid could provide an alternative to blood-based monitoring for many biological analytes.^{[5-8]} Among the advantages of ISF-based monitoring is ease-of-access; owing to its proximity to the surface of the skin, beginning in many regions of the body at depths of less than 30 μm (directly beneath the stratum corneum), this fluid could be reached using relatively non-invasive techniques.

One such technique involves the use of microneedles (MNs): miniaturized needles with lengths below 1 mm, often organized into an array.^{[9]} Short enough to avoid activating pain-sensing neurons found deeper in the dermis, MNs can nonetheless easily breach the stratum corneum, providing a means of accessing the ISF. Studies have attempted to exploit this feature, using hollow or swellable polymeric MNs to withdraw ISF through the skin for external analysis.^{[8,10,11]} Among these, experiments have been conducted using closed-loop insulin delivery systems, integrating glucose-sensing and insulin delivery elements into a single MN array, highlighting the versatility of polymeric MN technology.^{[12-14]} Similar techniques have also seen commercial application, notably including the FreeStyle Libre® glucose monitoring system, consisting of a continuously worn 5-mm long filament attached to a sensing device, which can be read using a portable detector.

However, these methods have limitations, including the cost of hollow MN-based devices, and the low collection volumes and requirement for post-collection analysis associated with swellable polymeric MNs.^{[15]} This has led to interest in an alternative approach, namely the use of dissolving polymeric MNs for the delivery of sensors directly to the dermis, allowing instantaneous in situ analysis.^{[9]} These soluble MNs, often made from polysaccharides or biocompatible synthetic polymers, have advantages, including simple, solvent-casting based manufacturing from inexpensive and readily available materials, and the generation of no sharp waste after use.^{[16]} Early examples of this approach have been proposed for the imaging and quantification of lymphatic drainage in the context of detecting structural modification involved in diseases such as cancer and lymphedema, as well as for monitoring vaccination status.^{[17-20]}

Potential applications in the skin have been limited, however, by their relatively poor water solubility and poor measurement accuracy, as for example topological variability of the skin's surface might lead to variability in administration, difference in chemical structure of the dyes could lead to different pharmacokinetic properties such as distribution and excretion, and consequently in fluorescent signal.^{[21,42]}

Real time monitoring of chronic diseases for example remains a challenge. [Biosensors (Basel). 2021 September; 11(9): 296]. One field that still needs attention is the efficacy of in vivo sampling with MN-based sensors. [Biosensors (Basel). 2021 September; 11(9): 296].

Accordingly, there is a need for development of better real time sensors for measurement of analytes.

### SUMMARY

Accordingly, herein is provided a transient ratiometric microneedle tattoo, controlling for the variable amount delivered, and allowing the fluorescent microneedle tattoo to provide ratiometric sensing and monitoring. Ultimately, this acts as a universal approach for the normalization of functional microneedle tattoos and has been applied to the quantification of any physiological analyte through this method. It has been shown that a dissolving polymeric MN-based delivery system for PEGylated hydrocyanines, capable of ratiometric detection and imaging of skin inflammation can be used for detection, measurement, and/or monitoring of various analytes including ROS, pH, and cation such as Na⁺. Notably, while the fluorescence of the microneedle tattoo is readily measured, it remains entirely invisible to the naked eye after application. Also shown herein is a multi-wavelength portable monitoring system for the in vivo detection of a ratiometric fluorescent microneedle tattoo in the skin, using a custom-built fluorescent monitoring device.^{[18,19]} This is an example of a functional fluorescent medical microneedle tattoo delivered using a minimally invasive MN system, as well as a novel approach to ratiometric monitoring. Existing ratiometric fluorescent sensors are limited, often displaying low-wavelength fluorescence and poor biocompatibility, preventing their use in the skin,^{[43-46]} and the present approach for a ratiometric microneedle tattoo allows for the sensing and monitoring of a variety of conditions.

It has been shown that an inert fluorescent dye with similar properties to the fluorescent sensor (but a different emission wavelength) loaded into the MNs and delivered alongside the sensor acts as a reference independent of analyte concentration, resulting in a microtattoo displaying a ratiometric fluorescent signal. Without wishing to be bound by theory, functionalization of the inert fluorescent dye and of the sensor dye with PEG of similar size minimizes differences in physical and chemical properties between the two dyes, thus making the pharmacokinetic properties of the dyes more similar. This allows correction for the variable quantity of sensor delivered by the MNs between different applications due to the non-homogeneous nature of the skin's surface, and facilitates formulation of the dyes for example into MNs. Further, the exemplary polymers used to prepare the MNs used for this technology (e.g. ultra-low molecular weight hyaluronic acid (MW<6,000 Da) and dextran (MW=6,000 Da)) are pharmaceutically inert and/or approved for injection. In some examples, the fluorescent sensors and reference dyes used (cyanines) share the same core structure as indocyanine green (ICG), an approved, injectable fluorescent contrast agent. This, coupled with the extremely small quantity of polymer and sensor delivered to the skin (in the low microgram range),^{[1]} facilitate clinical and commercial translation of the technology.

By delivering these sensors directly to the epidermis or dermis, MNs could be used to create a physiologically responsive medical microneedle tattoo.

Reactive oxygen species (ROS) are highly reactive compounds involved in multiple biological processes.^{[2-24]} ROS are also associated with disease, and elevated levels can lead to oxidative stress, resulting in DNA damage, cell death, and are characteristic of a variety of pathological conditions, including disorders of the skin. As a prominent example, exposure to UV radiation has been demonstrated to induce the production of dermal ROS.^{[31]}

Notably, the role of ROS in the development of atopic dermatitis has also been under investigation, owing to the increasing prevalence of this disease.^{[31,32]} Interestingly, atopic dermatitis is associated with elevated levels of markers of oxidative stress and lipid peroxidation, as well as decreased systemic levels of antioxidants (including glutathione, bilirubin, and vitamin C), indicating that ROS potentially drive the disease to a certain extent.^{[33-37]} In light of this, there has been interest in the monitoring of ROS in the skin to better understand such conditions and to evaluate their progression and treatment.^{[38]} ROS can be used for fluorescent sensing, as a some fluorescent dyes display little to no fluorescence in their reduced states, but display bright fluorescence in their oxidized forms. Hydrocyanines are a class of bright, easily prepared dyes displaying specific sensitivity to superoxide and hydroxyl radicals, two of the most biologically relevant ROS.^{[39]}

In one aspect, the present disclosure includes a ratiometric detection composition comprising
a reference dye, the reference dye being a first cyanine dye that is PEGylated; and
a sensor dye, the sensor dye being a second cyanine dye that is PEGylated and that comprises an analyte sensing moiety;
wherein the reference dye is inert towards the analyte;
wherein an emission maximum wavelength of the reference dye and an emission maximum wavelength of the sensor dye are at least about 50 nm apart;
wherein the first cyanine dye and the second cyanine dye are PEGylated with PEG groups of substantially the same molecular weight.

In another aspect, the present disclosure includes a reference dye compound as defined in the present disclosure.

In another aspect, the present disclosure includes a sensor dye compound as defined in the present disclosure.

In another aspect, the present disclosure includes a microneedle array comprising
a backing layer, and
a plurality of microneedles each having a first end and a second sharpened end for penetration into skin, the plurality of microneedles extending outwardly from the backing layer at the first end;
wherein the microneedles are formed with a biodegradable polymer and comprise a ratiometric detection composition of the present disclosure that is incorporated in the biodegradable polymer;
wherein the plurality of microneedles is dissolvable and releasable from the backing layer after penetration into the skin.

In another aspect, the present disclosure includes a method of detection of an analyte comprising
detecting in a subject that had been administered a ratiometric detection composition of the present disclosure:
   a fluorescence level of the reference dye; and
   a fluorescence level of the sensor dye;
assessing a presence of the analyte, wherein a positive value of the fluorescence level of the sensor dye normalized to the fluorescence level of the reference dye is indicative of the presence of the analyte.

In another aspect, the present disclosure includes a method of measurement of an analyte comprising
detecting in a subject that had been administered a ratiometric detection composition of the present disclosure:
   a fluorescence level of the reference dye; and
   a fluorescence level of the sensor dye; and
determining a quantity of the analyte, wherein a value of the fluorescence level of the sensor dye normalized to the fluorescence level of the reference dye is indicative of the quantity of the analyte.

In another aspect, the present disclosure includes a method for monitoring of an analyte comprising
a) detecting in a subject that had been administered a ratiometric detection composition of the present disclosure:
   an initial fluorescence level of the reference dye at an initial time point; and
   an initial fluorescence level of the sensor dye at the initial time point;
b) determining a quantity of the analyte at the initial time point, wherein a value of the initial fluorescence level of the sensor dye normalized to the initial fluorescence level of the reference dye is indicative of the quantity of the analyte at the initial time point;
c) detecting:
   a subsequent fluorescence level of the reference dye at a subsequent time point; and
   a subsequent fluorescence level of the sensor dye at the subsequent time point;
d) determining a quantity of the analyte at the subsequent time point, wherein a value of the subsequent fluorescence level of the sensor dye normalized to the subsequent fluorescence level of the reference dye is indicative of the quantity of the analyte at the subsequent time point; and
e) optionally repeating steps c) to d).

In another aspect, the present disclosure includes a method of detection of ROS comprising
detecting in a subject that had been administered a ratiometric detection composition of the present disclosure:
   a fluorescence level of the reference dye; and
   a fluorescence level of the sensor dye; and
assessing a presence of the ROS, wherein a positive value of the fluorescence level of the sensor dye normalized to the fluorescence level of the reference dye is indicative of the presence of the ROS.

In another aspect, the present disclosure includes a method of measurement of ROS comprising
detecting in a subject that had been administered a ratiometric detection composition of the present disclosure:
   a fluorescence level of the reference dye; and
   a fluorescence level of the sensor dye; and
determining a quantity of the ROS, wherein a value of the fluorescence level of the sensor dye normalized to the fluorescence level of the reference dye is indicative of the quantity of the ROS.

In another aspect, the present disclosure includes a method for monitoring of ROS comprising
a) detecting in a subject that had been administered a ratiometric detection composition of the present disclosure:
   an initial fluorescence level of the reference dye at an initial time point; and
   an initial fluorescence level of the sensor dye at the initial time point;
b) determining a quantity of the analyte at the initial time point, wherein a value of the initial fluorescence level of the sensor dye normalized to the initial fluorescence level of the reference dye is indicative of the quantity of the ROS at the initial time point;
c) detecting in the subject:
   a subsequent fluorescence level of the reference dye at a subsequent time point; and
   a subsequent fluorescence level of the sensor dye at the subsequent time point; and
d) determining a quantity of the analyte at the subsequent time point, wherein a value of the subsequent fluorescence level of the sensor dye normalized to the subsequent fluorescence level of the reference dye is indicative of the quantity of the ROS at the subsequent time point,
e) optionally repeating steps c) to d).

In another aspect, the present disclosure includes a method of measurement of pH comprising
detecting in a subject that had been administered a ratiometric detection composition of the present disclosure:
   a fluorescence level of the reference dye; and
   a fluorescence level of the sensor dye; and
determining a pH value, wherein a value of the fluorescence level of the sensor dye normalized to the fluorescence level of the reference dye is indicative of the pH value.

In another aspect, the present disclosure includes a method for monitoring of pH comprising
a) detecting in a subject that had been administered a ratiometric detection composition of the present disclosure:
   an initial fluorescence level of the reference dye at an initial time point; and
   an initial fluorescence level of the sensor dye at the initial time point;
b) determining a pH value at the initial time point, wherein a value of the initial fluorescence level of the sensor dye normalized to the initial fluorescence level of the reference dye is indicative of the pH value at the initial time point;
c) detecting in the subject:
   a subsequent fluorescence level of the reference dye at a subsequent time point; and
   a subsequent fluorescence level of the sensor dye at the subsequent time point; and
d) determining a pH value at the subsequent time point, wherein a value of the subsequent fluorescence level of the sensor dye normalized to the subsequent fluorescence level of the reference dye is indicative of the pH value at the subsequent time point,
e) optionally repeating steps c) to d).

In another aspect, the present disclosure includes a method of detection of a cation comprising
detecting in a subject that had been administered a ratiometric detection composition of the present disclosure:
   a fluorescence level of the reference dye; and
   a fluorescence level of the sensor dye; and
assessing a presence of the cation, wherein a positive value of the fluorescence level of the sensor dye normalized to the fluorescence level of the reference dye is indicative of the presence of the cation.

In another aspect, the present disclosure includes a method of measurement of a cation comprising
detecting in a subject that had been administered a ratiometric detection composition of the present disclosure:
   a fluorescence level of the reference dye; and
   a fluorescence level of the sensor dye;
determining a quantity of the cation, wherein a value of the fluorescence level of the sensor dye normalized to the fluorescence level of the reference dye is indicative of the quantity of the cation.

In another aspect, the present disclosure includes a method for monitoring of a cation comprising
a) detecting in a subject that had been administered a ratiometric detection composition of the present disclosure:
   an initial fluorescence level of the reference dye at an initial time point; and
   an initial fluorescence level of the sensor dye at the initial time point;
b) determining a quantity of the cation at the initial time point, wherein a value of the initial fluorescence level of the sensor dye normalized to the initial fluorescence level of the reference dye is indicative of the quantity of the cation at the initial time point;
c) detecting in the subject:
   a subsequent fluorescence level of the reference dye at a subsequent time point; and
   a subsequent fluorescence level of the sensor dye at the subsequent time point; and
d) determining a quantity of the cation at the subsequent time point, wherein a value of the subsequent fluorescence level of the sensor dye normalized to the subsequent fluorescence level of the reference dye is indicative of the quantity of the cation at the subsequent time point,
e) optionally repeating steps c) to d) and/or optionally comparing to the quantity of step b).

In another aspect, the present disclosure includes a kit for ratiometric detection/measurement/monitoring of an analyte comprising
a microneedle array of the present disclosure;
an applicator device for applying the microneedle array, optionally a spring-loaded applicator device; and
optionally one or more of instructions for use, and disinfecting wipe.

Other features and advantages of the present disclosure will become apparent from the following detailed description. It should be understood, however, that the detailed description and the specific examples while indicating embodiments of the application are given by way of illustration only, since various changes and modifications within the spirit and scope of the application will become apparent to those skilled in the art from this detailed description.

### DRAWINGS

The disclosure will be described in greater detail herein below with reference to the drawings in which:

FIG. 1 shows Synthesis and characterization of PEGylated Cy5. A) Preparation, reduction, and oxidation of Cy5-PEG; b) Excitation and emission spectra of Cy5, Cy5-PEG and H-Cy5-PEG. Inset shows greatly reduced fluorescence of H-Cy5-PEG; c) Increased water solubility provided by PEGylation of Cy5. Error bars, mean±SD, (n=3), *p≤0.05, calculated using Mann-Whitney non-parametric t-test. Inset shows bright fluorescence of Cy5-PEG compared to low fluorescence and aggregation of free Cy5; d) Stability in aqueous solutions (10 μM) of free Cy5, and Cy5-PEG made from purified and non-purified PEG-NH₂. Error bars, mean±SD, (n=3); e) Sensitivity of free H-Cy5 in solution (10 μM). Error bars, mean±SD, (n=9); f) Sensitivity of H-Cy5-PEG in solution (10 μM). Error bars, mean±SD, (n=9); g) Stability of signal of re-oxidized sensor in aqueous solution (10 μM) over time. Error bars, mean±SD, (n=9), **p≤0.01, calculated using Mann-Whitney non-parametric t-test.

FIG. 2 shows Optimization of MN polymers and evaluation MN manufacture and functionality. A) Stability of free H-Cy5 (10 μM) in common MN polymers. Error bars, mean±SD, (n=6); b) Stability of H-Cy5-PEG (10 μM) in common MN polymers. Error bars, mean±SD, (n=6); c) Force-displacement curve of failure force of MNs with and without presence of H-Cy5-PEG/Cy7.5-PEG. Inset shows MNs before and after deflection. Scale bar=500 μm. Error bars, mean±SD, (n=6); d) Brightfield image of MNs made from ULMW HA/Dextran (8:1); e) SEM of ULMW HA/Dextran MNs. Scale bar=100 μm, or 10 μm for inset; f) Fluorescent image of Cy5-PEG contained within MN tips; g) Microneedle tattoo of Cy5-PEG delivered in vivo to rat skin using MNs; h) Sensitivity of H-Cy5-PEG MNs after re-dissolution (using H₂O₂ as oxidant). Error bars, mean±SD, (n=3); i) Stability of Cy3 and Cy3-PEG (10 μM) in the presence of ROS (Fenton's reagent, FeSO₄/H₂O₂). Error bars, mean±SD, (n=3), ****p<0.0001, calculated using Mann-Whitney non-parametric t-test; j) Sensitivity of H-Cy5-PEG (10 μM) in the presence of Cy3-PEG (2 μM). Error bars mean±SD, (n=3).

FIG. 3 shows a schematic of an illustrative solvent-casting method for fabrication of dissolving polymeric MNs.

FIG. 4 shows reoxidation of H-Cy5 using H₂O₂. When exposed to H₂O₂, the magnitude of the increase in fluorescence of H-Cy5 is greatly reduced, and only occurs at high concentrations (1 mM). Error bars, Mean±SD, (n=3).

FIG. 5 shows Characterization of Cy5-PEG/Cy3-PEG microneedle tattoo delivery to the skin a) Fluorescence intensity of different concentrations of Cy5-PEG delivered to rat skin. Error bars, mean±SD, (n=3); b) Fluorescence intensity of different concentrations of Cy3-PEG delivered to rat skin. Error bars, mean±SD, (n=3); c) Fluorescent image (Cy5 filter) of Cy5-PEG/Cy3-PEG microneedle tattoo (150 μM) delivered to rat skin in vivo. Scale bar=1000 μm; d) Overlaid fluorescent image (Cy3 and Cy5 filters) of Cy5-PEG/Cy3-PEG microneedle tattoo (150 μM) delivered to rat skin in vivo. Scale bar=1000 μm; e) Fluorescent image (Cy3 filter) of Cy5-PEG/Cy3-PEG microneedle tattoo (150 μM) delivered to rat skin in vivo. Scale bar=1000 μm; f) Fluorescent image (Cy5 filter) of Cy5/Cy3 microneedle tattoo (150 μM) delivered to rat skin in vivo. Scale bar=1000 μm; G) Overlaid fluorescent image (Cy3 and Cy5 filters) of Cy5/Cy3 microneedle tattoo (150 μM) delivered to rat skin in vivo. Scale bar=1000 μm; H) Fluorescent image (Cy3 filter) of Cy5/Cy3 microneedle tattoo (150 μM) delivered to rat skin in vivo. Scale bar=1000 μm.

FIG. 6 shows pictures of fluorescent Cy5-PEG MN tattoo. The fluorescent Cy5-PEG MN tattoo is invisible to the naked eye. A) Fluorescence microscopy image (λex=650 nm) of Cy5-PEG MN tattoo (150 μM) in excised porcine skin; b) Brightfield microscopy image of Cy5-PEG MN tattoo in excised porcine skin.

FIG. 7 shows Validation of the inclusion of a fluorescent reference dye: a) Brightfield image of intact MN tips. Scale bar=500 μm; b) Brightfield image of MNs with half of tips removed. Scale bar=500 μm; c) Cy5-PEG fluorescence visible in intact MN tips. Scale bar=500 μm; d) Cy5-PEG fluorescence in MNs with half of tips removed. Scale bar=500 μm; e) Comparison of H-Cy5-PEG fluorescence intensity relative to ratio of H-Cy5-PEG/Cy3-PEG fluorescence, showing no significant difference when whole MN patch is delivered. Error bars, mean±SD, (n=4); f) Comparison of H-Cy5-PEG fluorescence intensity relative to control with ratio of H-Cy5-PEG/Cy3-PEG fluorescence when only half of MN tips delivered to ROS-containing gels. Significant difference observed between H-Cy5-PEG fluorescence and H-Cy5-PEG/Cy3-PEG fluorescence ratio. Error bars, mean±SD, (n=4), **p≤0.01, *p<0.05, calculated using Mann-Whitney non-parametric t-test; g) Comparison of H-Cy5-PEG fluorescence intensity relative to control when delivered using half or whole MN patch. Significant difference observed between half and whole MN patch. Error bars, mean±SD, (n=4), ****p<0.0001, *p≤0.05, calculated using Mann-Whitney non-parametric t-test; h) Comparison of H-Cy5-PEG/Cy3-PEG fluorescence ratio relative to control when delivered using half or whole MN patch. No significant difference observed between half and whole MN patch. Error bars, mean±SD, (n=4).

FIG. 8 shows Linear relationship between relative fluorescence ratio of microneedle tattoo and quantity of ROS in skin-simulating gels. [Fenton's Reagent] refers to the concentration of FeSO₄ used to prepare the Fenton's Reagent.

FIG. 9 shows Re-dissolution of Cy5-PEG MNs before and after application to porcine skin. The reduction in average fluorescence intensity of the MNs after application is less than the variation in fluorescence intensity of individual MNs. Error bars, Mean±SD, (n=4).

FIG. 10 shows Testing microneedle tattoo in human-based atopic model of skin inflammation: a) Addition of IL-4 and IL-13 to FLG-skin cells emulates inflammatory conditions; b) Histological image of inflamed FLG-skin model; c) IVIS image of microneedle tattoo in normal (control) and inflammatory filaggrin-deficient (FLG−/−) skin models using Cy5 and Cy7.5 filter; d) Fluorescence intensity of H-Cy5-PEG microneedle tattoo in normal and inflammatory filaggrin-deficient (FLG-) skin models. Error bars, mean±SD, (n=4); e) Fluorescence ratio of H-Cy5-PEG/Cy7.5-PEG microneedle tattoo in normal and inflammatory filaggrin-deficient (FLG-) skin model. Error bars, mean±SD, (n=4), **p≤0.01, calculated using two-way ANOVA with Tukey's post-hoc HSD correction.

FIG. 11 shows Stability of Cy7.5-PEG ex vivo and in vivo: a) In a human-based atopic model of skin inflammation, Cy7.5-PEG fluorescence intensity did not decrease over the course of time studied (20 minutes); b) In UV-irradiated rats and non-irradiated control rats, variation in Cy7.5-PEG fluorescence is not correlated with the presence of ROS.

FIG. 12 shows the Validation of portable fluorescence reader and in vivo testing: a) Image of portable fluorescence reader; b) Linear dynamic range of portable fluorescence reader when measuring solutions of Cy5 or Cy7.5. Error bars, mean±SD, (n=3); c) Linear dynamic range of portable fluorescence reader when measuring Cy5-PEG or Cy7.5-PEG microneedle tattoo in excised porcine skin. Error bars, mean±SD, (n=4); d) Fluorescence intensity (at Cy5 and Cy7.5 wavelengths) of H-Cy5-PEG/Cy7.5-PEG microneedle tattoo in excised porcine skin, measured suing portable fluorescence reader. Error bars, mean±SD, (n=9); e) Diagram showing process of in vivo testing: rats were exposed to UVB light, H-Cy5-PEG/Cy7.5-PEG microneedle tattoo was applied, fluorescence was measured using portable fluorescence reader; f) Fluorescence ratio of H-Cy5-PEG/Cy7.5-PEG microneedle tattoo in UVB-irradiated and non-irradiated rats. Error bars, mean±SD, (n=3), ****p<0.0001, **p≤0.01, calculated using two-way ANOVA with Tukey's post-hoc HSD correction.

FIG. 13 shows an exemplary embodiment of a portable multi-wavelength fluorescence reader. A) Cross-section of portable fluorescence reader showing components (exchangeable module is highlighted). 1) pinhole (diameter=4 mm), 2) aluminum heat sink and mounting bracket for transistor outline—56 laser diodes, 3) photodiode printed circuit board, 4) main connector from housing to laser diode and filter module, 5) nitrile butadiene rubber o-ring, 6) laser diode driver with temperature sensor mounted on heat sink, 7) band-pass filter mount (suitable for filters with a diameter of 12.7 mm installed in a housing), 8) lens brackets screwed directly to the housing, 9) fresnel lens, 10) square plano concave lens for cylindrical beam divergence correction and biconcave lens for laser beam diffusion; b) Image of portable fluorescence reader; c) Image of display output of portable fluorescence reader.

FIG. 14 shows A) Structure of novel PET-based fluorescent pH sensor; B) Fluorescence ratio of MTP-Cy7-COOH (sensor)/Cy5 as a function of pH in skin-simulating agarose gels.

FIG. 15 shows A) Structure of novel PET-based fluorescent Na⁺ sensor; B) Fluorescence ratio of Benzo-15-crown-5-Cy7-COOH (sensor)/Cy5 as a function of pH in skin-simulating agarose gels.

FIG. 16 shows bar graphs of fluorescence intensity of reoxidation of H-Cy5-TMC to Cy5-TMC in H₂O with FeSO₄ and H₂O₂ at concentrations between 10 μM to 1 mM.

FIG. 17 shows bar graphs of fluorescence intensity of reoxidation of H-Cy5-PEI to Cy5-PEI in H₂O with FeSO₄ and H₂O₂ at concentrations between 10 μM to 1 mM.

FIG. 18 shows an illustrative embodiment of microneedle administered topically to a subject's wrist and the monitoring of analytes through the sensor dyes in the microneedle using a portable and/or wearable device. The output can be for example transferred to a second device such as a mobile phone.

FIG. 19 shows an illustrative embodiment of microneedles containing either a sensor dye, or a PEGylated sensor dye and PEGylated reference dye, allowing for ratiometric monitoring

FIG. 20 shows the structure of Cy5-PEG, (a) its redox reaction, and (b) the change in fluorescence Amax in the oxidized vs reduced state.

FIG. 21 shows a graph showing correlation between pH value (x-axis) and the relative fluorescence ratio (y-axis) using Pip-Cy7-PEG as the sensor dye and Cy7.5-PEG as reference dye.

### DESCRIPTION OF VARIOUS EMBODIMENTS

### 1. Definitions

Unless otherwise indicated, the definitions and embodiments described in this and other sections are intended to be applicable to all embodiments and aspects of the application herein described for which they are suitable as would be understood by a person skilled in the art. Unless otherwise specified within this application or unless a person skilled in the art would understand otherwise, the nomenclature used in this application generally follows the examples and rules stated in “Nomenclature of Organic Chemistry” (Pergamon Press, 1979), Sections A, B, C, D, E, F, and H. Optionally, a name of a compound may be generated using a chemical naming program: ACD/ChemSketch, Version 5.09/September 2001, Advanced Chemistry Development, Inc., Toronto, Canada.

As used in the present disclosure, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The term “suitable” as used herein means that the selection of the particular compound or conditions would depend on the specific synthetic manipulation to be performed, and the identity of the species to be transformed, but the selection would be well within the skill of a person trained in the art. All method steps described herein are to be conducted under conditions sufficient to provide the desired product. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

In embodiments of the present disclosure, the compounds described herein may have at least one asymmetric center. Where compounds possess more than one asymmetric center, they may exist as diastereomers. It is to be understood that all such isomers and mixtures thereof in any proportion are encompassed within the scope of the present disclosure. It is to be further understood that while the stereochemistry of the compounds may be as shown in any given compound listed herein, such compounds may also contain certain amounts (for example, less than 20%, suitably less than 10%, more suitably less than 5%) of compounds of the present disclosure having alternate stereochemistry. It is intended that any optical isomers, as separated, pure or partially purified optical isomers or racemic mixtures thereof are included within the scope of the present disclosure.

The compounds of the present disclosure may also exist in different tautomeric forms and it is intended that any tautomeric forms which the compounds form are included within the scope of the present application.

The compounds of the present disclosure may further exist in varying polymorphic forms and it is contemplated that any polymorphs which form are included within the scope of the present application.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The term “dye of the present disclosure” or “dyes of the present disclosure” or the like as used herein refers to a sensor dye or a reference dye of the present disclosure as described herein.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₆alkyl means an alkyl group having 1, 2, 3, 4, 5 or 6 carbon atoms. All alkyl groups are optionally fluorosubstituted unless otherwise stated.

The term “alkenyl” as used herein, whether it is used alone or as part of another group, means straight or branched chain, unsaturated alkyl groups containing at least one double bond. The number of carbon atoms that are possible in the referenced alkyl group are indicated by the prefix “Cₙ₁₋ₙ₂”. For example, the term C₂₋₆alkenyl means an alkenyl group having 2, 3, 4, 5 or 6 carbon atoms.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms.

The term “alkylene” as used herein, whether it is used alone or as part of another group, means a straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₁₋₆alkylene means an alkylene group having 1, 2, 3, 4, 5 or 6 carbon atoms. All alkylene groups are optionally fluorosubstituted unless otherwise stated.

The term “cycloalkyl,” as used herein, whether it is used alone or as part of another group, means a saturated carbocyclic group containing one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “aryl” as used herein, whether it is used alone or as part of another group, refers to carbocyclic groups containing at least one aromatic ring. In an embodiment of the application, the aryl group contains from 6, 9 or 10 carbon atoms, such as phenyl, indanyl or naphthyl.

The term “heterocycloalkyl” as used herein, whether it is used alone or as part of another group, refers to cyclic groups containing at least one non-aromatic ring in which one or more of the atoms are a heteroatom selected from O, S and N. Heterocycloalkyl groups are either saturated or unsaturated (i.e. contain one or more double bonds). When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above.

The term “heteroaryl” as used herein, whether it is used alone or as part of another group, refers to cyclic groups containing at least one heteroaromatic ring in which one or more of the atoms are a heteroatom selected from O, S and N. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above.

All cyclic groups, including aryl and cyclo groups, contain one or more than one ring (i.e. are polycyclic). When a cyclic group contains more than one ring, the rings may be fused, bridged, spirofused or linked by a bond.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “halo” as used herein refers to a halogen atom and includes fluoro, chloro, bromo and iodo.

The term “optionally substituted” refers to groups, structures, or molecules that are either unsubstituted or are substituted with one or more substituents.

The term “fluorosubstituted” refers to the substitution of one or more, including all, hydrogens in a referenced group with fluorine.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3^{rd} Edition, 2003, Georg Thieme Verlag (The Americas).

The symbols “ [symbol] ”, “ [symbol] ” or the like as used herein refer to optionally present bonds and atoms.

The term “subject” as used herein includes all members of the animal kingdom including mammals, and suitably refers to humans. Thus the methods and uses of the present disclosure are applicable to both human therapy and veterinary applications.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. Either the mono- or di-acid salts can be formed, and such salts can exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, EGFRaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. [See, for example, S. M. Berge, et al., “Pharmaceutical Salts,” J. Pharm. Sci. 1977, 66, 1-19]. The selection of the appropriate salt may be useful so that an ester functionality, if any, elsewhere in a compound is not hydrolyzed. The selection criteria for the appropriate salt will be known to one skilled in the art.

The term “inert organic solvent” as used herein refers to a solvent that is generally considered as non-reactive with the functional groups that are present in the compounds to be combined together in any given reaction so that it does not interfere with or inhibit the desired synthetic transformation. Organic solvents are typically non-polar and dissolve compounds that are non soluble in aqueous solutions.

The term “MNs” or “polymeric MNs” or “polymeric microneedles” as used herein refers to microneedles of the present disclosure.

II. Dyes and Microneedles of the Disclosure

In an aspect, the present disclosure includes a ratiometric detection composition comprising
a reference dye, the reference dye being a first cyanine dye that is PEGylated; and
a sensor dye, the sensor dye being a second cyanine dye that is PEGylated and that comprises an analyte sensing moiety;
wherein the reference dye is inert towards the analyte;
wherein an emission maximum wavelength of the reference dye and an emission maximum wavelength of the sensor dye are at least about 50 nm apart;
wherein the first cyanine dye and the second cyanine dye are PEGylated with PEG groups of substantially the same molecular weight.

In some embodiments, the emission maximum wavelength of the reference dye and the emission maximum wavelength of the sensor dye are at least about 60 nm, at least 70 nm, at least 80 nm, at least 90 nm, at least 100 nm, at least 150 nm, at least 200 nm apart. In some embodiments, the emission maximum wavelength of the reference dye and the emission maximum wavelength of the sensor dye are less than 300 nm, less than 250 nm, less than 200 nm, less than 150 nm, less than 100 nm, less than 90 nm, less than 80 nm, less than 70 nm apart.

In some embodiments, the reference dye has a structure of Formula I

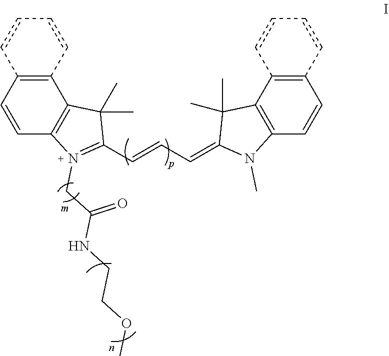

or a salt thereof,
wherein p is an integer from 1 to 3, m is an integer from 2 to 8, n is an integer from 50 to 900, and wherein the

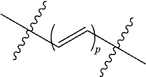

of the compound is optionally substituted with C₁₋₆alkyl or C₁₋₆cycloalkyl.

In some embodiments, the reference dye of Formula I is

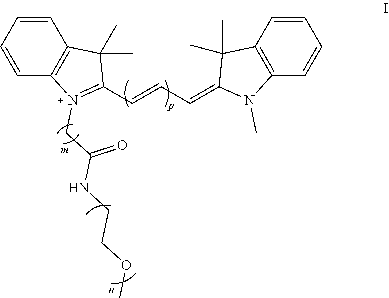

or a salt thereof,
wherein p is an integer from 1 to 3, m is an integer from 2 to 8, n is an integer from 50 to 900, and wherein the

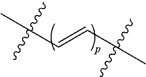

of the compound is optionally substituted with C₁₋₆alkyl or C₁₋₆cycloalkyl.

In some embodiments, the analyte sensing moiety is a reactive oxygen species (ROS) sensing moiety, and the sensor dye has a structure of Formula II

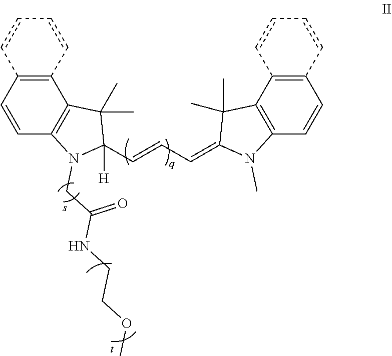

or a salt thereof,
wherein q is an integer from 1 to 3, s is an integer from 2 to 8, t is an integer from 50 to 900, and wherein the

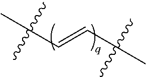

of the compound is optionally substituted with C₁₋₆alkyl or C₁₋₆cycloalkyl.

In some embodiments, the sensor dye of Formula II is

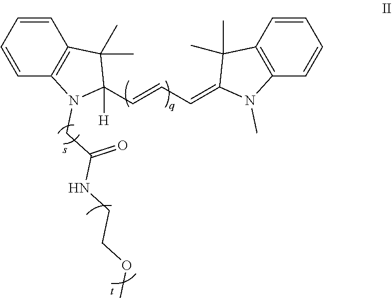

or a salt thereof,
wherein q is an integer from 1 to 3, s is an integer from 2 to 8, t is an integer from 50 to 900, and wherein the

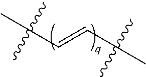

of the compound is optionally substituted with C₁₋₆alkyl or C₁₋₆cycloalkyl.

In some embodiments, p is 1, and/or m is 4 to 6, optionally 5. In some embodiments. q is 2, and/or s is 4 to 6, optionally 5.

In some embodiments, n and t are each independently about 50 to about 800, about 50 to about 700, about 50 to about 650, about 50 to about 600, about 50 to about 550, about 50 to about 500, about 50 to about 450, about 50 to about 400, about 50 to about 350, about 50 to about 300, about 80 to about 250, about 80 to about 200, about 90 to about 150, about 90 to about 130, or about 110. In some embodiments, n and t are each independently at least 50, at least 60, at least 70, at least 80, at least 90, or at least 100. In some embodiments, n and t are each independently less than 700, less than 650, less than 600, less than 550, less than 500, less than 450, less than 400, less than 350, less than 300, less than 250, less than 200, less than 150, or less than 130.

In some embodiments, the sensor dye has a structure of Formula IIIa or IIIb

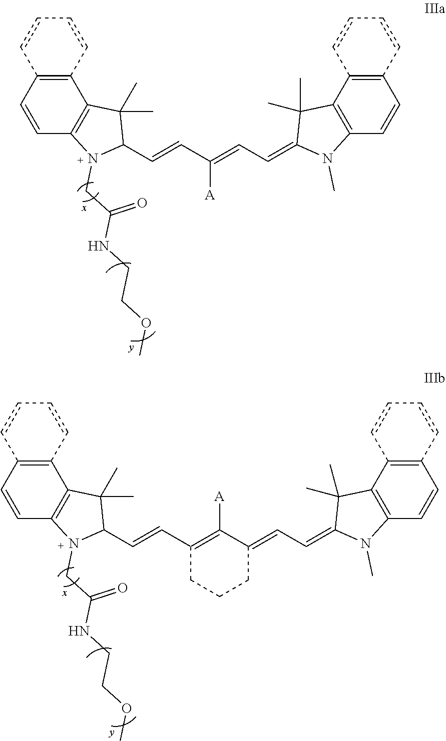

or a salt thereof,
wherein each x is independently an integer from 2 to 8, y is an integer from 50 to 900,
wherein each A is independently the analyte sensing moiety.

In some embodiments, the sensor dye of Formula IIIa is

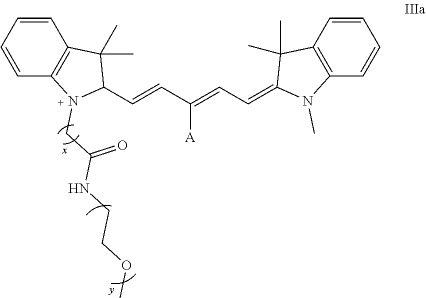

or a salt thereof,
wherein each x is independently an integer from 2 to 8, y is an integer from 50 to 900;
wherein each A is independently the analyte sensing moiety.

In some embodiments, the sensor dye of Formula IIIb is

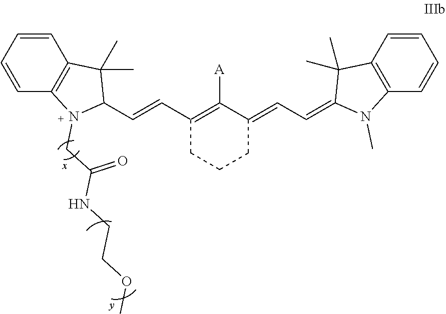

or a salt thereof,
wherein each x is independently an integer from 2 to 8, y is an integer from 50 to 900;
wherein each A is independently the analyte sensing moiety.

In some embodiments, the analyte sensing moiety is selected from pH sensing moiety or cation sensing moiety.

In some embodiments, the pH sensing moiety comprises a terpyridine and/or a piperazine.

In some embodiments, the sensor dye has a structure of Formula IIIb1

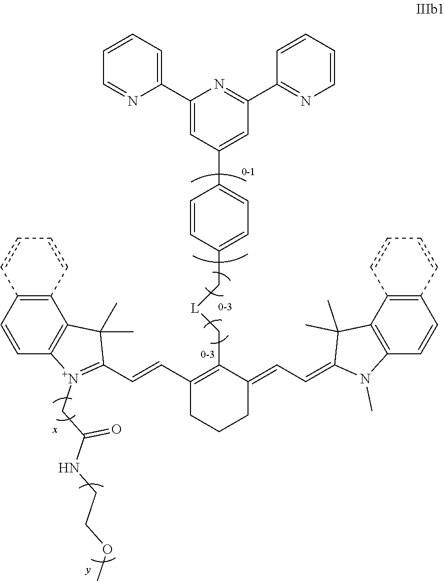

or a salt thereof,
wherein x is an integer from 2 to 8, y is an integer from 50 to 900, L is O, NH, or N(C₁₋₃alkyl).

In some embodiments, the sensor dye of Formula IIIb1 is

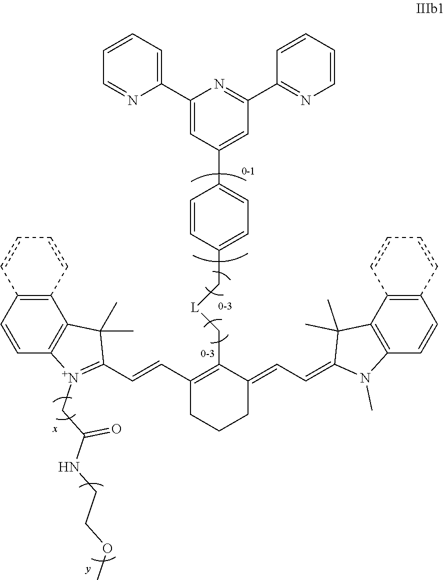

or a salt thereof,
wherein x is an integer from 2 to 8, y is an integer from 50 to 900, L is O, NH, or N(C₁₋₃alkyl).

In some embodiments. the cation sensing moiety comprises a crown ether or an aza crown ether. In some embodiments, the crown ether is selected from 12-crown-4, 15-crown-5, and 18-crown-6. In some embodiments, the aza crown ether is aza-15-crown 5.

In some embodiments, the cation sensing moiety is Li⁺ sensing and the crown ether is 12-crown-4.

In some embodiments, the sensor dye has a structure of Formula IIIb2

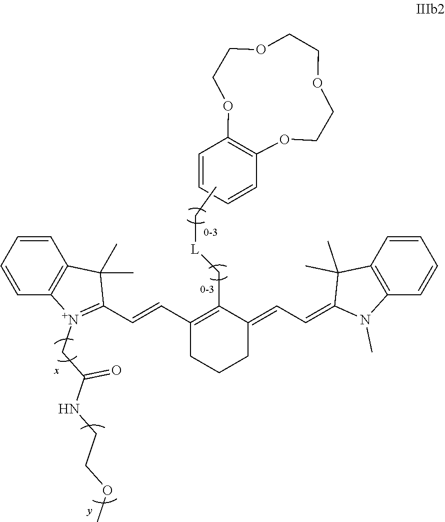

or a salt thereof,
wherein x is an integer from 2 to 8, y is an integer from 50 to 900, and L is O, NH, N(C₁₋₃alkyl), or phenyl.

In some embodiments, the sensor dye of Formula IIIb2 is

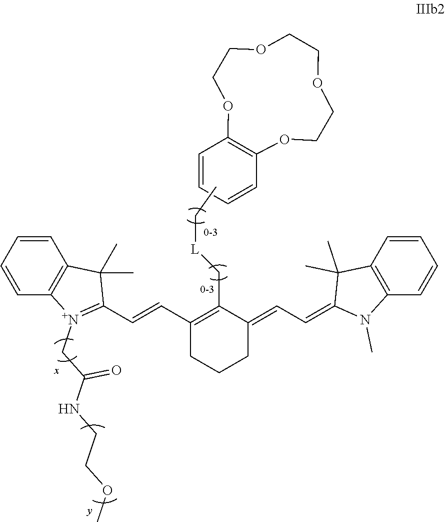

or a salt thereof,
wherein x is an integer from 2 to 8, y is an integer from 50 to 900, and L is O, NH, N(C₁₋₃alkyl), or phenyl.

In some embodiments, the cation sensing moiety is Na⁺ sensing and the crown ether is 15-crown-5. In some embodiments, the cation sensing moiety is Na⁺ sensing and the aza crown ether is aza-15-crown-5.

In some embodiments, the sensor dye has a structure of Formula IIIb3

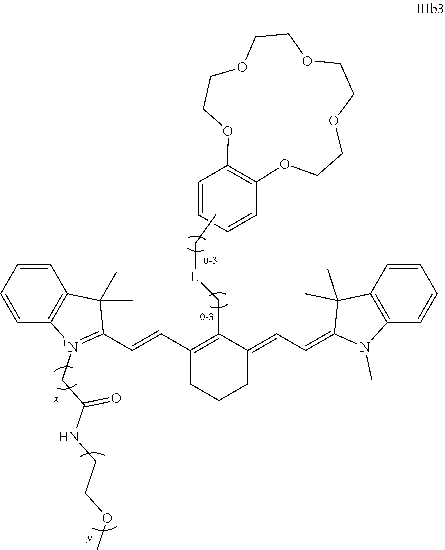

or a salt thereof,
wherein x is an integer from 2 to 8, y is an integer from 50 to 900, and L is O, NH, N(C₁₋₃alkyl), or phenyl.

In some embodiments, the sensor dye of Formula IIIb3

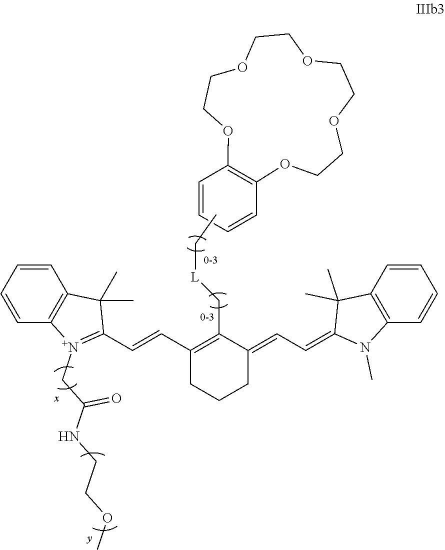

or a salt thereof,
wherein x is an integer from 2 to 8, y is an integer from 50 to 900, and L is O, NH, N(C₁₋₃alkyl), or phenyl.

In some embodiments, cation sensing moiety is K⁺ sensing and the crown ether is 18-crown-6.

In some embodiments, the sensor dye has a structure of Formula IIIb4

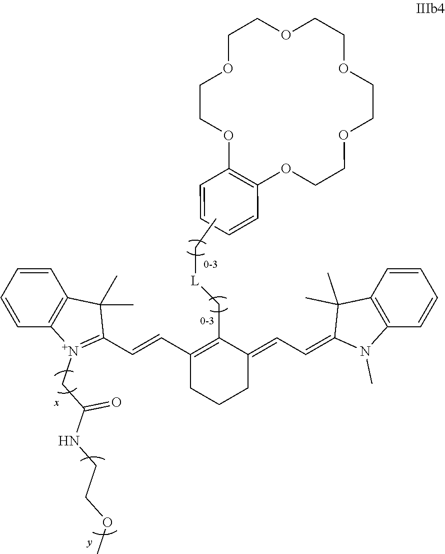

or a salt thereof,
wherein x is an integer from 2 to 8, y is an integer from 50 to 900, and L is O, NH, N(C₁₋₃alkyl), or phenyl.

In some embodiments, the sensor dye of Formula IIIb4 is

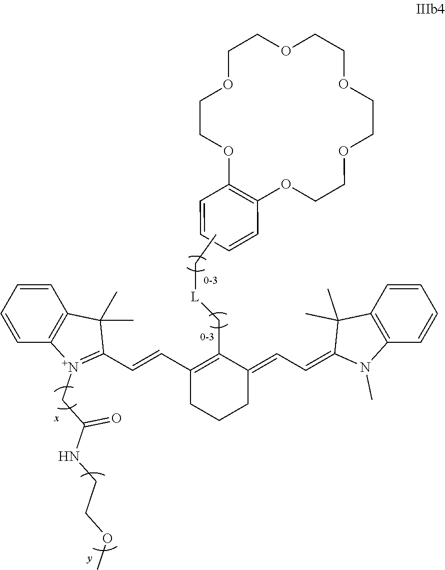

or a salt thereof,
wherein x is an integer from 2 to 8, y is an integer from 50 to 900, and L is O, NH, N(C₁₋₃alkyl), or phenyl.

In some embodiments, the sensor dye has a structure of Formula IIIb5

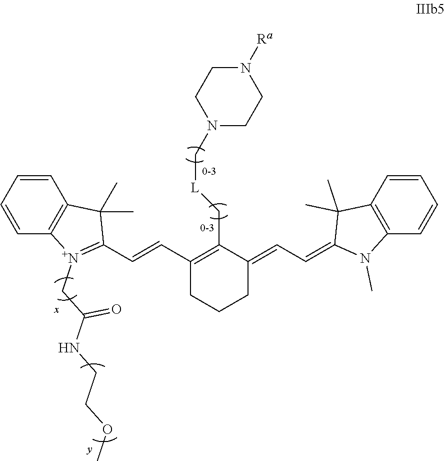

or a salt thereof,
wherein x is an integer from 2 to 8, y is an integer from 50 to 900, L is absent, O, NH, N(C₁₋₃alkyl), or aryl, or heteroaryl, and R^{a} is C₁₋₆ alkyl.

In some embodiments, R^{a} is C₁₋₆ alkyl. In some embodiments, R^{a} is methyl.

In some embodiments, the heteroaryl in L is triazole. In some embodiments, L is absent. In some embodiments, the sensor dye has a structure of

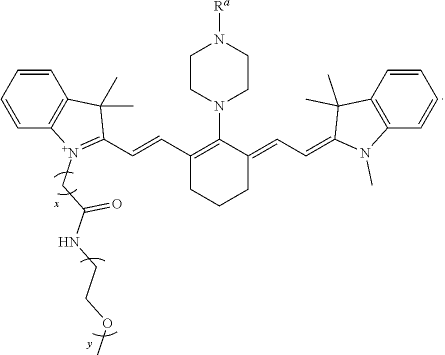

In some embodiments, L is absent and R^{a} is methyl. In some embodiments, the sensor dye has a structure of

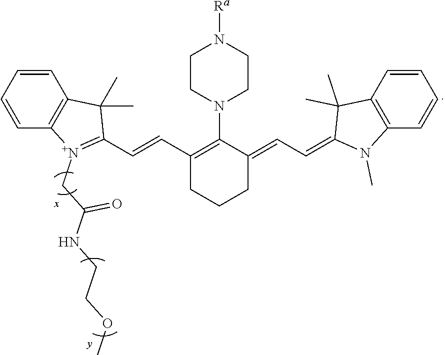

In some embodiments, the sensor dye has a structure of Formula IIIb6

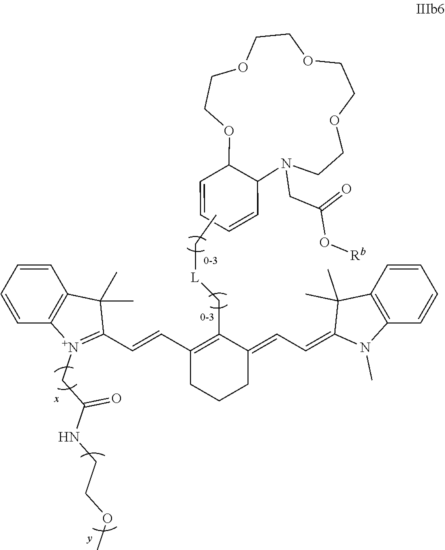

or a salt thereof
wherein x is an integer from 2 to 8, y is an integer from 50 to 900, L is O, NH, N(C₁₋₃alkyl), phenyl, or heteroaryl, and R^{b} is C₁₋₆ alkyl.

In some embodiments, the heteroaryl of L is triazole.

In some embodiments, the sensor dye has a structure of

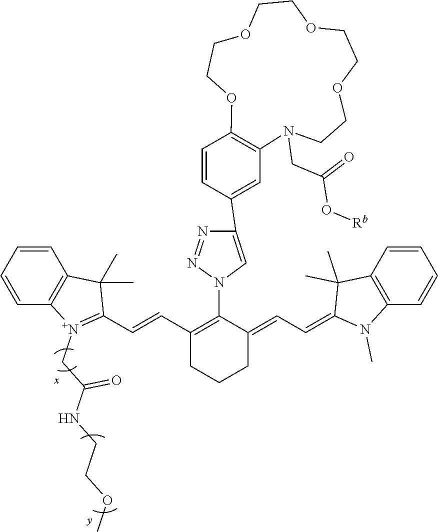

or a salt thereof.

In some embodiments, R^{b} is a C₁₋₃ alkyl. In some embodiments, R^{b} is methyl.

In some embodiments, x is 4 to 6, optionally 5.

In some embodiments, y is about 50 to about 800, about 50 to about 700, about 50 to about 650, about 50 to about 600, about 50 to about 550, about 50 to about 500, about 50 to about 450, about 50 to about 400, about 50 to about 350, about 50 to about 300, about 80 to about 250, about 80 to about 200, about 90 to about 150, about 90 to about 130, or about 110.

In some embodiments, p is 2, m is 4 to 6, optionally 5, and/or n is about 50 to about 800, about 50 to about 700, about 50 to about 650, about 50 to about 600, about 50 to about 550, about 50 to about 500, about 50 to about 450, about 50 to about 400, about 50 to about 350, about 50 to about 300, about 80 to about 250, about 80 to about 200, about 90 to about 150, about 90 to about 130, or about 110.

In some embodiments, the reference dye is

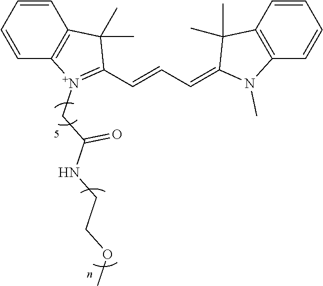

or a salt thereof,
wherein n is about 90 to about 130, optionally about 110.

In some embodiments, the sensor dye is

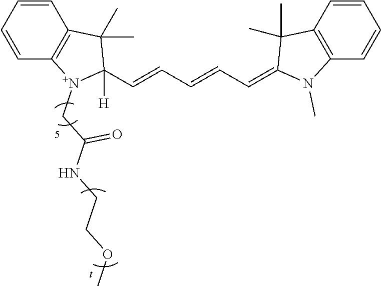

or a salt thereof,
wherein t is about 90 to about 130, optionally about 110.

In some embodiments, the reference dye is

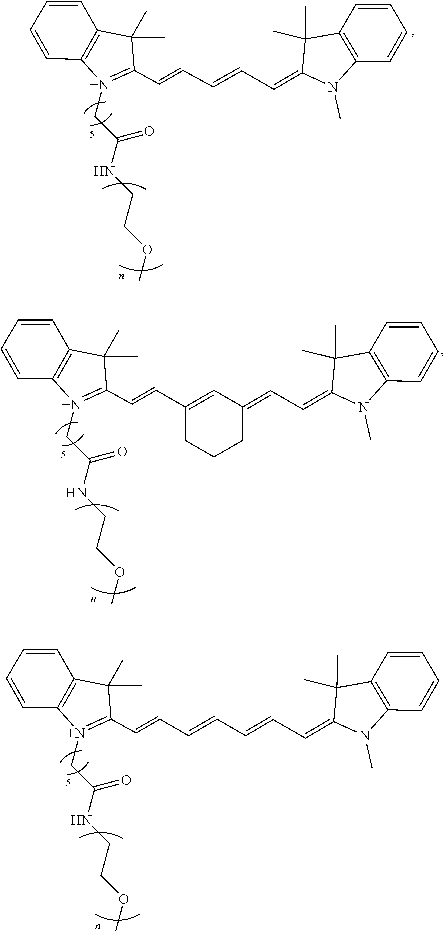

or a salt thereof,
wherein n is about 90 to about 130, optionally about 110.

In some embodiments, the sensor dye is selected from

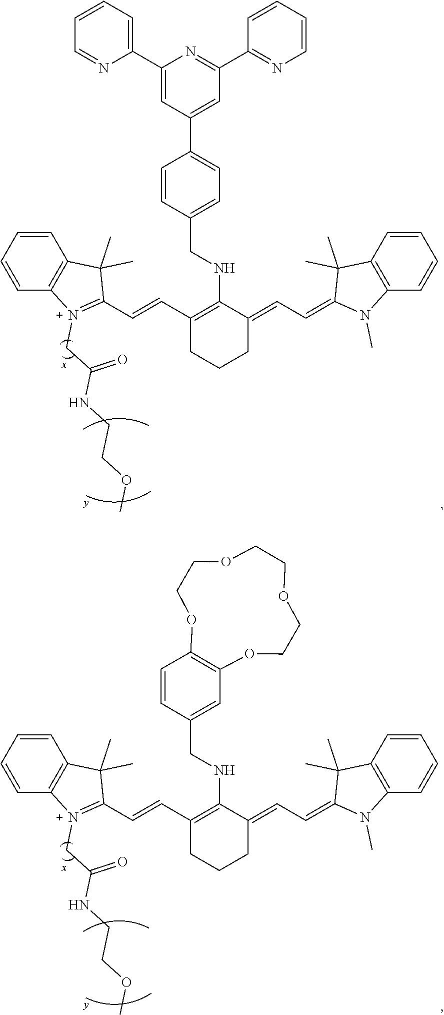

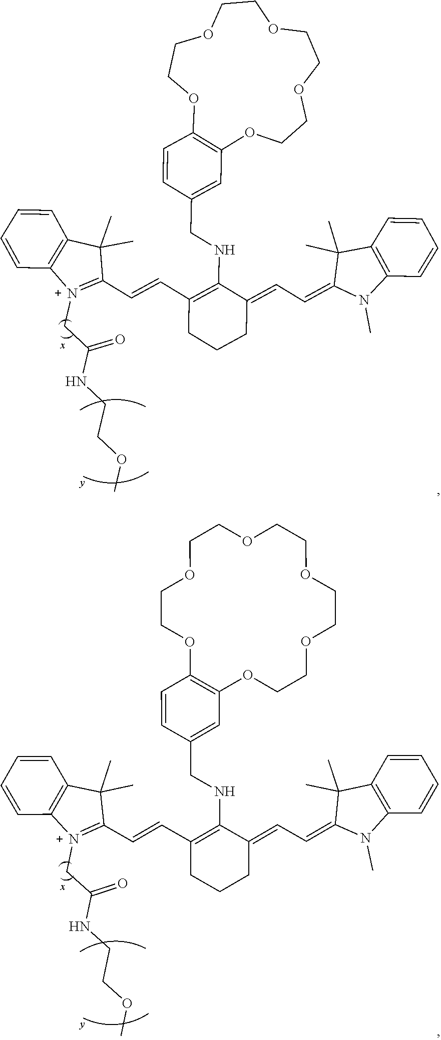

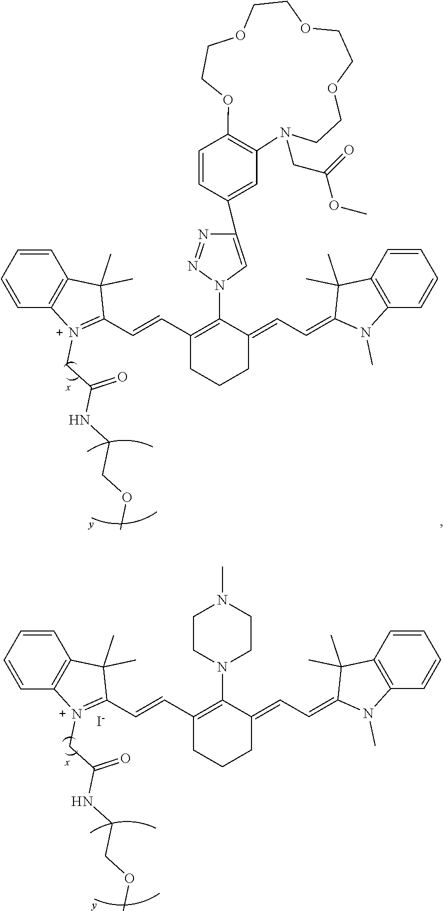

or salt thereof,
wherein x is 5 and y is about 90 to about 130, optionally about 110.

In some embodiments, the ratiometric detection composition of any the present disclosure comprising a plurality of sensor dyes of the present disclosure.

In another aspect, the present disclosure includes a reference dye compound as defined in the present disclosure.

In another aspect, the present disclosure includes a sensor dye compound as defined in the present disclosure.

In another aspect, the present disclosure includes a microneedle array comprising
a backing layer, and
a plurality of microneedles each having a first end and a second sharpened end for penetration into skin, the plurality of microneedles extending outwardly from the backing layer at the first end;
wherein the microneedles are formed with a biodegradable polymer and comprise a ratiometric detection composition of the present disclosure that is incorporated in the biodegradable polymer;
wherein the plurality of microneedles is dissolvable and releasable from the backing layer after penetration into the skin.

The microneedles can be pyramidal projections as described in the Examples or other shapes including for example cone shaped projections and the like. The first end that contacts the backing layer typically has larger dimensions than the second sharpened end. As described herein the microneedles having a first end and second sharpened end can for example be prepared by mold casting.

In some embodiments, the biodegradable polymer is selected from polyvinylpyrrolidinone (PVP), polyvinylalcohol (PVA), low molecular weight hyaluronic acid, optionally ultra-low molecular weight hyaluronic acid, and super-low molecular weight hyaluronic acid, silk fibroin, maltose, poly(vinyl methyl ether)-co-maleic anhydride (PVME/MA), chitin, carboxymethylcellulose and its sodium salt, chondroitin sulfate and its sodium salt, sodium alginate, hydroxypropylcellulose, hydroxypropylmethylcellulose, dextran, and combinations thereof.

In some embodiments, the microneedles each comprises about 50 μM to about 500 μM, about 75 μM to about 400 μM, about 100 μM to about 300 μM, or about 150 μM of the sensor dye, and about 50 μM to about 300 μM, about 75 μM to about 250 μM, about 80 μM to about 200 μM, or about 100 μM of the reference dye.

In some embodiments, the microneedles each comprises the sensor dye and reference dye at a ratio of about 3 to about 2.

It can be appreciated that the concentrations and ratios can be adjusted based on for example the nature of the analyte, and properties of the sensor dye and reference dye (such as brightness, sensitivity, etc.).

In some embodiments, the backing layer is or comprises a biodegradable polymer selected from polyvinylpyrrolidinone (PVP), polyvinylalcohol (PVA), low molecular weight hyaluronic acid, optionally ultra-low molecular weight hyaluronic acid, and super-low molecular weight hyaluronic acid, silk fibroin, maltose, poly(vinyl methyl ether)-co-maleic anhydride (PVME/MA), chitin, carboxymethylcellulose and its sodium salt, chondroitin sulfate and its sodium salt, sodium alginate, hydroxypropylcellulose, hydroxypropylmethylcellulose, dextran, and combinations thereof.

In some embodiments, the microneedles are of a length sufficient to penetrate a stratum corneum layer and to reach an epidermis layer, optionally a dermis layer, optionally wherein the microneedles have a length of from about 200 nm to about 1500 nm, about 250 nm to about 1200 nm, or about 300 nm to about 1000 nm. In some embodiments, the microneedles have a length of at least 100 nm, at least 200 nm, at least 400 nm. In some embodiments, the microneedles have a length of less than 2000 nm, less than 1800 nm, less than 1500 nm, less than 1000 nm.

The dyes of the present disclosure are suitably formulated in a conventional manner into compositions using one or more carriers. Accordingly, the present disclosure also includes a composition comprising one or more dyes of the application and a carrier. The dyes of the disclosure are suitably formulated into pharmaceutical compositions for administration to subjects in a biologically compatible form suitable for administration in vivo. Accordingly, the present disclosure further includes a pharmaceutical composition comprising one or more compounds of the application and a pharmaceutically acceptable carrier.

The dyes of the disclosure may be administered to a subject in a variety of forms depending on the selected route of administration, as will be understood by those skilled in the art. A dye of the disclosure may be administered, for example, by patch, intradermal, dermal or transdermal administration and the pharmaceutical compositions formulated accordingly. Administration can be by means of a pump for periodic or continuous delivery. The dyes of the disclosure may be administered topically, for example by a patch comprising the microneedle array of the present disclosure. It is contemplated that administration of the dyes and/or the ratiometric detection composition comprises delivering the dyes through the stratum corneum to for example the dermal interstitial fluid in the epidermis and/or the dermis layer of the skin. For example, it is contemplated that the microneedles pierce the top surface of the skin (e.g. the stratum corneum). Thus, it is contemplated that the microneedles of the resent disclosure are of sufficient length to penetrate the top layer of the skin (e.g. the stratum corneum) and/or to reach the epidermis and/or the dermis.

The dyes of the disclosure including pharmaceutically acceptable salts, solvates and prodrugs thereof are suitably used on their own but will generally be administered in the form of a pharmaceutical composition in which the one or more dyes of the disclosure is in association with a pharmaceutically acceptable carrier. Depending on the mode of administration, the pharmaceutical composition will comprise from about 0.05 wt % to about 99 wt % or about 0.10 wt % to about 70 wt %, of the dye (one or more dyes of the disclosure), and from about 1 wt % to about 99.95 wt % or about 30 wt % to about 99.90 wt % of a pharmaceutically acceptable carrier, all percentages by weight being based on the total composition.

Dyes of the disclosure may be used alone or in combination with other known agents useful for detection, measurement and/or monitoring of analytes. When used in combination with other agents, it is an embodiment that the dyes of the disclosure are administered contemporaneously with those agents. As used herein, “contemporaneous administration” of two substances to a subject means providing each of the two substances so that they are both biologically active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present disclosure that a combination of agents is administered to a subject in a non-contemporaneous fashion. In an embodiment, a dye of the present disclosure is administered with another therapeutic agent simultaneously or sequentially in separate unit dosage forms or together in a single unit dosage form. Accordingly, the present disclosure provides a single unit dosage form comprising one or more dyes of the application, an additional therapeutic agent, and a pharmaceutically acceptable carrier.

The dosage of dyes of the disclosure can vary depending on many factors such as the pharmacodynamic properties of the dyes, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. As a representative example, dosages of one or more dyes of the disclosure will range between about 1 mg per administration to about 1000 mg per administration for an adult, suitably about 1 mg per administration to about 500 mg per administration, more suitably about 1 mg per day to about 200 mg per administration.

To be clear, in the above, the term “a dye” also includes embodiments wherein one or more dyes are referenced.

III. Methods, Uses and Kits of the Disclosure

In another aspect, the present disclosure includes the ratiometric detection composition, a reference dye and/or a sensor dye of the present disclosure for use in a ratiometric detection, measurement and/or monitoring of an analyte.

In another aspect, the present disclosure includes the ratiometric detection composition, a reference dye and/or a sensor dye of the present disclosure for use in a ratiometric detection, measurement and/or monitoring of a plurality of analytes.

In another aspect, the present disclosure includes a method of delivering a ratiometric detection composition to a subject comprising administering a microneedle array of the present disclosure to a skin of the subject.

In some embodiments, the method further comprising removing the backing layer from the skin after a desired period of time, optionally after approximately 1 to 10 minutes.

In another aspect, the present disclosure includes a method of detection of an analyte comprising
detecting in a subject that had been administered a ratiometric detection composition of the present disclosure:
   a fluorescence level of the reference dye;
   a fluorescence level of the sensor dye;
assessing a presence of the analyte, wherein a positive value of the fluorescence level of the sensor dye normalized to the fluorescence level of the reference dye is indicative of the presence of the analyte.

In some embodiments, the method is for ratiometric detection of a plurality of analytes, and the ratiometric detection composition comprises a plurality of sensor dyes.

In another aspect, the present disclosure includes a method of measurement of an analyte comprising
detecting in a subject that had been administered a ratiometric detection composition of the present disclosure:
   a fluorescence level of the reference dye; and
   a fluorescence level of the sensor dye; and
determining a quantity of the analyte, wherein a value of the fluorescence level of the sensor dye normalized to the fluorescence level of the reference dye is indicative of the quantity of the analyte.

In another aspect, the present disclosure includes a method for monitoring of an analyte comprising
a) detecting in a subject that had been administered a ratiometric detection composition of the present disclosure:
   an initial fluorescence level of the reference dye at an initial time point; and
   an initial fluorescence level of the sensor dye at the initial time point;
b) determining a quantity of the analyte at the initial time point, wherein a value of the initial fluorescence level of the sensor dye normalized to the initial fluorescence level of the reference dye is indicative of the quantity of the analyte at the initial time point;
c) detecting:
   a subsequent fluorescence level of the reference dye at a subsequent time point; and
   a subsequent fluorescence level of the sensor dye at the subsequent time point;
d) determining a quantity of the analyte at the subsequent time point, wherein a value of the subsequent fluorescence level of the sensor dye normalized to the subsequent fluorescence level of the reference dye is indicative of the quantity of the analyte at the subsequent time point; and
e) optionally repeating steps c) to d).

In another aspect, the present disclosure includes a method of detection of ROS comprising
detecting in a subject that had been administered a ratiometric detection composition of the present disclosure:
   a fluorescence level of the reference dye; and
   a fluorescence level of the sensor dye; and
assessing a presence of the ROS, wherein a positive value of the fluorescence level of the sensor dye normalized to the fluorescence level of the reference dye is indicative of the presence of the ROS.

In another aspect, the present disclosure includes a method of measurement of ROS comprising
detecting in a subject that had been administered a ratiometric detection composition of the present disclosure:
   a fluorescence level of the reference dye; and
   a fluorescence level of the sensor dye; and
determining a quantity of the ROS, wherein a value of the fluorescence level of the sensor dye normalized to the fluorescence level of the reference dye is indicative of the quantity of the ROS.

In another aspect, the present disclosure includes a method for monitoring of ROS comprising
a) detecting in a subject that had been administered a ratiometric detection composition of the present disclosure:
   an initial fluorescence level of the reference dye at an initial time point; and
   an initial fluorescence level of the sensor dye at the initial time point;
b) determining a quantity of the analyte at the initial time point, wherein a value of the initial fluorescence level of the sensor dye normalized to the initial fluorescence level of the reference dye is indicative of the quantity of the ROS at the initial time point;
c) detecting in the subject:
   a subsequent fluorescence level of the reference dye at a subsequent time point; and
   a subsequent fluorescence level of the sensor dye at the subsequent time point; and
d) determining a quantity of the analyte at the subsequent time point, wherein a value of the subsequent fluorescence level of the sensor dye normalized to the subsequent fluorescence level of the reference dye is indicative of the quantity of the ROS at the subsequent time point,
e) optionally repeating steps c) to d).

In another aspect, the present disclosure includes a method of measurement of pH comprising
detecting in a subject that had been administered a ratiometric detection composition of the present disclosure:
   a fluorescence level of the reference dye; and
   a fluorescence level of the sensor dye; and
determining a pH value, wherein a value of the fluorescence level of the sensor dye normalized to the fluorescence level of the reference dye is indicative of the pH value.

In another aspect, the present disclosure includes a method for monitoring of pH comprising
a) detecting in a subject that had been administered a ratiometric detection composition of the present disclosure:
   an initial fluorescence level of the reference dye at an initial time point; and
   an initial fluorescence level of the sensor dye at the initial time point;
b) determining a pH value at the initial time point, wherein a value of the initial fluorescence level of the sensor dye normalized to the initial fluorescence level of the reference dye is indicative of the pH value at the initial time point;
c) detecting in the subject:
   a subsequent fluorescence level of the reference dye at a subsequent time point; and
   a subsequent fluorescence level of the sensor dye at the subsequent time point; and
d) determining a pH value at the subsequent time point, wherein a value of the subsequent fluorescence level of the sensor dye normalized to the subsequent fluorescence level of the reference dye is indicative of the pH value at the subsequent time point,
e) optionally repeating steps c) to d).

In another aspect, the present disclosure includes a method of detection of a cation comprising
detecting in a subject that had been administered a ratiometric detection composition of the present disclosure:
   a fluorescence level of the reference dye; and
   a fluorescence level of the sensor dye; and
assessing a presence of the cation, wherein a positive value of the fluorescence level of the sensor dye normalized to the fluorescence level of the reference dye is indicative of the presence of the cation.

In another aspect, the present disclosure includes a method of measurement of a cation comprising
detecting in a subject that had been administered a ratiometric detection composition of the present disclosure:
   a fluorescence level of the reference dye; and
   a fluorescence level of the sensor dye;
determining a quantity of the cation, wherein a value of the fluorescence level of the sensor dye normalized to the fluorescence level of the reference dye is indicative of the quantity of the cation.

In another aspect, the present disclosure includes a method for monitoring of a cation comprising
a) detecting in a subject that had been administered a ratiometric detection composition of the present disclosure:
   an initial fluorescence level of the reference dye at an initial time point; and
   an initial fluorescence level of the sensor dye at the initial time point;
b) determining a quantity of the cation at the initial time point, wherein a value of the initial fluorescence level of the sensor dye normalized to the initial fluorescence level of the reference dye is indicative of the quantity of the cation at the initial time point;
c) detecting in the subject:
   a subsequent fluorescence level of the reference dye at a subsequent time point; and
   a subsequent fluorescence level of the sensor dye at the subsequent time point; and
d) determining a quantity of the cation at the subsequent time point, wherein a value of the subsequent fluorescence level of the sensor dye normalized to the subsequent fluorescence level of the reference dye is indicative of the quantity of the cation at the subsequent time point,
e) optionally repeating steps c) to d) and/or optionally comparing to the quantity of step b).

In some embodiments, the method further comprises administering topically in the subject the ratiometric detection composition, optionally the administering comprises the method of delivering a ratiometric detection composition of the present disclosure.

In some embodiments, the method is for ratiometric detection/measurement/monitoring of a plurality of analytes, and the ratiometric detection composition comprises a plurality of sensor dyes.

In some embodiments, the detecting of the fluorescence level of the reference dye and the detecting of the fluorescence level of the sensor dye are carried out with an imaging device, optionally a portable and/or wearable imaging device.

In some embodiments, the cation is Li⁺.

In some embodiments, the cation is Na⁺.

In some embodiments, the cation is K⁺.

In some embodiments, the cation is H⁺.

In another aspect, the present disclosure includes a kit for ratiometric detection/measurement/monitoring of an analyte comprising
a microneedle array of the present disclosure;
an applicator device for applying the microneedle array, optionally a spring-loaded applicator device; and
optionally one or more of instructions for use, and disinfecting wipe.

In some embodiments, the subject is a mammal. In some embodiments, the subject is human.

The following non-limiting examples are illustrative of the present disclosure:

### EXAMPLES

### General Methods

Poly(vinyl alcohol) (10 kDa) (PVA), dextran (from Leuconostoc spp. Mr˜ 6,000 Da), diisopropylethylamine (DIPEA), dimethyl sulfoxide (DMSO), N,N′-diphenylformamidine, disuccinimydal carbonate (DSC), iodomethane (Mel), malondialdehyde bis(phenylimine) monohydrochloride, nitromethane (CH₃NO₂), and 2,3,3-trimethylindolenine were purchased from Sigma-Aldrich (St. Louis, MO). Acetic anhydride (Ac₂O), acetonitrile (ACN), dichloromethane (DCM), diethyl ether (Et₂O), ethyl acetate (EtOAc), hydrochloric acid (HCl, 37 wt %, aq), hydrogen peroxide (30% v/v), methanol (MeOH), sodium chloride (NaCl), sodium sulfate (Na₂SO₄, anhydrous), potassium iodide (KI), and pyridine were purchased from Fisher Scientific (Waltham, MA). 6-bromohexanoic acid and iron sulfate heptahydrate (FeSO₄.7H₂O) were purchased from Acros Organics (Fair Lawn, NJ). Sephadex G-15 was purchased from GE Life Sciences (Pittsburgh, PA). Ultra-low molecular weight hyaluronic acid (<6 kDa) (ULMW HA) and super-low molecular weight hyaluronic acid (<50 kDa) were purchased from Lotioncrafter (Eastsound, WA). Poly(N-vinylpyrrolidone) (3.5-7 kDa) (PVP K-12) was generously provided by BASF (Ludwigshafen, Germany). Cyanine 7.5 NHS ester (Cy7.5-NHS) was purchased from Lumiprobe (Hunt Valley, MD). Methoxy-poly(ethylene glycol) amine (MeO-PEG-NH₂) (5 kDa) was purchased from JenKem Technologies (Plano, TX). 230-400 mesh silica was purchased from SiliCycle Inc. (Quebec, QC) and chromatographic separations were carried out using manual flash chromatography. Samples were concentrated in vacuo on the Rotavapor R-100 and heated using the B-100 heating bath (Büchi, Uster, Switzerland). ¹H-NMR spectra were recorded on a Varian MR400 NMR (Varian Inc., Palo Alto, CA). Chemical shifts are expressed in parts per million (ppm) and coupling constants are reported in hertz (Hz). Splitting patterns are indicated as: br=broad; s=singlet; d=doublet; dd=doublet of doublets; t=triplet; m=multiplet. LC-MS analyses were performed on a 6120 Quadrupole LC/MS provided by Agilent Technologies (Santa Clara, CA). Square pyramidal female MN molds of room temperature vulcanizing silicone, 10×10 array, 250 μm×250 μm×800 μm (W×L×H) were purchased from Micropoint Technologies Pte. Ltd. (Singapore). Excised pig skin samples were kindly provided by the Faculty of Veterinary Medicine, Universite de Montreal (Dr. Alexandre Thibodeau) and stored at −20° C. until use. Excised rat skin samples were kindly provided by the animal facilities of the Faculty of Pharmacy, Universite de Montreal, and stored at −20° C. until use.

### Example 1 Synthesis and Functionality of H-Cy5-PEG

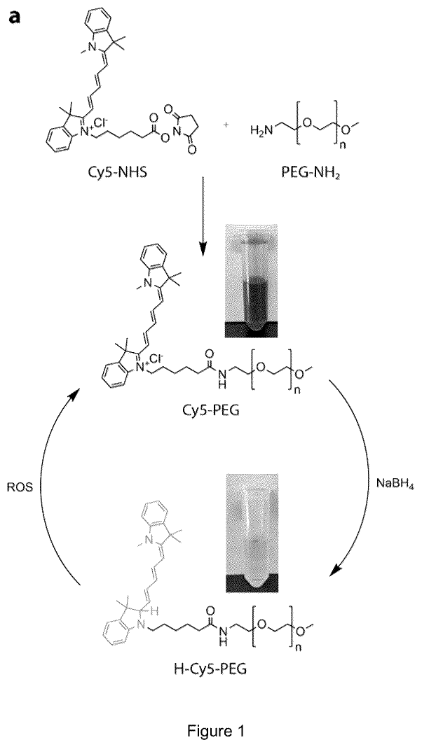

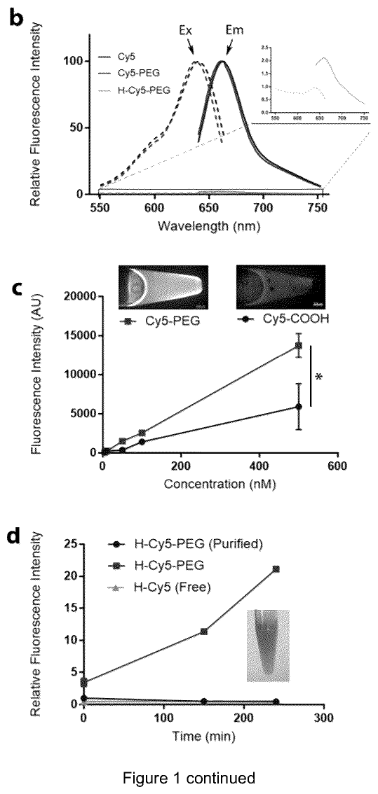

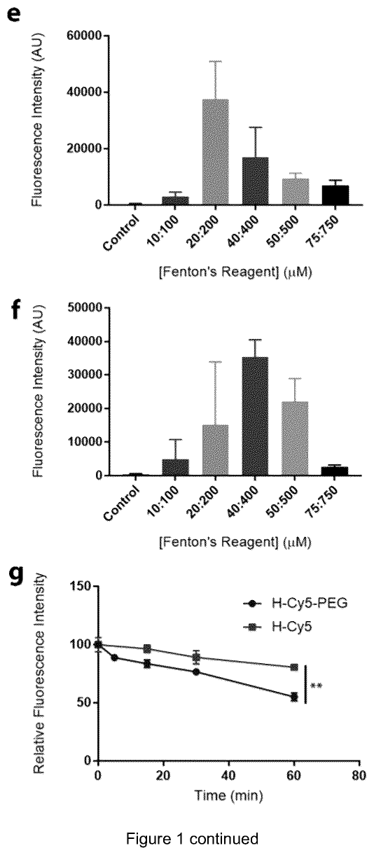

For this study, the dye Cyanine-5 (Cy5) was selected for sensing, owing to its relatively simple synthesis and high wavelength fluorescence (λₑₓ=646 nm; λₑₘ=662 nm), as well as the low toxicity and high biocompatibility of cyanine dyes,^{[47-50]} with a structural analogue being FDA approved for use in vivo.^{[51]} While the reduced hydrocyanine form of this dye (H-Cy5) had previously been described for in vitro and in vivo ROS sensing, it displays poor water solubility and, as a cyanine dye, it tends to form aggregates in aqueous solution,^{[52-54]} significantly quenching fluorescence. Further, as a small molecule, it is susceptible to uptake by cells of the dermis, where it would be unable to sense ROS in the extracellular ISF. To address this issue, Cy5 was PEGylated through amine coupling of an activated N-hydroxysuccinimide (NHS) ester to a 5 kDa methoxy-polyethylene glycol-amine (MeO-PEG-NH₂) polymer, resulting in a water-soluble extracellular Cy5 derivative (Cy5-PEG) (FIG. 1a,c).

The reduced hydrocyanine forms of Cy5 and Cy5-PEG (H-Cy5 and H-Cy5-PEG) were prepared using sodium borohydride (NaBH₄)—a mild reducing agent capable of selectively reducing the iminium cation of cyanine dyes,^{[40, 41]} disrupting the π-conjugation of the fluorophore and eliminating fluorescence. The reduction was confirmed by a color change from the characteristic blue color of Cy5 to a pale orange, and the disappearance of the fluorescence emission peak around 662 nm (FIG. 1b). As ROS-sensors are known to readily revert to their oxidized state due to light and environmental oxidants, the stability of both H-Cy5 and H-Cy5-PEG was assessed by diluting them in aqueous solutions (10 μM).^{[40]} While H-Cy5 displayed no significant increase in fluorescence over the course of 4 hours, H-Cy5-PEG was found to rapidly regain fluorescence, displaying a 20-fold increase within the same period of time (FIG. 1d). This was likely due to the presence of radical oxygen impurities—common in commercially available PEG polymers—resulting in rapid auto-oxidation of the sensor.^{[55]}

Using a previously described method for peroxide removal, a new H-Cy5-PEG derivative that displayed greatly improved stability in aqueous solutions, comparable to that of the unconjugated H-Cy5, was prepared (FIG. 1d).^{[56]} The excitation and emission spectra of the oxidized Cy5 and Cy5-PEG were also evaluated and found to be no shifts, suggesting that PEGylation causes no interference with the fluorescent ROS-sensing moiety of the dye (FIG. 1b).

The ROS sensitivities of H-Cy5 and H-Cy5-PEG were evaluated by mixing dilute solutions of each sensor (10 μM) with increasing concentrations of Fenton's reagent.^{[57,58] After mixing, the fluorescence of H-Cy}5 exposed to Fenton's reagent was found to increase up to 80-fold relative to a solution of the sensor in water, while H-Cy5-PEG displayed a maximum increase of over 60-fold (FIG. 1e,f), consistent with previously reported data showing increases in fluorescence intensity between 80 and 100-fold, depending on the hydrocyanine used.^{[41]} The signal intensity appeared to be dependent on the concentration of hydroxyl radical used. Concentrations below this range were likely below the detection limit of H-Cy5, while higher concentrations are known to promote the degradation of re-oxidized Cy5, explaining the decreased signal.^{[41]} However, it should be noted that the hydroxyl radicals produced by Fenton's reagent are difficult to quantify due to their high reactivity and short half-lives, in the range of nanoseconds.^{[59-61]} This re-oxidation appeared to occur nearly instantaneously, with the strongest signal observed immediately after mixing, followed by a gradual decay, with roughly 80% of the initial signal remaining after 1 hour for H-Cy5, and roughly 60% remaining for H-Cy5-PEG, likely due to the gradual degradation of Cy5 following re-oxidation (FIG. 1g).^{[41]}

### Example 2 Selection and Characterization of MN Polymers

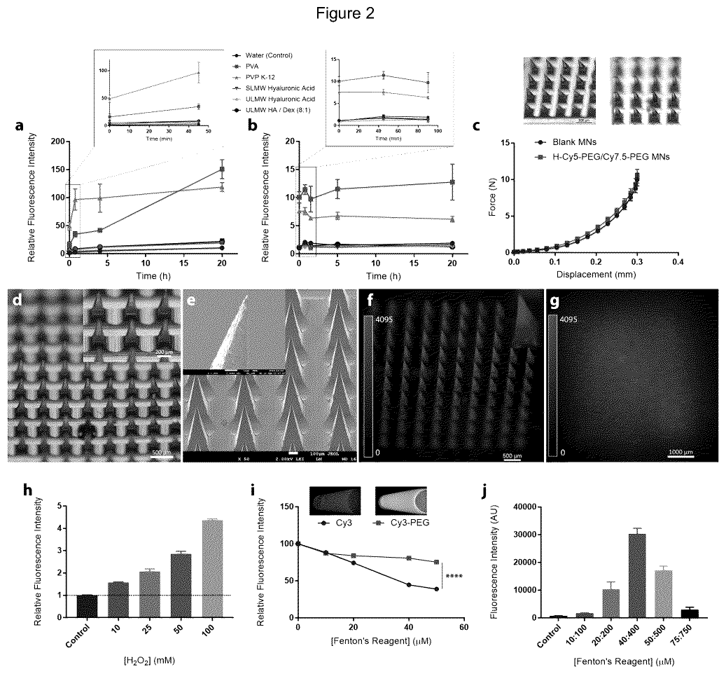

Following confirmation of the ability of the water-soluble PEGylated sensor to detect ROS, its compatibility with a range of natural and synthetic MN polymers was assessed and compared to free H-Cy5, in order to select the optimal material for MN fabrication. To test this, sensors were diluted (10 μM) in concentrated solutions of each polymer and the fluorescence was monitored as a function of time. Both H-Cy5 and H-Cy5-PEG were rapidly oxidized in the presence of PVP K-12 and PVA, with H-Cy5 displaying up to 100-fold greater fluorescence relative to a solution in water, likely due to the presence of ROS-containing impurities in the polymers (FIG. 2a).^{[62,63]} While H-Cy5-PEG also appeared to be oxidized by both PVP and PVA, the effect was much less pronounced than with H-Cy5, with only a 5-to-10-fold increase in fluorescence observed (FIG. 2b), which could be due to the solubilization effects of PVP and PVA on the poorly soluble H-Cy5, increasing the apparent fluorescence of the solution, while H-Cy5-PEG was already fully solubilized.^{[64,65]}

Other polymers showed more promising results, with ultra-low molecular weight hyaluronic acid (Mw<6,000 Da) (ULMW HA) and super-low molecular weight hyaluronic acid (M_{w}<50,000 Da) (SLMW HA) displaying no oxidizing effect. A carbohydrate-based biopolymer, HA is an important component of the extracellular matrix (ECM) of the skin and has previously been used to prepare dissolving polymeric MNs.^{[66]} Specifically, an 8:1 blend of ULMW HA and dextran was found to produce MNs with sharp tips and excellent mechanical properties. This blend of polymers was also tested with H-Cy5 and H-Cy5-PEG and was found to have no oxidizing effect (FIG. 2a,b).

### Example 3 Characterization of Dissolving Polymeric MNs

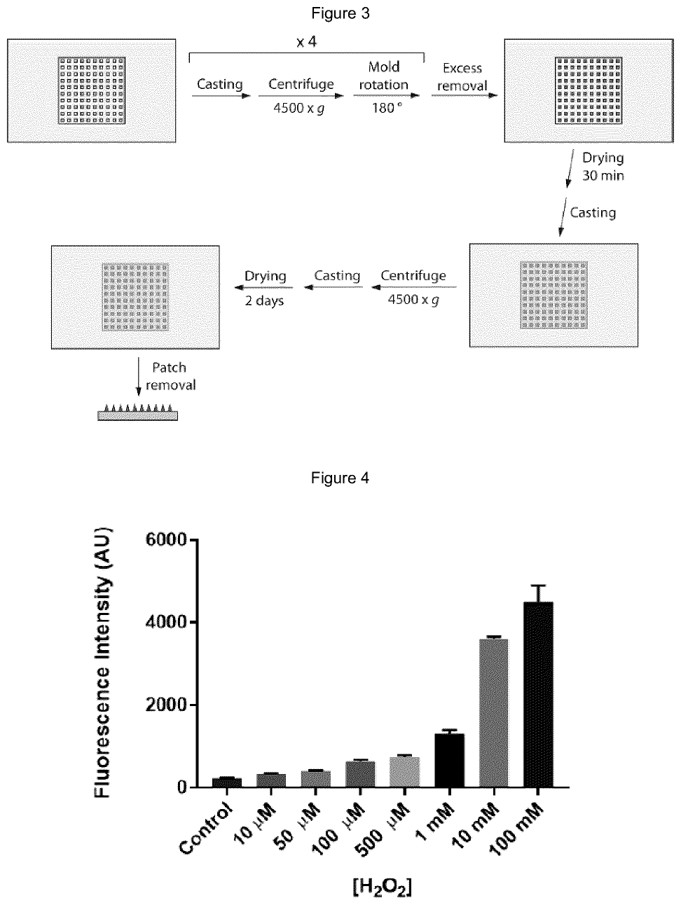

To ensure that the formulation was suitable for piercing the skin, MNs were prepared from an 8:1 blend of ULMW HA and dextran through a solvent casting method in PDMS molds (FIG. 3), and their mechanical properties were assessed. The resulting MNs were found to have an average failure force of 10±1 N, sufficient to pierce human skin without deformation, with the most common mode of failure being 90° deflection without fracture of the MN tip (FIG. 2c).^{[11]} The effect of the incorporation of PEGylated sensor on mechanical strength was studied by preparing MNs from the same polymer blend with the addition of H-Cy5-PEG at 250 μM, the highest concentration of sensor used in this study. The average failure force of the resulting MNs was found to be 10±2 N, not significantly different from those made without H-Cy5-PEG, indicating that the inclusion of relevant amounts of H-Cy5-PEG within the solid MN structure would not compromise the ability of the MNs to pierce skin.

Using bright-field microscopy, the resulting MNs were shown to form a 10×10 array of uniform pyramidal projections, with lengths of 604±17 μm and widths of 249±7 μm×249±7 μm at the base (FIG. 2d). While the measured base width was in line with the dimensions of the molds (250 μm×250 μm), the lengths were significantly lower than the stated lengths of the molds (800 μm). This was likely due to contraction of the polymer due to water loss during the drying process. Scanning electron microscopy revealed that the MNs consistently formed sharp tips with diameters significantly below 10 μm, and sufficient to pierce the skin (FIG. 2e).^{[68-70]}

In order to optimize delivery of the fluorescent microneedle tattoo to the skin, MNs were prepared using a previously described protocol, localizing the probes selectively within the MNs tips (FIG. 2f).^{[9,61,71]} To ensure their ability to deliver sensors to the skin, MNs containing oxidized Cy5-PEG were applied to a rat shaved skin using a spring-loaded applicator. After 2 minutes of application, a fluorescent tattoo could clearly be observed at the location of application using fluorescence stereomicroscopy, indicating successful skin penetration and release of dye (FIG. 2g). Diffusion of the dye could also be observed, highlighting the solubility of Cy5-PEG relative to free Cy5, which did not appear to significantly diffuse from the application site.

### Example 4 Validation of H-Cy5-PEG ROS-Sensing Activity after Drying in MNs

After confirming the ability of MNs to deliver a PEGylated dye to the skin, the activity of the sensor (H-Cy5-PEG) was investigated after drying within the polymer matrix of the MNs. To test this, H-Cy5-PEG MNs prepared from ULMW HA and dextran were dissolved in increasing concentrations of H₂O₂, and a fluorescence increase of approximately 5-fold was observed (FIG. 2h). The significantly smaller increase in fluorescence observed is likely due to two factors. First, during preparation and drying of the MNs, H-Cy5-PEG was observed to become partially oxidized, likely due to remaining in aqueous solution and exposure to atmospheric oxygen. Additionally, H-Cy5-PEG was observed to be less sensitive to H₂O₂ alone, with a roughly 20-fold increase in fluorescence observed in solution, whereas the increase was between 60 and 80-fold for Fenton's reagent (FIG. 4). Nonetheless, the concentration-dependent fluorescence increase observed demonstrated that the sensing activity of H-Cy5-PEG was retained after drying.

### Example 5 Selection and Validation of Fluorescent Reference Dye

To cope with the heterogeneity of the skin's surface—which can result in differences in dye delivery between applications—a reference dye was incorporated into the MNs to act as an internal standard, accounting for this variability in the amount of dye delivered. The dye selected for this purpose was Cyanine-3 (Cy3)—another cyanine dye related to Cy5. Cy3 was chosen due to its similar synthesis and structure to Cy5, allowing PEGylation at the same site, resulting in a dye with similar physicochemical properties to Cy5-PEG, but displaying maximal fluorescence emission at a wavelength ˜100 nm lower (λₑₓ=555 nm; λₑₘ=570 nm). This was validated by experimentally determining the log P values of Cy5-PEG and Cy3-PEG, which were found to be −1.22±0.07 and −1.03±0.02 respectively, indicating dyes of similar hydrophilicity as well as size. Further, Cy3-PEG was found to have good stability when exposed to ROS, retaining over 75% of its signal at high radical concentration (50 μM/500 μM of FeSO₄:H₂O₂); a marked improvement over free Cy3, which retained only 40% of its initial signal when exposed to this level of ROS (FIG. 2i). The re-oxidation of H-Cy5-PEG was also investigated in the presence of Cy3-PEG, to ensure the absence of interference with the reaction. The re-oxidation proceeded as normal, with H-Cy5-PEG reaching fluorescence intensity values comparable to when no Cy3-PEG was present, highlighting the suitability of Cy3-PEG as a reference dye (FIG. 2j).

Following the selection of Cy3-PEG as a reference dye, the concentrations of both Cy5-PEG and Cy3-PEG were optimized for delivery to the skin. MNs containing concentrations of either Cy5-PEG or Cy3-PEG between 50 μM and 250 μM were prepared and applied to excised rat skin, and the fluorescence of the resulting tattoos was measured using a fluorescence stereomicroscope (FIG. 5a,b). A clear increase in fluorescence was observed for both dyes between 50 μM and 150 μM, however a sharp decrease was noted at 250 μM, likely due to aggregation and quenching of the fluorophores. Accordingly, a maximum concentration of 150 μM was used for all subsequent tests.

Following this, MN tattoos with concentrations of 50 μM, 100 μM, and 150 μM of both Cy5-PEG and Cy3-PEG were applied to the shaved skin of living rats, to ensure that the delivery and diffusion of both dyes was similar in vivo. MNs containing 150 μM of free Cy5 and Cy3 were also applied, to compare the diffusion and delivery of more hydrophobic molecules. When the rat skin was examined by fluorescence stereomicroscopy (several hours after application), both PEGylated dyes were clearly diffused in the skin, appearing as areas of uniform fluorescence, in contrast to the distinct MN pattern with minimal diffusion resulting from the free dyes (FIG. 5c-h).

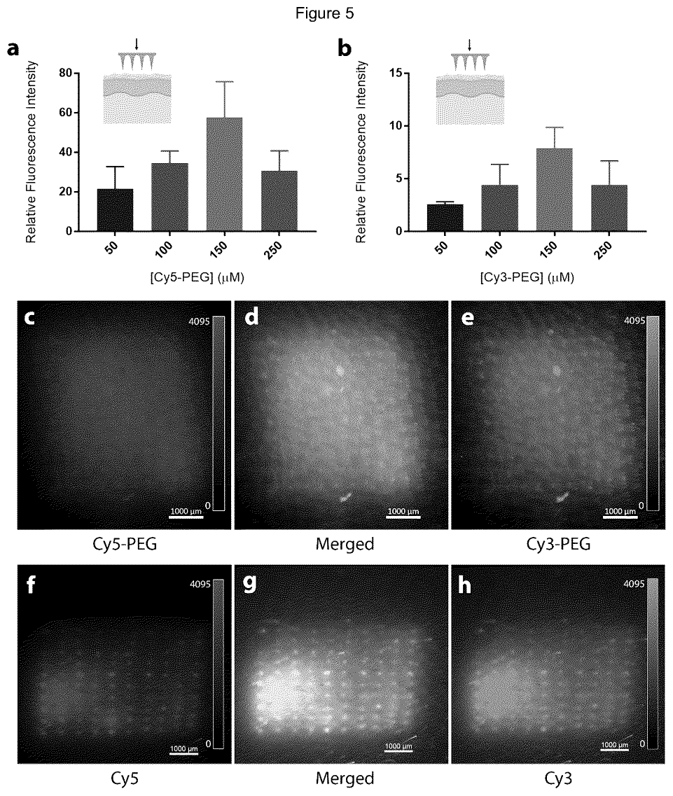

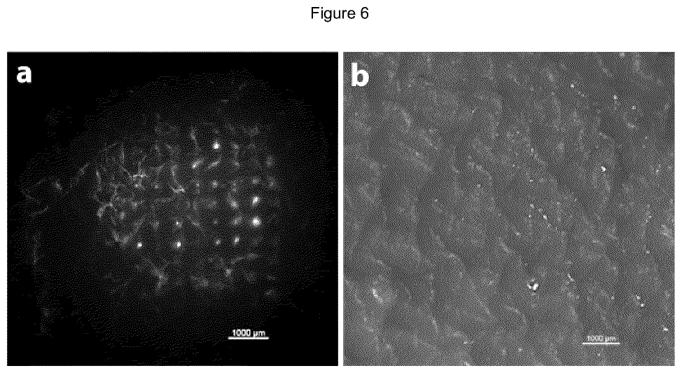

The skin was also examined under bright-field microscopy, where no evidence of the microneedle tattoo (i.e. color, pattern) could be seen, in spite of the strong fluorescent signal (FIG. 6). This is likely due to the small quantity of dye delivered to the dermis by the microneedle tattoo—only 1.5±0.5 μg of each dye (or a total of 3±1 μg) is delivered per application for 150 μM MNs. The colocalization of Cy5-PEG and Cy3-PEG fluorescence was also quantified in the skin using the Pearson correlation coefficient, resulting in a value of 0.98±0.01 and indicating that both dyes behave comparably in the skin.^{[86]} Finally, it should be noted that these microneedle tattoos are likely to be highly transient in nature, with previous studies demonstrating that PEGylated cyanines—particularly those with low molecular weight—readily diffuse within the ISF before being drained through the lymphatic vasculature.^{[17,19,73]} This could prevent bioaccumulation at the site of application and allow clearance of fluorescent sensors following measurements.

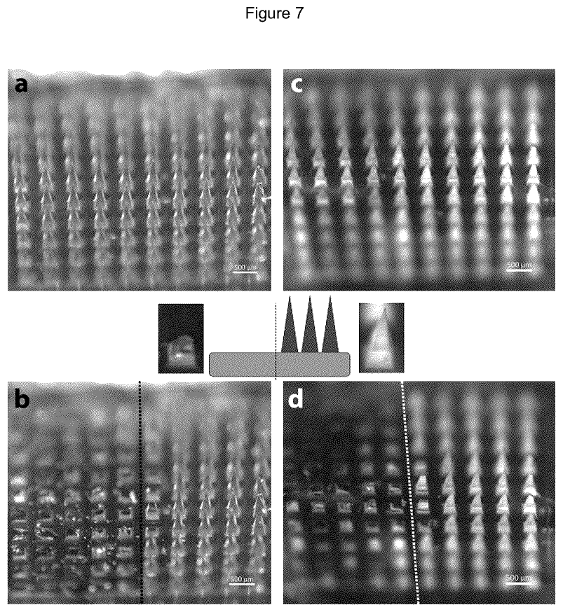

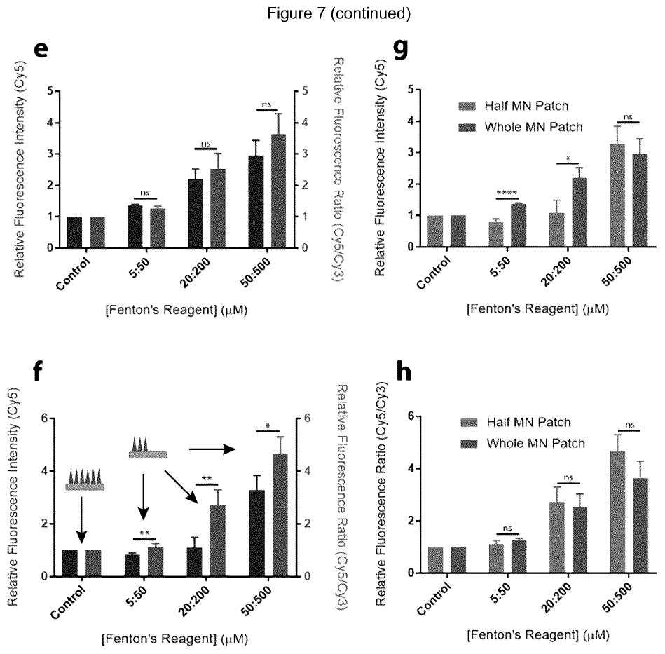

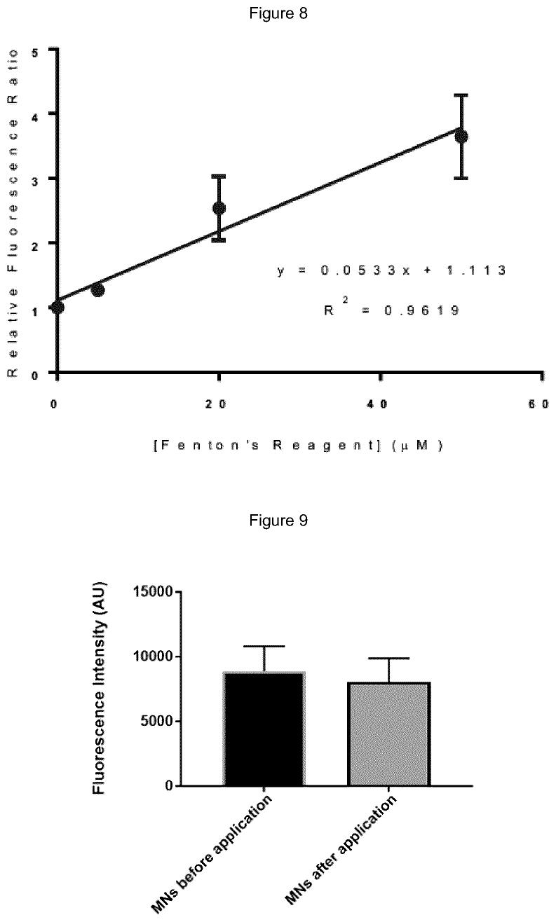

The ability of Cy3-PEG to correct for the amount of sensor delivered was supported by tests conducted in agarose gels, used as a skin-simulating medium.^{[74,75]} A 3% w/w agarose gel was chosen based on its viscoelastic properties comparable to human skin, and the ability to easily control the level of ROS present by adjusting the concentration of Fenton's reagent within the gels. MNs containing both H-Cy5-PEG and Cy3-PEG were prepared and applied to the gels, and the resulting fluorescent signal was quantified by two-dimensional fluorescence scanning. Clear signals at both Cy5 and Cy3 wavelengths could be observed, and an increase in H-Cy5-PEG fluorescence intensity between 3-fold and 4-fold was observed in gels containing high concentrations of ROS relative to control gels (FIG. 7e). No significant difference was observed when calculating the ratio of H-Cy5-PEG fluorescence to the fluorescence of Cy3-PEG, likely due to the delivery of similar amounts of dye in each test. Notably, the relationship between the concentration of Fenton's reagent and the fluorescence ratio of the microneedle tattoo was found to be linear (R² 0.9619, FIG. 8). To validate the normalization approach, in a subsequent test, MNs were applied to control gels, containing no source of ROS, without modification (FIG. 7a,c).

For gels containing Fenton's reagent (a source of ROS), half of the MN tips were removed before application (FIG. 7b,d), resulting in the delivery of roughly half the total amount of sensor, and simulating a situation of incomplete application of MNs to skin. As expected, the relative fluorescence intensity of H-Cy5-PEG alone was reduced, even in the presence of ROS, due to the lower amount of H-Cy5-PEG delivered. This can be seen most clearly in the gels with Fenton's reagent concentrations of 5 μM/50 μM and 20 μM/200 μM, where H-Cy5-PEG fluorescence intensity was lower than in the control gel (FIG. 7f). However, this difference is corrected by calculating the ratio of H-Cy5-PEG fluorescence to that of the reference dye Cy3-PEG, restoring normal ROS-sensitivity of around 3-fold to 4-fold. This demonstrated that the inclusion of a reference dye, such as Cy3-PEG, allows the tattoo to function as a ratiometric sensor, effectively correcting for any variability in sensor delivery due to differences in application or skin topography. This can also be illustrated by comparing the fluorescence of MN tattoos delivered to gels by whole MN patches and MN patches with half of the tips removed. When measuring the relative fluorescence intensity of H-Cy5-PEG alone, a significant difference is observed depending on the amount of dye delivered, while no such difference is observed when calculating the ratio of fluorescence between H-Cy5-PEG and Cy3-PEG (FIG. 7g,h).

Notably, the relative increase in fluorescence observed in agarose gels for the MN tattoo was lower than in solution. The primary reasons for this are likely the oxidation of H-Cy5-PEG during MN preparation and the relatively small quantity of dye delivered by the MNs (FIG. 9). It has been observed that H-Cy5-PEG is relatively sensitive to oxidation by factors other than ROS, notably atmospheric oxygen and light, particularly when stored at high concentrations in aqueous solution. As such, despite care being taken during MN preparation, such as limiting exposure of the solutions to air and light, a certain amount of oxidation cannot be avoided during the MN drying process.

### Example 6 Sensing Ability of H-Cy5-PEG/Cy7.5-PEG Microneedle Tattoo in a Human-Based Atopic Skin Model

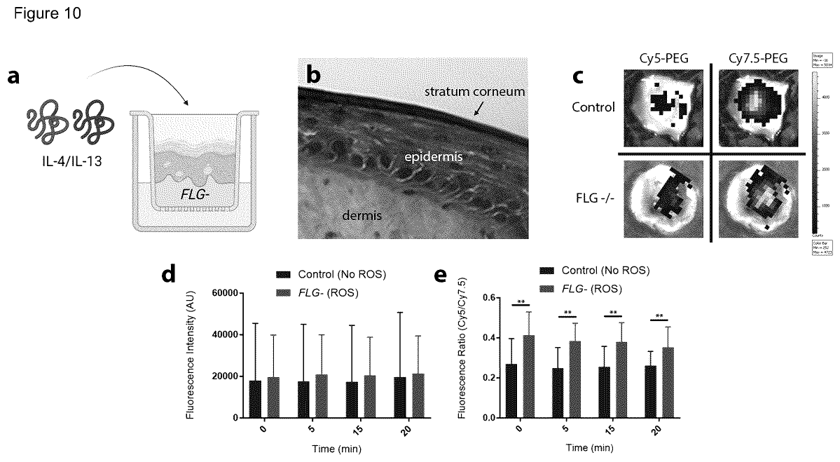

To provide the proof of concept for our approach and to validate the potential use of a medical tattoo in a biological system, the sensing properties of the generated MNs were tested in human-based inflammatory skin model that closely emulates characteristics of atopic skin in vitro^{[76]} and has been previously verified for the presence of disease-relevant ROS.^{[77]} This model has been extensively characterized in previous work showing excellent correlation with atopic skin in vivo.^{[76,78,79]} The skin model is based on a filaggrin knockdown, as filaggrin mutations are a major pre-disposing factor for AD.^{[80]} To emulate the inflammatory conditions, the AD-characteristic cytokines IL-4 and IL-13 have been added (FIG. 10a). This system could thereby serve as a proof-of-concept for effective real-time monitoring of skin inflammation, paving the way for more effective tracking of disease progression and presenting opportunities for treatment adaptation. Notably, owing to the relatively high tissue autofluorescence observed at 570 nm (the emission wavelength of Cy3), an alternate reference dye was selected, namely Cyanine-7.5 (Cy7.5).[^{21,41,42]} Cy7.5 was selected based on its structural similarity to Cy5 and other cyanines, displaying similar physicochemical properties and allowing PEGylation in the same manner as Cy5, as well as its NIR fluorescence (λₑₓ=788 nm; λₑₘ=808 nm), optimal for visibility in the skin. MNs containing the sensor (H-Cy5-PEG) and the new reference dye (Cy7.5-PEG) were tested in inflamed skin models (FIG. 10b). The fluorescence of the resulting microneedle tattoo was measured intermittently over 20 minutes, and clear signals could be observed at both the H-Cy5-PEG and Cy7.5-PEG wavelengths (FIG. 10c).

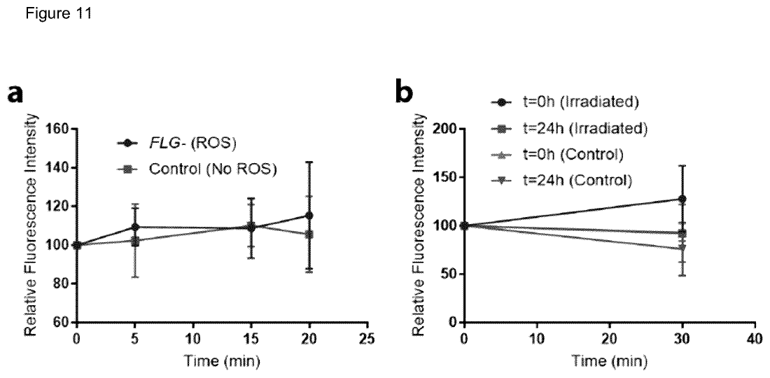

When examining the H-Cy5-PEG fluorescence alone, no difference between the control and inflamed skin models could be seen, with the signal displaying high variability, potentially due to variations in the quantity of the delivered sensor (FIG. 10d). However, by analyzing both the H-Cy5-PEG and Cy7.5-PEG wavelengths, the ratio between the H-Cy5-PEG and Cy7.5-PEG signals was calculated. Both signals were found to be stable over time, and no variance of the fluorescence ratio was observed, highlighting the suitability of Cy7.5-PEG as an inert reference dye for this application (FIG. 11a). When comparing the fluorescence ratio between normal skin models and the inflammatory FLG-deficient models, the latter showed a statistically significant increase of ROS production, as derived from a stronger fluorescent sensor signal relative to the reference dye (FIG. 10e). This demonstrated that, in spite of reduced sensitivity likely owing to the effects of environmental oxidants, H-Cy5-PEG remained sensitive to re-oxidation after skin delivery, and importantly, that the microneedle tattoo was capable of detecting ROS/inflamed states in human-based tissue.

### Example 7 Characterization and Ex Vivo Validation of Portable Fluorescence Reader

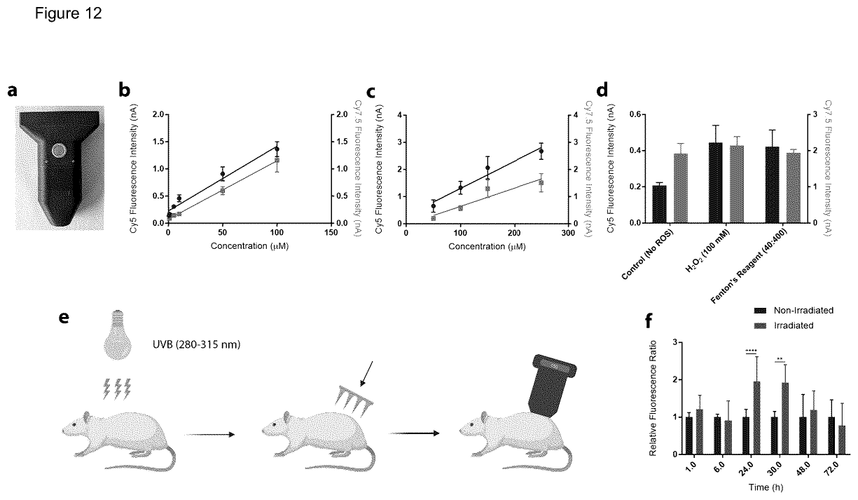

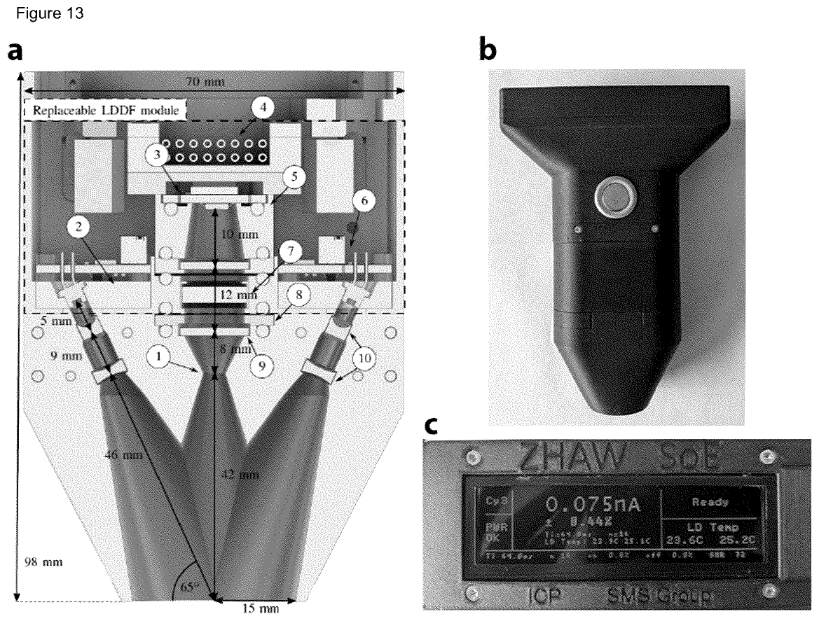

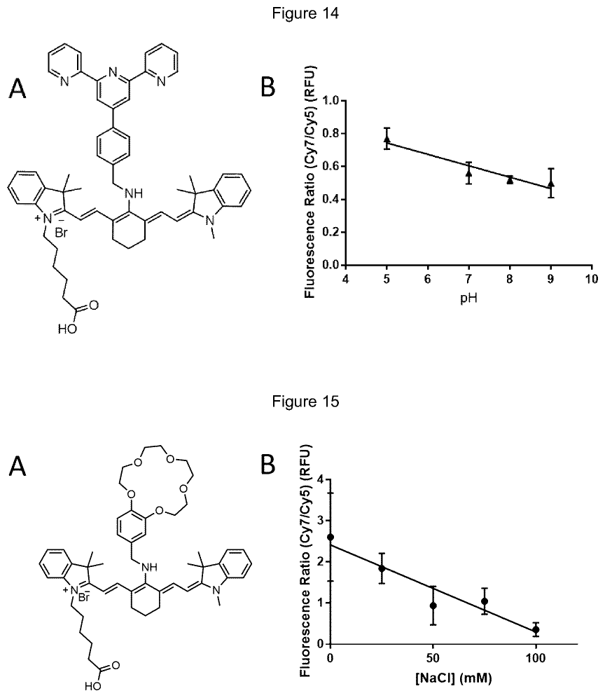

When developing precision health monitoring systems, a key concern is the possibility of portable monitoring, as this could allow translation of the technology to an at-home setting and reduce the need to travel to a facility with specialized monitoring equipment. Indeed, our previous work described the use of a custom-built portable NIR fluorescence detector for the monitoring of lymphatic drainage in mice, with the device displaying comparable results to a traditional IVIS imaging system.^{[17]} With this in mind, we sought to test the compatibility of our ROS-sensing microneedle tattoo with an improved portable fluorescence reader, modified to allow measurement of multiple wavelengths—notably that of Cy5 (λₑₘ=662 nm) (FIG. 12a) (FIG. 13b). This new portable fluorescence reader was first validated using solutions of Cy5 and Cy7.5, where a linear dynamic range of 1-150 μM was observed (FIG. 12b). This was further confirmed by using the device to measure the fluorescence intensity of Cy5-PEG and Cy7.5-PEG microneedle tattoos of increasing concentrations delivered to porcine skin. This test also showed good linearity at dye concentrations ranging from 50 μM to 250 μM, indicating that the portable fluorescence reader was well suited for measuring the amounts of dye delivered for ROS-sensing (FIG. 12c).

Following this validation, the device was tested to determine whether it could be used to observe ROS-sensing by a H-Cy5-PEG/Cy7.5-PEG microneedle tattoo in the skin, using tests conducted in porcine skin. Microneedle tattoos containing both the sensor and reference dye were delivered ex vivo to excised porcine skin that had been treated with either distilled water (control), or a source of ROS—either H₂O₂ (100 mM) or Fenton's reagent (40 μM/400 μM). While the fluorescence of the microneedle tattoo at Cy7.5-PEG wavelengths was unaffected by the presence of ROS, the fluorescence intensity of H-Cy5-PEG was doubled in the skin treated with either ROS source, indicating that the device possessed sufficient sensitivity for the detection of elevated ROS levels in the skin (FIG. 12d).

### Example 8 In Vivo Sensing of UV Irradiation-Induced ROS in Rat Skin

In addition to their association with diseases such as atopic dermatitis, dermal ROS have associated with exposure to UV light and ensuing skin inflammation. Specifically, this is proposed to occur via a neutrophil-mediated mechanism, in which exposure to UVB radiation (280-320 nm) induces a release of cytokines (i.e. IL-8 and TNF-α) in the skin, which triggers the recruitment of neutrophils to the dermis.^{[29,30]} In the dermis, these neutrophils generate ROS through the action of enzymes including myeloperoxidase and NADPH oxidase, resulting in cell and tissue damage, with maximal neutrophil levels generally recorded between 18-32 h after UV exposure.^{[26-28]} Accordingly, the H-Cy5-PEG/Cy7.5-PEG microneedle tattoo was tested in vivo using a rat model of UVB-induced dermal inflammation.

Briefly, rats were exposed UVB light, after which MNs were applied to their backs at times ranging from 0-72 h post-irradiation, and the fluorescence ratio (H-Cy5-PEG/Cy7.5-PEG) of the resulting microneedle tattoo was measured using the previously described portable fluorescence reader and compared to a control animal (FIG. 12e). Immediately following UVB irradiation, no significant difference in microneedle tattoo fluorescence was seen, with the irradiated and non-irradiated rats displaying comparable fluorescence at both H-Cy5-PEG and Cy7.5-PEG wavelengths. However, at t=24 h and t=30 h, it was observed that the fluorescence ratio (H-Cy5-PEG/Cy7.5-PEG) was significantly greater in the UV-irradiated rats than in the control rats, with this difference largely disappearing at later time points (48 h and 72 h) (FIG. 12f). This result was consistent with the time frame of recruitment of neutrophils to the dermis and the associated ROS production), suggesting that the microneedle tattoo was capable of successfully detecting elevated levels of UV-induced dermal ROS in vivo. Finally, when examining the Cy7.5-PEG fluorescence intensity of the microneedle tattoo 30 minutes after application (at both t=0 h and t=24 h after irradiation), no significant decrease was observed, suggesting that the reference dye stable and suitable for in vivo applications (FIG. 11b).

The intradermal delivery of functional sensors for diagnostic and monitoring purposes is a burgeoning field with a wide variety of potential applications. This proof-of-concept study sought to demonstrate that an active sensor can be delivered intradermally using MNs, acting as a ratiometric fluorescent microneedle tattoo and to allow the monitoring of (patho-)physiological conditions. It was found that by selecting an appropriate dye and optimizing a compatible polymer formulation, it was possible to retain sensing ability after the formation and drying of the MNs. Further, it was demonstrated that the sensor displayed a significant increase in fluorescence in the presence of ROS when delivered to both a human-based skin disease model emulating atopic dermatitis in vitro, and an in vivo rat model of UV-induced dermal inflammation. Importantly, this work highlights—in the form of a ratiometric microneedle tattoo—a potentially universal method of normalizing the signal of dermal fluorescent sensors, ultimately paving the way for the quantification and monitoring of a wide range of health information. Finally, it should be noted that no precise quantification of ROS levels was attempted in vivo or in tissue models. This was primarily a function of the transient nature and short half-lives of biological ROS, as the precise levels present in the models used in this study have not been thoroughly characterized by other methods. Indeed, for future applications, such as the monitoring of more stable biological analytes, ex vivo and in vivo quantification will be an important goal.

Ultimately, the concept of a MN-delivered ratiometric fluorescent microneedle tattoo constitutes an exciting prospect for precision health monitoring, and the system described here constitutes the first example of a functional sensor delivered via this method in a biologically relevant model. Many of the challenges faced by the system described in this article are specific to ROS-sensing, namely the tendency of H-Cy5-PEG to be oxidized during MN preparation, as well as the difficulty of directly quantifying ROS concentration, and could be avoided in future applications of this technology. By selecting appropriate sensors, with high brightness and stability, this technique could be applied to the sensing and monitoring of a variety of biologically relevant analytes, and the potential of this system could be more fully realized.

### Example 9 Experimental Details

### Synthesis of Cy5

### 1,2,3,3-Tetramethyl-3H-indol-1-ium iodide (S1)

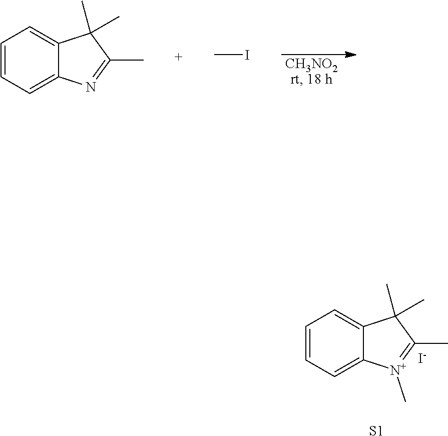

S1 was synthesized using a previously described procedure.^{[81]} Briefly, 2,3,3-trimethylindolenine (2.52 mL, 15.7 mmol, 1 eq) was diluted in 9 mL CH₃NO₂. Mel (1.95 mL, 31.4 mmol, 2 eq) was added dropwise and the reaction mixture was stirred at room temperature for 20 h under argon. The crude product was precipitated using 60 mL of Et₂O, and the resulting purple solid was collected by vacuum filtration and washed 3× with 15 mL of Et₂O. A pale purple solid was obtained (4.17 g, 13.8 mmol, 88% yield). ¹H NMR (Varian, 400 MHz, DMSO-d6): δ=7.91-7.88 (m, 1H), 7.82-7.80 (m, 1H), 7.63-7.57 (m, 2H), 3.96 (s, 3H), 2.75 (s, 3H), 1.51 (s, 6H) ppm.

### 1-(5-Carboxylpentyl)-2,3,3-trimethyl-3H-indol-1-ium iodide (S2)

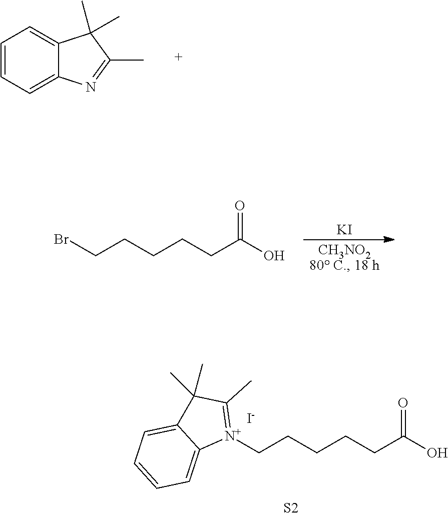

S2 was synthesized by adapting a previously described procedure.^{[82]} Briefly, 2,3,3-trimethylindolenine (1.60 mL, 10 mmol, 1 eq) and 6-bromohexanoic acid (1.95 g, 10 mmol, 1 eq) were dissolved in 6 mL ACN. KI (1.66 g, 10 mmol, 1 eq) was added and the reaction mixture was refluxed at 85° C. for 16 h under argon. The mixture was filtered, concentrated under reduced pressure, and the crude product was precipitated by adding 40 mL of 1:1 EtOAc/DCM and cooling to −20° C. The resulting purple solid was collected by vacuum filtration and washed 3× with 5 mL of 1:1 EtOAc/DCM. A light purple solid was obtained (2.38 g, 5.9 mmol, 59% yield). ¹H NMR (Varian, 400 MHz, DMSO-d6): δ=12.00 (br. s, 1H), 7.99-7.94 (m, 1H), 7.85-7.81 (m, 1H), 7.63-7.58 (m, 2H), 4.44 (t, J=7.8 Hz, 2H), 2.84 (s, 3H), 2.21 (t, J=7.2 Hz, 2H), 1.83 (m, 2H), 1.58-1.50 (m, 8H), 1.45-1.39 (m, 2H) ppm.

### Cyanine-5 (Cy5)

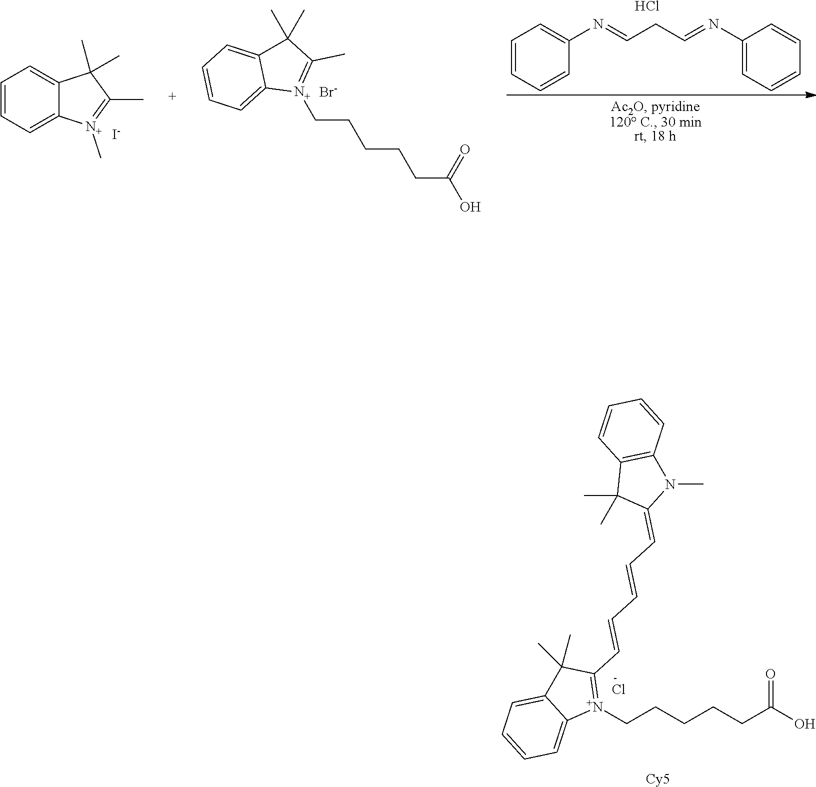

Cy5 was synthesized by adapting a previously described procedure.^{[81]} Briefly, S2 (734 mg, 1.8 mmol, 1 eq) and malondialdehyde bis(phenylimine) monohydrochloride (521 mg, 2.0 mmol, 1.1 eq) were dissolved in 6 mL of Ac₂O and the mixture was refluxed at 120° C. for 30 min under argon, then cooled to room temperature. S1 (660 mg, 2.2 mmol, 1.2 eq) was suspended in 6 mL of dry pyridine and added to the reaction mixture. The reaction was stirred for 18 h at room temperature, with protection from light. The solvents were removed under reduced pressure at 60° C. The resulting blue solid was dissolved in 20 mL DCM and washed 3× with 40 mL dH₂O, 1× with 40 mL of 1 M HCl_{(aq)} and 1× with 40 mL of sat. NaCl. The organic layer was dried over Na₂SO₄ and concentrated under reduced pressure. The crude mixture was purified by column chromatography, eluting with a gradient of 0-10% MeOH in DCM. Cy5 was obtained as a metallic red-blue solid (486 mg, 0.93 mmol, 51% yield).

¹H NMR (Varian, 400 MHz, DMSO-d6): δ=12.11 (br. s, 1H), 8.31 (t, J=13.1 Hz, 2H), 7.60 (d, J=7.4 Hz, 2H), 7.41-7.35 (m, 4H), 7.26-7.19 (m, 2H), 6.55 (t, J=12.3 Hz, 1H), 6.27 (dd, J=17.0 Hz, 13.9 Hz, 2H), 4.07 (t, J=7.2 Hz, 2H), 3.58 (s, 3H), 2.18 (t, J=7.2 Hz, 2H), 1.76-1.61 (m, 14H), 1.58-1.49 (m, 2H), 1.40-1.32 (m, 2H) ppm.

### Synthesis of Cy5-PEG

### Cyanine-5 N-Hydroxysuccinimide ester (Cy5-NHS)

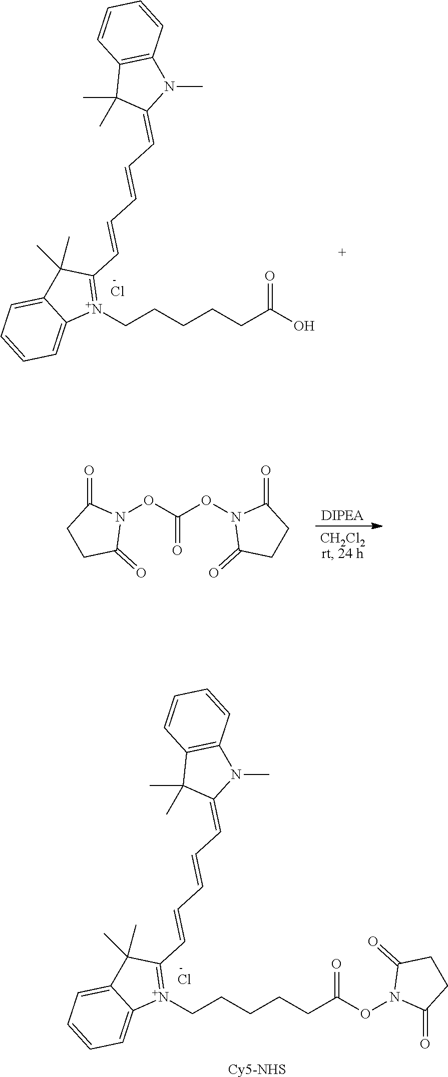

Cy5 was activated as Cy5-NHS by adapting a previously described procedure.^{[81]} Briefly, Cy5 (486 mg, 0.93 mmol, 1 eq) was dissolved in 10 mL of dry DCM. DIPEA (326 μL, 1.87 mmol, 2 eq) and DSC (536 mg, 2.06 mmol, 2.2 eq) were added and the reaction was stirred for 24 h at room temperature, with protection from light. The mixture was diluted with an additional 10 mL DCM and washed 4× with 40 mL dH₂O, 1× with 40 mL of 1 M HCl_{(aq)}, and 1× with 40 mL of sat. NaCl. The organic layer was dried over Na₂SO₄ and further dried under reduced pressure to give Cy5-NHS as a metallic red-blue solid (544 mg, 0.88 mmol, 95% yield). The purity of the product was confirmed by HPLC-MS analysis. ¹H NMR (Varian, 400 MHz, DMSO-d6): δ=8.32 (t, J=13.1 Hz, 2H), 7.60 (d, J=7.4 Hz, 2H), 7.41-7.35 (m, 4H), 7.26-7.19 (m, 2H), 6.54 (t, J=12.3 Hz, 1H), 6.26 (t, J=14.1 Hz, 2H), 4.07 (t, J=7.2 Hz, 2H), 3.58 (s, 3H), 2.80 (s, 4H), 2.69-2.65 (m, 2H), 1.76-1.61 (m, 16H), 1.50-1.45 (m, 2H) ppm.

### Purification of PEG-NH2

Purification of PEG-NH2 was adapted from a previously described procedure.^{[55]} Briefly, MeO-PEG-NH₂ (M_{w}=5,000 g/mol) (60 mg, 12 μmol) was dissolved in 1 mL of milli-Q H₂O and cooled to −80° C. This solution was lyophilized at 40 mT for 48 h, yielding a fluffy white solid, which was stored at −20° C. under nitrogen.

### Cyanine-5-PEG (Cy5-PEG)

Cy5-PEG was synthesized by adapting a previously described procedure.^{[73]} Briefly, Cy5-NHS (7.4 mg, 12 μmol, 2 eq) and MeO-PEG-NH₂ (M_{w}=5,000 g/mol) (30 mg, 6 μmol, 1 eq) were dissolved in 1 mL of anhydrous DMSO, and the mixture was stirred for 18 h, with protection from light. The mixture was then diluted in 20 mL of milli-Q H₂O and lyophilized. The resulting residue was dissolved in 1 mL of 180 mM NaCl solution and purified on G-15 Sephadex. The resulting fractions were combined and lyophilized, giving Cy5-PEG as a blue solid (28 mg, 5.6 μmol, 86% yield). The purity of the product was confirmed by HPLC-MS analysis.

### Synthesis of CY3-PEG

### Cyanine-3 (Cy3)

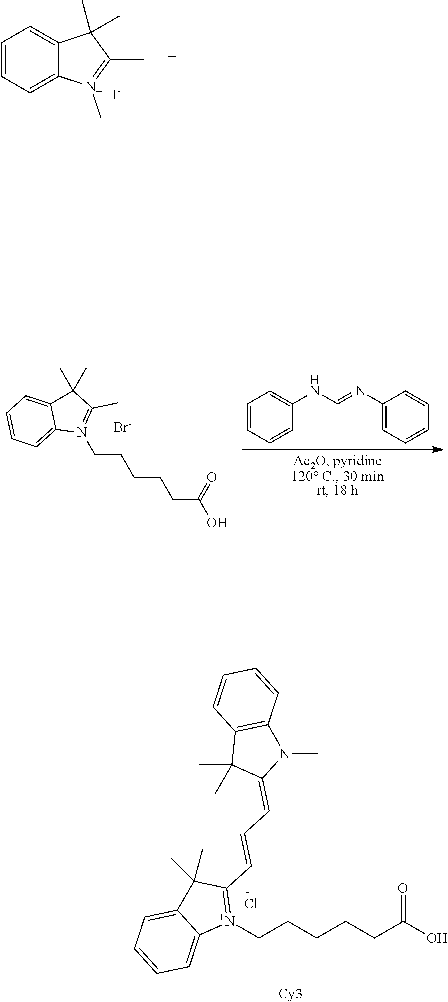

Cy3 was synthesized by adapting a previously described procedure.^{[81]} Briefly, S2 (734 mg, 1.8 mmol, 1 eq) and N,N′-Diphenylformamidine (395 mg, 2.0 mmol, 1.1 eq) were dissolved in 6 mL of Ac₂O and the mixture was refluxed at 120° C. for 30 min under argon, then cooled to room temperature. S1 (660 mg, 2.2 mmol, 1.2 eq) was suspended in 6 mL of dry pyridine and added to the reaction mixture. The reaction was stirred for 18 h at room temperature, with protection from light. The solvents were removed under reduced pressure at 60° C. The pink solid was dissolved in 20 mL DCM and washed 4× with 40 mL dH₂O, 1× with 40 mL of 1 M HCl_{(aq)}, and 1× with 40 mL of sat. NaCl. The organic layer was dried over Na₂SO₄ and concentrated under reduced pressure. The crude mixture was purified by column chromatography, eluting with a gradient of 0-10% MeOH in DCM. Cy3 was obtained as a metallic green-gold solid (479 mg, 0.99 mmol, 54% yield). ¹H NMR (Varian, 400 MHz, DMSO-d6): δ=12.04 (br. s, 1H), 8.33 (t, J=13.5 Hz, 1H), 7.62 (d, J=7.4 Hz, 2H), 7.45-7.41 (m, 4H), 7.30-7.26 (m, 2H), 6.49 (dd, J=13.5 Hz, 4.5 Hz, 2H), 4.09 (t, J=7.4 Hz, 2H), 3.64 (s, 3H), 2.20 (t, J=7.2 Hz, 2H), 1.73-1.67 (m, 14H), 1.58-1.52 (m, 2H), 1.45-1.39 (m, 2H) ppm.

### Cyanine-3 N-Hydroxysuccinimide ester (Cy3-NHS)

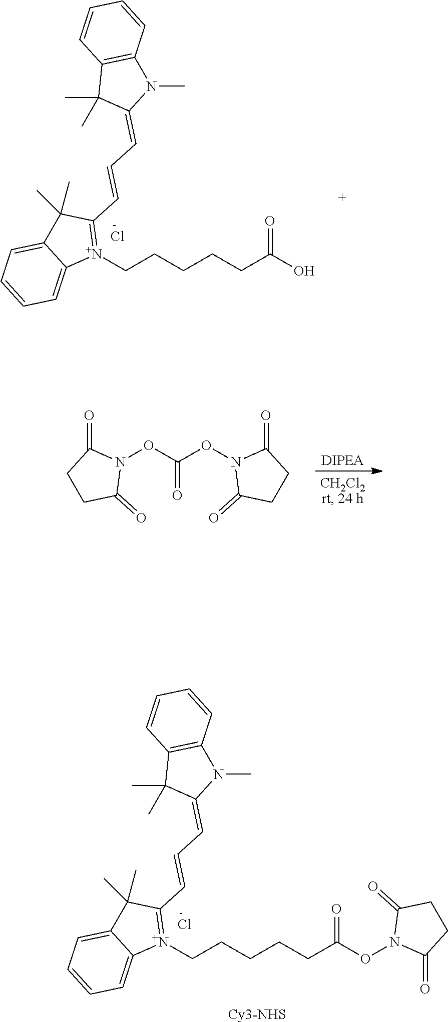

Cy3 was activated as Cy3-NHS by adapting a previously described procedure.^{[81]} Briefly, Cy3 (479 mg, 0.99 mmol, 1 eq) was dissolved in 10 mL of dry DCM. DIPEA (345 μL, 1.98 mmol, 2 eq) and DSC (567 mg, 2.18 mmol, 2.2 eq) were added and the reaction was stirred for 24 h at room temperature, with protection from light. The mixture was diluted with an additional 10 mL DCM and washed 4× with 40 mL dH₂O, 1× with 40 mL of 1 M HCl_{(aq)}, and 1× with 40 mL of sat. NaCl. The organic layer was dried over Na₂SO₄ and dried under reduced pressure to give Cy3-NHS as a metallic green-gold solid (554 mg, 0.94 mmol, 95% yield). The purity of the product was confirmed by HPLC-MS analysis.

### Purification of PEG-NH₂

Purification of PEG-NH₂ was adapted from a previously described procedure.^{[55]} Briefly, MeO-PEG-NH₂ (M_{w}=5,000 g/mol) (60 mg, 12 μmol) was dissolved in 1 mL of milli-Q H₂O and cooled to −80° C. This solution was lyophilized at 40 mT for 48 h, yielding a fluffy white solid, which was stored at −20° C. under nitrogen.

### Cyanine-3-PEG (Cy3-PEG)

Cy3-PEG was synthesized by adapting a previously described procedure.[73] Briefly, Cy3-NHS (7.1 mg, 12 μmol, 2 eq) and MeO-PEG-NH₂ (M_{w}=5,000 g/mol) (30 mg, 6 μmol, 1 eq) were dissolved in 1 mL of anhydrous DMSO, and the mixture was stirred for 18 h, with protection from light. Then, the mixture was diluted in 20 mL of milli-Q H₂O and lyophilized. The resulting residue was dissolved in 1 mL of 180 mM NaCl solution and purified on G-15 Sephadex. The resulting fractions were combined and lyophilized, giving Cy3-PEG as a pink solid (30 mg, 5.9 μmol, 92% yield). The purity of the product was confirmed by HPLC-MS analysis.

### Synthesis of Cy7.5-PEG

### Purification of PEG-NH₂

Purification of PEG-NH₂ was adapted from a previously described procedure.^{[55]} Briefly, MeO-PEG-NH₂ (M_{w}=5,000 g/mol) (60 mg, 12 μmol) was dissolved in 1 mL of milli-Q H₂O and cooled to −80° C. This solution was lyophilized at 40 mT for 48 h, yielding a fluffy white solid, which was stored at −20° C. under nitrogen.

### Cyanine-7.5-PEG (Cy7.5-PEG)

Cy7.5-PEG was synthesized by adapting a previously described procedure.^{[73]} Briefly, Cy7.5-NHS (7.0 mg, 6 μmol, 1 eq) and MeO-PEG-NH₂ (M_{w}=5,000 g/mol) (30 mg, 6 μmol, 1 eq) were dissolved in 1 mL of anhydrous DMSO, and the mixture was stirred for 18 h, with protection from light. Then, the mixture was diluted in 20 mL of milli-Q H₂O and lyophilized. The resulting residue was dissolved in 1 mL of 180 mM NaCl solution and purified on G-15 Sephadex. The resulting fractions were combined and lyophilized, giving Cy7.5-PEG as a green solid (29 mg, 5.0 μmol, 83% yield). The purity of the product was confirmed by HPLC-MS analysis.

### Reduction of the Cyanine Dye (H-Cy5, H-Cy5-PEG)

Cy5 and Cy5-PEG were converted to their reduced forms (H-Cy5 and H-Cy5-PEG) by adapting a previously described procedure.^{[82]} Unconjugated Cy5 (1 mg, 1.93 μmol, 1 eq) was dissolved in 300 μL MeOH, and 500 μL of a 1 mg·mL⁻¹ solution of NaBH₄ in MeOH (13.2 μmol, 6.8 eq) was added. Cy5-PEG (5.3 mg, 0.96 μmol, 1 eq) was dissolved in 150 μL of 1:1 MeOH/degassed H₂O, and 250 μL of a 1 mg·mL⁻¹ solution of NaBH₄ in MeOH (7.9 μmol, 8 eq) was added. The loss of blue color indicated that the reaction had occurred, yielding H-Cy5 or H-Cy5-PEG. The fluorescence intensity was measured using a Spark© multimode fluorescence microplate reader (Tecan Group, Ltd., Mannedorf, Switzerland), and the reduction was considered complete if the value obtained was <500 (λₑₓ=630 nm, λₑₘ=675 nm, gain 125).

### Re-Oxidation with Fenton's Reagent in Solution

The re-oxidation of H-Cy5 and H-Cy5-PEG was carried out by adapting a previously described procedure.^{[82]} A 10 μM solution of H-Cy5 or H-Cy5-PEG was prepared in H₂O. FeSO₄ and H₂O₂ were added, yielding final concentrations of 5 μM-100 μM and 50 μM-1 mM respectively. The fluorescence (λₑₓ=630 nm, λₑₘ=675 nm) intensity was monitored over 1 h at 23° C. in a black 96-well plate (Brand GMBH & Co., Wertheim, Germany) using a Spark© multimode fluorescence microplate reader (Tecan Group Ltd., Mannedorf, Switzerland).

### Stability of H-Cy5 and H-Cy5-PEG in Polymer Solutions

The stability of H-Cy5 or H-Cy5-PEG in common MN polymers was determined by mixing the dyes with concentrated solutions of the polymers and observing the fluorescence over time. Solutions of polymer were prepared in 5 mL dH₂O at the appropriate concentrations (250 mg·mL⁻¹ for PVP K-12, ULMW HA, and (8:1) ULMW HA/Dex; 167 mg·mL⁻¹ for PVA; and 51.4 mg·mL⁻¹ for SLMW HA). H-Cy5 or H-Cy5-PEG was added to each solution for a final concentration of 10 μM. These solutions were stored in the dark, samples were periodically taken over a 24 h period, and their fluorescence (λₑₓ=630 nm, λₑₘ=675 nm) was monitored at 23° C. in a black 96-well plate (Brand GMBH & Co., Wertheim, Germany) using a Spark® multimode fluorescence microplate reader (Tecan Group Ltd., Mannedorf, Switzerland).

### Preparation of MNs Containing Sensor and Reference Dyes H-Cy5-PEG and Cy3-PEG or Cy7.5-PEG

MNs were manufactured by adapting a previously described solvent casting method.^{[17]} Briefly, to 2 mL of dH₂O, ULMW HA (1.917 g) and dextran (0.234 g) were added and mixed thoroughly. The mixture was heated for 30 mins in a 75° C. oven and centrifuged (Sorvall ST 16R, ThermoFisher Scientific, Waltham, MA) for 5 mins at 4700 g. H-Cy5 and Cy3 (or H-Cy5-PEG and Cy3-PEG or Cy7.5-PEG) were added, resulting in concentrations of 150 μM (H-Cy5(-PEG)) and 100 μM (Cy3(-PEG) or Cy7.5(-PEG)) respectively. Using a 1 mL syringe, roughly 100 μL of this solution was cast into PDMS molds (Micropoint Technologies Pte. Ltd., Singapore) and these molds were secured with tape in 6-well cell culture plates (Sarstedt AG & Co., Nümbrecht, Germany). The plates were covered, secured with parafilm, and centrifuged for 5 mins at 2300 g. After centrifugation, polymer solution was re-applied, and the plates were rotated 180° and centrifuged again. This process was repeated a total of four times. After the final centrifugation, any excess polymer solution was removed from the molds using a spatula, and the molds were placed in a vacuum chamber at 150 mbar for 30 minutes. Roughly 100 μL of dye-free polymer solution was added to each mold to form a backing layer, and the MNs were allowed to dry for 18-24 h at 25° C. and 60% humidity, after which they were removed from the molds (FIG. 3).

### Characterization of Mechanical Properties of MNs

The failure force of the MNs was evaluated using a TA.XT-Plus Texture Analyzer (Stable Micro Systems, Surrey, UK) performing a return-to-start test. MNs were prepared as previously described, with or without addition of dye. MNs were placed on an aluminum plate and a cylindrical stainless-steel probe (6 mm diameter) was moved towards the MNs. The probe moved at 2 mm·s⁻¹, with a maximum travel distance of 300 μm (sufficient to induce MN deflection), after which the probe was released and returned to its starting position. The MN failure force was measured as the maximum of the force-displacement curve.^{[83]}

### Determination of log P of PEGylated Cyanines

The log P values of PEGylated cyanines were experimentally determined using the established shake-flask method.^{[84]} A 5 mg·mL⁻¹ solution of PEGylated cyanine dye was prepared in DMSO, and a 20 μL sample was diluted using 480 μL of water-saturated n-octanol. The samples were stirred on a Vortemp 56 (Labnet, Edison, NJ) for 5 minutes at 500 rpm, then centrifuged for 5 minutes at 2000 rpm (Mikro 120, Hettich, Tuttlingen, Germany). 500 μL of phosphate buffer (pH=6.8) was added, and samples were stirred for 72 hours, then centrifuged for 5 minutes at 2000 rpm. 50 μL of the organic phase was diluted in 450 μL of acetone and 500 μL of a 1:1 mixture of ACN and H₂O; and 50 μL of the aqueous phase was diluted in 450 μL of a 1:1 mixture of ACN and H₂O. The fluorescence intensity (λₑₓ=630 nm, λₑₘ=675 nm for Cy5-PEG; λₑₓ=530 nm, λₑₘ=575 nm for Cy3-PEG) was monitored at 23° C. in a black 96-well plate (Brand GMBH & Co., Wertheim, Germany) using a Spark® multimode fluorescence microplate reader (Tecan Group Ltd., Mannedorf, Switzerland).

### Re-Oxidation of H-Cy5-PEG/Cy3-PEG Microneedle Tattoo in Agarose Gels

A 3% w/w solution of agarose in water was prepared using a standard microwave, and 5 mL portions were dispensed into the wells of a 12-well cell culture plate (Sarstedt AG & Co., Nümbrecht, Germany). Immediately, FeSO₄ and H₂O₂ (or an equivalent volume of H₂O) were added, yielding final concentrations of 5 μM-50 μM and 50 μM-500 μM respectively. The wells were mixed thoroughly using a glass rod and allowed to cool and solidify for 2-3 h. Using a commercially available spring-loaded applicator generating an impact rate of 2 m·s⁻¹, an impact force of 1.6 N, and with a spring constant of 1 N·mm⁻¹ (values provided by the manufacturer, Micropoint Technologies Pte. Ltd., Singapore), MNs containing H-Cy5-PEG and Cy3-PEG were applied to the gels for approximately 90 seconds, until the tips had fully dissolved. The fluorescence (λₑₓ=630 nm, λₑₘ=675 nm for Cy5-PEG; λₑₓ=530 nm, λₑₘ=575 nm for Cy3-PEG) of the gels was then monitored on a Spark® multimode fluorescence microplate reader.

### Delivery of Cy5-PEG/Cy3-PEG Microneedle Tattoo to Rat Skin

A deceased rat was obtained, and the skin of its back was shaved using a manual blade razor. The skin was then treated with depilatory cream for 5 minutes to remove any remaining hair. MNs containing Cy5-PEG and Cy3-PEG were applied for 2 minutes using a spring-loaded applicator, after which the backing layer was removed from the skin. The skin sections were examined using a fluorescence stereomicroscope (AxioZoom.V16, Zeiss, Oberkochen, Germany) equipped with NIR settings (Excelitas Technologies X-Cite© Xylis light source; Photometrics® Prime™ 95B camera) using Cy5 and Cy3 filter sets (Cy5: λₑₓ=630 nm, λₑₘ=675 nm; Cy3: λₑₓ=530 nm, λₑₘ=575 nm).

### Delivery of H-Cy5-PEG/Cy7.5-PEG Microneedle Tattoo to Reconstructed Human Skin Models

The inflammatory skin disease models were generated according to previously published methods.^{[76]} Here, normal primary human keratinocytes and fibroblasts were isolated from juvenile foreskin or excised human belly skin (UBC CREB, approval number H19-00259) and cultivated according to standard protocols until skin equivalent construction. For filaggrin knockdown, keratinocytes were transfected with a mixture of HiPerFect® transfection reagent (Qiagen, Hilden, Germany) and 50 nM target siRNA or control siRNA according to the manufacturer's instructions 24 h before skin equivalent construction.

For skin equivalent construction, fibroblasts (3×10⁵/skin equivalent), fetal calf serum (0.3 mL) and bovine collagen I (PureCol, Advanced BioMatrix, San Diego, USA) were brought to a neutral pH and poured into 3D cell culture inserts with a growth area of 4.2 cm² (BD Biosciences, Heidelberg, Germany). After cultivation for 2 h at 37° C., keratinocyte growth medium was added, and the system was transferred to an incubator with 5% CO₂ and 95% humidity. After 2 h, 4.2×10⁶ primary keratinocytes (with or without filaggrin knockdown) were added on top of the collagen matrix. The skin equivalents were lifted to the air-liquid interface 24 h later and cultivated for 14 days with media changes every second day. Starting at day 10, the culture media was supplemented with 5 ng-mL¹ IL-4 and IL-13. Successful filaggrin knockdown was verified via RT-PCR. The efficiency of the filaggrin knockdown was 96±4.3% (n=3).

At day 14, the MNs containing H-Cy5-PEG and Cy7.5-PEG were inserted into the skin models using a spring-loaded applicator. The MNs rapidly dissolved within 2 minutes, after which the backing layer was carefully removed. The fluorescence intensities of the sensor and reference dye in the skin models were determined using an IVIS Lumina II imaging system (Perkin Elmer) at 0, 5, 15 and 20 mins after MN application. For Cy5, the excitation wavelength was set to 640 nm and emission wavelength 720 nm, while for Cy7.5 the excitation/emission was set to 745/840 nm, with 5 seconds exposure, binning large and f/stop 8. The fluorescence signal for each model was assessed by selecting a region of interest (ROI) and by the signal-to-noise ratio (Cy5 ROI/Cy7.5 ROI).

### Description and Validation of Portable Fluorescence Reader

The portable, battery-operated, multi-wavelength handheld fluorescence reader was custom-made for this application. The device has a circular field of view with a diameter of 3 cm. The optical and electronic components are enclosed in a custom-designed 3D-printed case made of black poly(lactic acid) (FIG. 13a). Careful optical design has been performed to ensure homogeneous illumination of the circular field as well sensitive emission measurements at different wavelengths. In short, the optical set-up consists of an exchangeable module containing an excitation source (two laser diodes (LDs) placed opposite to each other, illuminating the surface at an angle of 65°) and an emission filter set-up consisting of a 4 mm diameter pinhole, two lenses and a band-pass (BP) filter (FIG. 13a). The device used three exchangeable modules, designed for the measurement of three fluorophores: Cy3 (LD λₑₓ=520 nm; BP filter λₑₘ=555-590 nm), Cy5 (LD λₑₓ=638 nm; BP filter λₑₘ=668-702 nm), and Cy7.5 (LD λₑₓ=782 nm; BP filter λₑₘ=810-860 nm). The fluorescent light emitted by the fluorophore after laser illumination passes through the emission filter set-up and then reaches a photodiode detector that generates a photocurrent proportional to the incident light intensity. The photocurrent is acquired by the device's electronics and control algorithm and is ultimately displayed on the reader's screen as a current reading in nA (FIG. 13c).

For validation of the portable fluorescence reader, solutions of either Cy5 or Cy7.5 were prepared in ethanol at concentrations ranging from 1-100 μM. Briefly, 200 μL of a solution was placed on a glass microscope slide and covered with a glass cover slip, and the fluorescence intensity was measured with the device in a dark room. This process was repeated for each concentration of Cy5 and Cy7.5 to determine the linear dynamic range of the reader.

For validation of the portable fluorescence reader with microneedle tattoos in the skin, MNs were prepared with increasing concentrations of Cy5-PEG and Cy7.5-PEG from 50-250 μM, then applied to shaved excised porcine skin for 2 minutes using a spring-loaded applicator. Following application, MNs were removed, and fluorescence was measured at both Cy5 and Cy7.5 wavelengths. This process was repeated for each concentration of MNs.

### Reoxidation of H-Cy5-PEG/Cy7.5-PEG Microneedle Tattoo in Excised Porcine Skin

Excised porcine skin was obtained and shaved using a manual blade razor to remove any hair. The skin was permeabilized using a 1.0 mm dermaroller (DermaRollerSystem.com, Markham, ON), 200 μL of either distilled H₂O, 100 mM H₂O₂, or Fenton's Reagent (40 μM FeSO₄/400 μM H₂O₂) was added and allowed to soak into the skin for 20 minutes, after which any excess was removed with a paper towel. Following this, MNs containing H-Cy5-PEG/Cy7.5-PEG were applied for 2 minutes using a commercially available spring-loaded applicator. After removal of the MN backing layer, the fluorescence of the application site was measured with the portable fluorescence reader, using both the Cy5 and Cy7.5 filters (Cy5: λₑₓ=638 nm, λₑₘ=668-702 nm; Cy7.5: λₑₓ=782 nm, λₑₘ=810-840 nm). For each MN application, 3 measurements were recorded at each wavelength.

### Delivery of H-Cy5-PEG/Cy7.5-PEG Microneedle Tattoo In Vivo in Rats

### Animals

Male Sprague-Dawley rats (Charles River, St-Constant, QC) with weight ranging from 300-350 g at delivery were used for this study. Following arrival in the animal facility, all animals were subjected to a general health evaluation. An acclimation period of 5 days was allowed before the beginning of the study. Each animal cage was equipped with a manual water distribution system. A standard certified commercial rodent diet was provided ad libitum. Tap water was provided ad libitum at all times.

This study was approved by the Cegep Levis-Lauzon and University of Montreal Animal Care Committees and complied with CACC standards and regulations governing the use of animals for research.

### UV Irradiation and In Vivo Imaging

Rats dorsal skin was shaved and depilated under isoflurane-induced anesthesia using an electric razor and depilating cream (Nair, Church & Dwight) the day before experimentation. Rats were extensively rinsed to prevent burning with residual depilating agent. For irradiation, animals were anesthetized with isoflurane and placed under the UV irradiating lamp (Daavlin 24″ research unit×305/350-6/6) equipped with a UV power meter to manually validate constant dosimetry. Irradiated animal received 700 mJ·cm² of UVB.

After, MNs were applied under anesthesia using a commercially available spring-loaded applicator for 2 minutes for each application. Three MN arrays were applied at the same time on different sites of the dorsal skin. MNs were removed, and the application sites were scanned for fluorescence using the portable fluorescence reader. On the first day, sites were read after 0.5, 1, 4, and 6 h. On the second day, new MNs were applied and read immediately or 6 h after application. New MNs were also applied on day 3 and 4 post-irradiation.

After the last fluorescence readings, animals were sacrificed, and MNs application sites were dissected and embedded in OCT-sucrose (20%) in isopentane chilled by dry ice and then stored at −80° C. until sectioning with a cryostat.

### Statistical Analysis

Mean and standard deviation are reported for all data. Significance was analyzed using the Mann-Whitney non-parametric t-test (p-value <0.05) for comparisons between two datasets, or two-way ANOVA (p-value <0.05) for comparisons between multiple datasets as a function of time (FIGS. 10e, 12f) with Tukey's HSD post hoc test to correct for multiple comparisons. Bartlett's test was used to confirm homogeneity of variance, and the Shapiro-Wilk normality test was used to confirm normality of distribution. All graphs were plotted using GraphPad Prism 7, with error bars representing mean±standard deviation (San Diego, CA). Statistical significance is denoted using *, where (*) p s 0.05; (**) p s 0.01; (***) p s 0.001; (****) p<0.0001.

### Example 10 pH Sensing Dyes and Cation Sensing Dyes

### Synthesis of Terpyridine pH-Sensing Domain

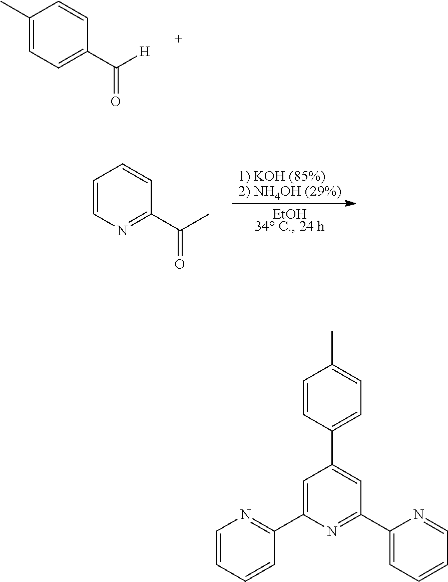

MTP was synthesized following a previously described procedure.^{[85]} Briefly, p-tolualdehyde (2.36 mL, 20 mmol, 1 eq) and 2-acetylpyridine (4.49 mL, 40 mmol, 2 eq) were dissolved in 100 mL anhydrous ethanol at 34° C. Potassium hydroxide (KOH) (3.09 g, 40 mmol, 2 eq) was added dropwise and the reaction mixture was stirred until dissolution. Concentrated aqueous ammonia (29%, 60 mL, 400 mmol, 20 eq) was then added and the solution was stirred for 24 h at 34° C. The crude product was precipitated by cooling to 0° C., and the resulting white solid was collected by vacuum filtration and washed 3× with 25 mL of ethanol (EtOH). The pure product was obtained by recrystallization in 50 mL of EtOH, yielding small colorless needle-like crystals of MTP (3.02 g, 9.3 mmol, 46.6% yield). ¹H NMR (Varian, 400 MHz, DMSO-d6): δ=8.76 ppm (d, 2H), 8.69 ppm (s, 2H), 8.66 ppm (d, 2H), 8.03 ppm (m, 2H), 7.83 ppm (d, 2H), 7.52 ppm (m, 2H), 7.40 ppm (d, 2H), 2.39 ppm (s, 3H). Identity of product was confirmed by LC-MS: m/z=323.1, [M]⁺.

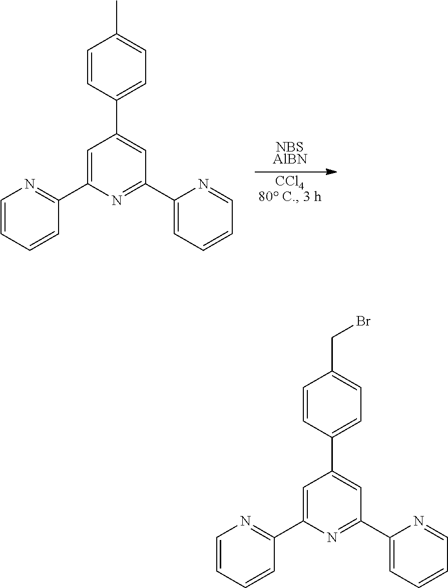 [SMILES: Cc1ccc(-c2cc(-c3ccccn3)nc(-c3ccccn3)c2)cc1 ; "NBSAlBN" ; "CCl480° C., 3 h" ; BrCc1ccc(-c2cc(-c3ccccn3)nc(-c3ccccn3)c2)cc1]

MTP-Br was synthesized following a previously described procedure.^{[85]} Briefly, MTP (1.012 g, 3.1 mmol, 1 eq), N-bromosuccinimide (NBS) (0.675 g, 3.7 mmol, 1.2 eq), and azobisisobutyronitrile (AIBN) (0.044 g, 0.24 mmol, 0.077 eq) were dissolved in 10 mL carbon tetrachloride (CCl₄). The solution was refluxed for 3 h at 80° C., connected to a drying tube to protect reagents from water. After refluxing, the warm solution was filtered through celite to remove precipitated succinimide and the solvent was removed in vacuo yielding the crude product as an off-white solid. The pure product was obtained by recrystallization in 50 mL of ethanol, yielding an off-white chalky powder of MTP-Br (0.8458 g, 2.10 mmol, 67.8% yield). ¹H NMR (Varian, 400 MHz, DMSO-d6): δ=8.78 ppm (d, 2H), 8.74 ppm (s, 2H), 8.69 ppm (d, 2H), 8.06 ppm (td, 2H), 7.96 ppm (d, 2H), 7.68 ppm (d, 2H), 7.55 ppm (m, 2H), 4.83 ppm (s, 2H). Identity of product was confirmed by LC-MS: m/z=401.1, [M]⁺.

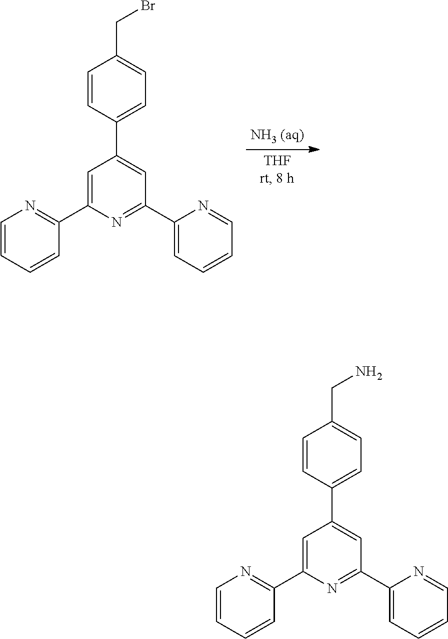 [SMILES: BrCc1ccc(-c2cc(-c3ccccn3)nc(-c3ccccn3)c2)cc1 ; NCc1ccc(-c2cc(-c3ccccn3)nc(-c3ccccn3)c2)cc1]

MTP-NH₂ was synthesized following a previously described procedure.^{[85]} Briefly, 32 mL of tetrahydrofuran (THF) and 64 mL of aqueous ammonia (29%) were combined, forming a biphasic mixture. Separately, MTP-Br (0.800 g, 2.0 mmol, 1 eq), was dissolved in 64 mL THF, and this solution was added dropwise to the THF/ammonia mixture over the course of 4 h. The mixture was allowed to stir for an additional 4 h, then transferred to a separatory funnel, where the organic layer was isolated, dried over anhydrous magnesium sulfate (MgSO₄) and filtered, after which the solvent was removed in vacuo, yielding the crude product as a yellow solid of MTP-NH₂ (0.6226 g, 1.83 mmol, 92.0% yield). Crude product was used without further purification. Identity of product was confirmed by LC-MS: m/z=338.1, [M]⁺.

### Synthesis of Cl-Cyanine-7 Fluorophore

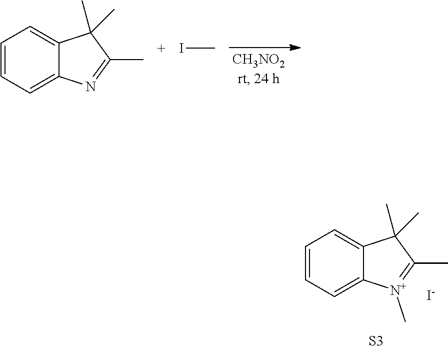

S3 was synthesized following a previously described procedure.^{[86]} Briefly, 2,3,3-trimethylindolenine (2.52 mL, 15.7 mmol, 1 eq) was diluted in 9 mL nitromethane. Iodomethane (1.95 mL, 31.4 mmol, 2 eq) was added dropwise and the reaction mixture was stirred at room temperature for 24 h under argon. The crude product was precipitated using 60 mL of diethyl ether (Et₂O), and the resulting purple solid was collected by vacuum filtration and washed 3× with 15 mL of Et₂O. The purified product was obtained as a pink powder S3 (4.17 g, 13.8 mmol, 88.0% yield). ¹H NMR (Varian, 400 MHz, DMSO-d6): δ=7.90 ppm (m, 1H), 7.81 ppm (m, 1H), 7.60 ppm (m, 2H), 3.96 ppm (s, 3H), 2.75 ppm (s, 3H), 1.51 ppm (s, 6H). Identity of product was confirmed by LC-MS: m/z=174.1, [M]⁺.

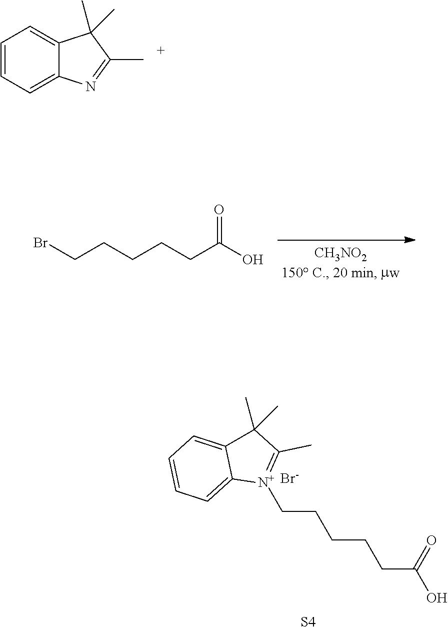 [SMILES: CC1=Nc2ccccc2C1(C)C ; O=C(O)CCCCCBr ; CC1=[N+](CCCCCC(=O)O)c2ccccc2C1(C)C ; [Br-] ; "S4"]

S4 was synthesized by adapting a previously described procedure.^{[86]} Briefly, 2,3,3-trimethylindolenine (0.642 mL, 4.0 mmol, 1 eq) and 6-bromohexanoic acid (0.936 g, 4.8 mmol, 1.2 eq) were dissolved in 0.8 mL nitromethane in a sealed tube under argon. The reaction mixture was heated under microwave irradiation at 120° C. for 20 minutes. The crude product was dissolved in a minimal volume of methanol and precipitated using 60 mL of Et₂O and cooling to −20° C. overnight. The resulting pink solid was collected by vacuum filtration and washed 3× with 15 mL of Et₂O. The purified product was obtained as a pink powder S4 (0.918 g, 2.6 mmol, 64.8% yield). ¹H NMR (Varian, 400 MHz, DMSO-d6): δ=12.00 ppm (br. s, 1H), 7.97 ppm (m, 1H), 7.83 ppm (m, 1H), 7.60 ppm (m, 2H), 4.44 ppm (t, J=7.8 Hz, 2H), 2.84 ppm (s, 3H), 2.21 ppm (t, J=7.2 Hz, 2H), 1.83 ppm (m, 2H), 1.54 ppm (m, 8H), 1.42 ppm (m, 2H). Identity of product was confirmed by LC-MS: m/z=274.1, [M]⁺.

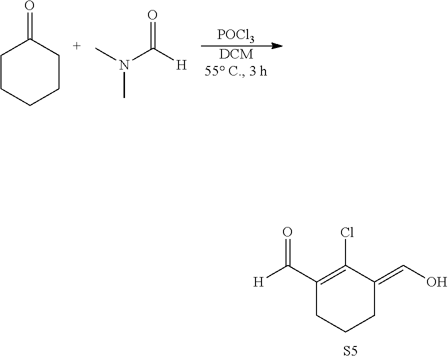

S5 was synthesized by adapting a previously described procedure.^{[87]} Briefly, anhydrous N,N-dimethylformamide (DMF) (10 mL, 129 mmol, 5 eq) was mixed with 10 mL of anhydrous dichloromethane (DCM) and cooled to 0° C. in an ice bath. Separately, phosphorus oxychloride (9.25 mL, 100 mmol, 4 eq) was mixed with 8.75 mL of anhydrous DCM, and this solution was added dropwise to the cooled DMF/DCM solution. Following this, cyclohexanone (2.64 mL, 25 mmol, 1 eq) was added, and the mixture was refluxed at 55° C. for 3 h under argon. The reaction was quenched by decanting onto 100 g of ice, and the mixture was diluted to 400 mL with distilled water. The crude product precipitated from this mixture as a yellow solid S5 (3.107 g, 18.0 mmol, 72.3%). ¹H NMR (Varian, 400 MHz, DMSO-d6): δ=10.82 ppm (s, 1H), 10.00 ppm (br. S, 1H), 7.59 ppm (br. S, 1H), 2.33 ppm (t, 4H), 1.56 ppm (quin, 2H). Identity of product was confirmed by LC-MS: m/z=172.0, [M]⁺.

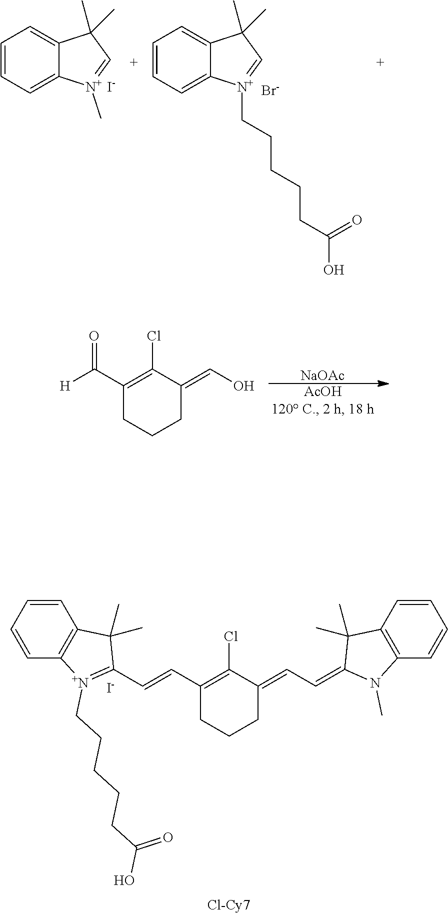

Cl-Cy7 was synthesized by adapting previously described procedures.^{[88,89]} Briefly, S4 (0.213 g, 0.6 mmol, 1.2 eq), S5 (0.087 g, 0.5 mmol, 1 eq), and sodium acetate (0.240 g, 3 mmol, 6 eq) were dissolved in 20 mL of glacial acetic acid. The mixture was refluxed at 120° C. for 2 h, then returned to room temperature, after which S3 (0.151 g, 0.5 mmol, 1 eq) was added and the mixture was returned to reflux at 120° C. for an additional 18 h. Solvent was removed in vacuo and the residue was dissolved in 20 mL of DCM and washed 3× with 20 mL portion of distilled water, followed by washing once with 20 mL of 1 M HCl_{(aq)}. The organic layer was separated and dried over anhydrous sodium sulfate (Na₂SO₄), then filtered and the solvent was removed in vacuo to give the crude product, which was purified by column chromatography on silica gel using a gradient of 0-10% methanol in DCM. The pure product was obtained as a metallic red solid of Cl-Cy7 (0.157 g, 0.22 mmol, 44.1%). ¹H NMR (Varian, 400 MHz, DMSO-d6): δ=8.25 ppm (m, 2H), 7.61 ppm (t, 2H), 7.42 ppm (m, 4H), 7.28 ppm (m, 2H), 6.29 ppm (m, 2H), 3.95 ppm (t, 2H), 3.69 ppm (s, 3H), 2.69 ppm (t, 4H), 2.27 ppm (t, 2H), 1.85 ppm (m, 2H), 1.71 ppm (m, 2H), 1.65 ppm (s, 12H), 1.57 ppm (m, 2H), 1.36 ppm (m, 2H). Identity of product was confirmed by LC-MS: m/z=583.3, [M]⁺.

### Synthesis of Crown Ether Na⁺ Sensing Domain

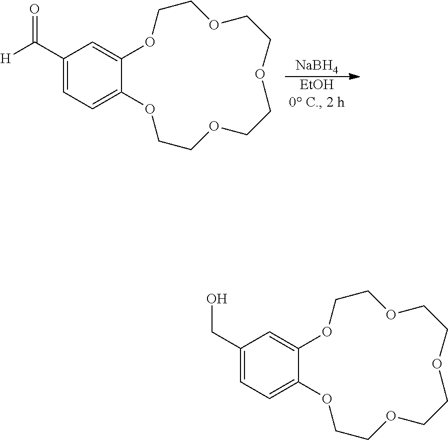 [SMILES: O=Cc1ccc2c(c1)OCCOCCOCCOCCO2 ; "EtOH0° C., 2 h" ; "NaBH4" ; OCc1ccc2c(c1)OCCOCCOCCOCCO2]

(4′)-hydroxymethylbenzo-[15-crown-5] was synthesized by adapting a previously described procedure.^{[90]} Briefly, (4′)-formylbenzo-[15-crown-5] (0.100 g, 0.34 mmol, 1 eq) was dissolved in 1 mL of ethanol and cooled to 0° C. in an ice bath, after which sodium borohydride (0.013 g, 0.34 mmol, 1 eq) was added in small portions over −15 mins, keeping the reaction temperature <7° C. The reaction mixture was stirred at 0° C. for 90 mins, after which solvent was removed in vacuo, and the crude product was dissolved in DCM and washed 3× with saturated NaCl_{(aq)}. The organic layer was separated, dried over Na₂SO₄, filtered, and the solvent was removed in vacuo yielding the pure product as a clear colorless oil of (4′)-hydroxymethylbenzo-[15-crown-5] (0.071 g, 0.24 mmol, 70.7%). ¹H NMR (Varian, 400 MHz, DMSO-d6): δ=6.90 ppm (d, 2H), 6.82 ppm (d, 1H), 4.40 ppm (d, 2H), 4.03 ppm (quar, 4H), 3.77 ppm (quar, 4H), 3.62 ppm (s, 8H), 1.02 ppm (br. s, 1H). Identity of product was confirmed by LC-MS: m/z=321.3, [M+Na]⁺.

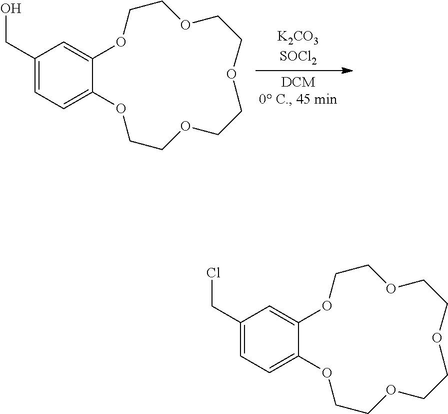 [SMILES: OCc1ccc2c(c1)OCCOCCOCCOCCO2 ; ClCc1ccc2c(c1)OCCOCCOCCOCCO2]

(4′)-chloromethylbenzo-[15-crown-5] was synthesized by adapting a previously described protocol.^{[90]} Briefly, (4′)-hydroxymethylbenzo-[15-crown-5] (0.070 g, 0.24 mmol, 1 eq) was dissolved in 5 mL of anhydrous DCM and cooled to 0° C. in an ice bath, after which potassium carbonate (K₂CO₃) (0.095 g, 0.69 mmol, 2.9 eq) was added. The reaction mixture was placed under argon flow and thionyl chloride (SOCl₂) (32.3 μL, 0.45 mmol, 1.9 eq) was added. The reaction mixture was stirred at 0° C. for 45 mins, after which it was filtered to remove K₂CO₃, and solvent was removed in vacuo yielding the crude product as a yellow amorphous solid of (4′)-chloromethylbenzo-[15-crown-5] (0.074 g, 0.24 mmol, quantitative). The crude product was used without further purification. Identity of product was confirmed by LC-MS: m/z=339.8, [M+Na]⁺.

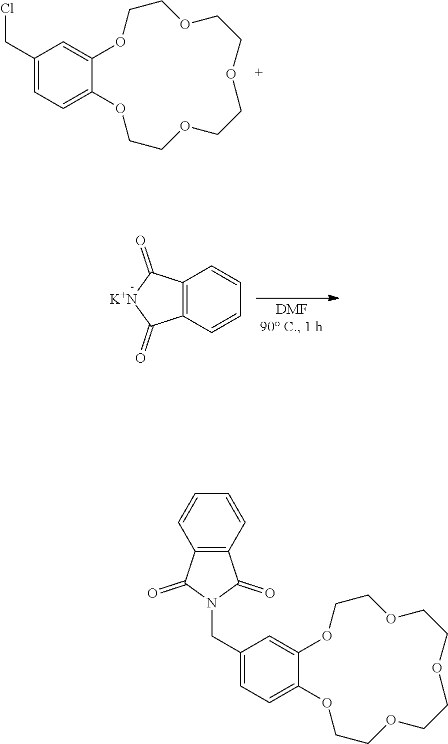

(4′)-phthalimidomethylbenzo-[15-crown-5] was synthesized by adapting a previously described protocol.^{[90]} Briefly, (4′)-chloromethylbenzo-[15-crown-5] (0.074 g, 0.24 mmol, 1 eq) and potassium phthalimide (0.048 g, 0.26 mmol, 1.1 eq) were dissolved in 2 mL of anhydrous N,N-dimethylformamide (DMF), heated to 90° C., and allowed to stir for 1 h. The reaction mixture was cooled to room temperature and diluted with 10 mL of chloroform (CHCl₃) and 20 mL of distilled water. The organic layer was separated, and the aqueous layer was further extracted with 2×20 mL portions of CHCl₃. Combined organic layers were pooled and washed 2× with 40 mL portions of 0.15 M NaOH_{(aq)}, then 4× with 40 mL portions of distilled water. After washing, the organic layer was dried over anhydrous Na₂SO₄, filtered, and the solvent was removed in vacuo yielding the pure product as a yellow solid of (4′)-phthalimidomethylbenzo-[15-crown-5] (0.057 g, 0.13 mmol, 57.1%). Identity of product was confirmed by LC-MS: m/z=450.5, [M+Na]⁺.

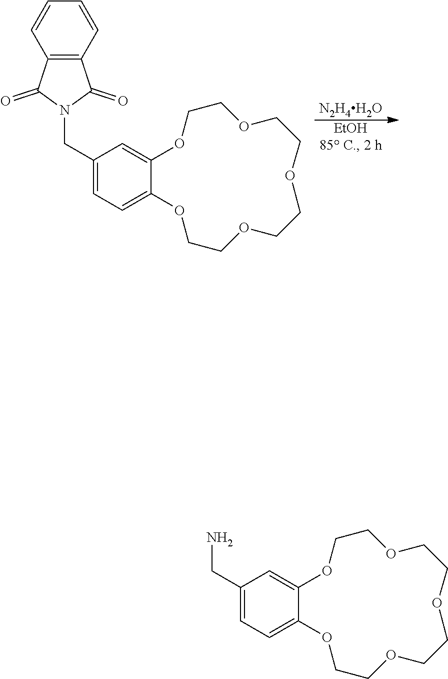 [SMILES: O=C1c2ccccc2C(=O)N1Cc1ccc2c(c1)OCCOCCOCCOCCO2 ; "N2H4·H2O" ; "EtOH85° C., 2 h" ; NCc1ccc2c(c1)OCCOCCOCCOCCO2]

(4′)-aminomethylbenzo-[15-crown-5] was synthesized by adapting a previously described protocol.^{[90]} Briefly, (4′)-phthalimidomethylbenzo-[15-crown-5] (0.057 g, 0.13 mmol, 1 eq) was dissolved in 4 mL of warm anhydrous ethanol, and hydrazine hydrate (26 μL, 0.54 mmol, 4 eq) was added. The reaction mixture was refluxed at 85° C. for 2 h, then cooled to 0° C. in an ice bath and filtered to remove precipitated phthalhydrazide, which was washed with ice-cold ethanol. Solvent was removed in vacuo, 10 mL of CHCl₃ was added to the crude product, and the mixture was cooled to −20° C. overnight in the freezer. This mixture was filtered, discarding any precipitate, and solvent was removed in vacuo. The resulting crude product was dissolved in hot acetonitrile and filtered again, and solvent was removed in vacuo yielding the pure product as a white solid of (4′)-aminomethylbenzo-[15-crown-5](0.032 g, 0.11 mmol, 78.9%). Identity of product was confirmed by LC-MS: m/z=320.3, [M+Na]⁺.

### Synthesis of Cyanine-based pH and Na⁺ Sensors

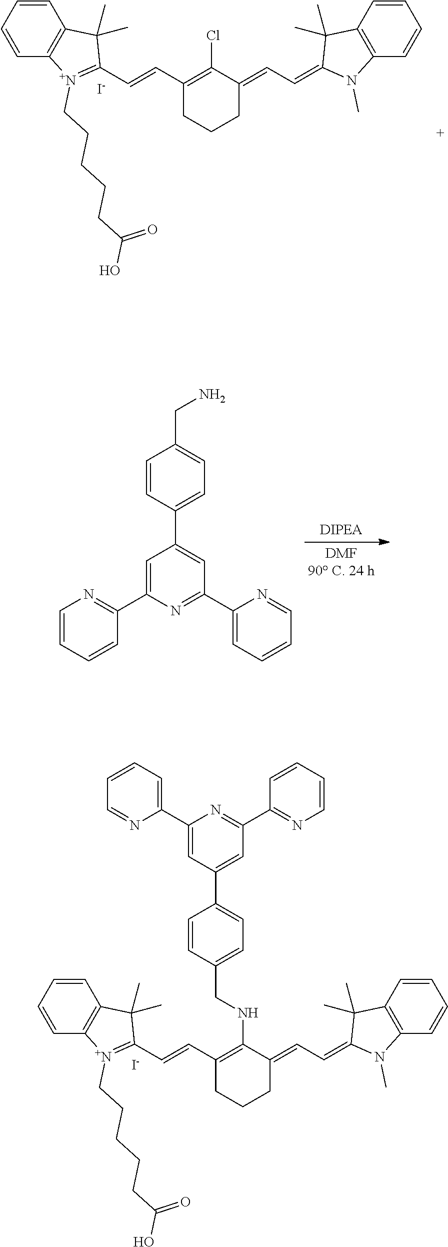 [SMILES: CN1/C(=C/C=C2\CCCC(/C=C/C3=[N+](CCCCCC(=O)O)c4ccccc4C3(C)C)=C2Cl)C(C)(C)c2ccccc21 ; [I-] ; NCc1ccc(-c2cc(-c3ccccn3)nc(-c3ccccn3)c2)cc1 ; CN1/C(=C/C=C2\CCCC(/C=C/C3=[N+](CCCCCC(=O)O)c4ccccc4C3(C)C)=C2NCc2ccc(-c3cc(-c4ccccn4)nc(-c4ccccn4)c3)cc2)C(C)(C)c2ccccc21 ; [I-]]

MTP-Cy7-COOH was synthesized by adapting a previously described protocol.^{[1]} Briefly, MTP-NH₂ (0.102 g, 0.30 mmol, 1 eq) was dissolved in 2 mL of anhydrous DMF, and diisopropylethylamine (DIPEA) (104 μL, 0.60 mmol, 2 eq) was added dropwise. Cl-Cy7-COOH (0.213 g, 0.30 mmol, 1 eq) was added and the reaction mixture was heated to 90° C. for 24 h under argon. The reaction mixture was decanted into 50 mL of distilled water to precipitate crude product, which was collected by vacuum filtration, dissolved in DCM, and washed with 2×20 mL portions of 1 M HCl_{(aq)}, followed by 1×20 mL portion of distilled water. The organic layer was separated, dried over anhydrous Na₂SO₄, and solvent was removed in vacuo. The residue was purified by column chromatography on silica gel using a gradient of 5-10% methanol in DCM. The pure product was obtained as a blue-green solid of MTP-Cy7-COOH (0.118 g, 0.12 mmol, 38.8%). Identity of product was confirmed by LC-MS: m/z=885.5, [M]⁺.

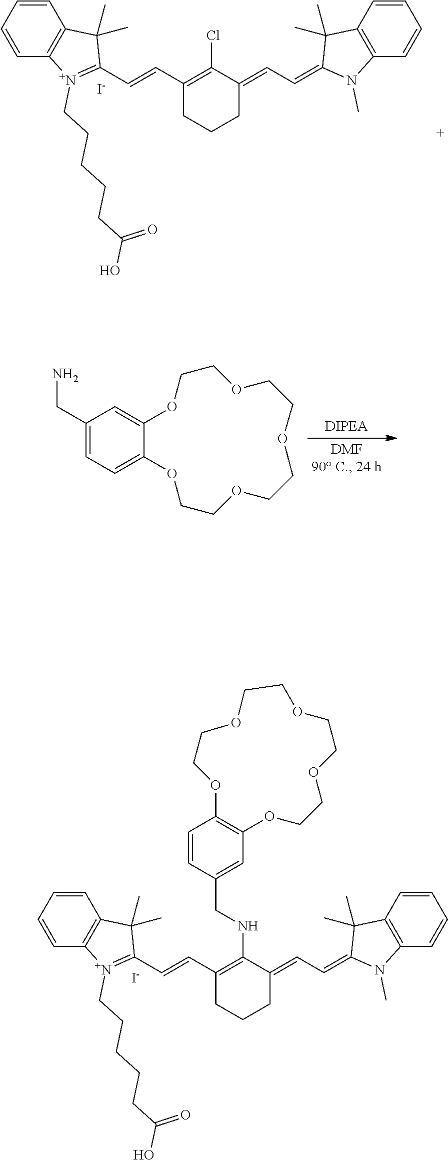 [SMILES: CN1/C(=C/C=C2\CCCC(/C=C/C3=[N+](CCCCCC(=O)O)c4ccccc4C3(C)C)=C2Cl)C(C)(C)c2ccccc21 ; [I-] ; NCc1ccc2c(c1)OCCOCCOCCOCCO2 ; CN1/C(=C/C=C2\CCCC(/C=C/C3=[N+](CCCCCC(=O)O)c4ccccc4C3(C)C)=C2NCc2ccc3c(c2)OCCOCCOCCOCCO3)C(C)(C)c2ccccc21 ; [I-]]

Benzo-15-crown-5-Cy7-COOH was synthesized using a protocol analogous to that used for MTP-Cy7-COOH. Briefly, (4′)-aminobenzyl-[15-crown-5] (0.032 g, 0.11 mmol, 1 eq) was dissolved in 1 mL of anhydrous DMF, and diisopropylethylamine (DIPEA) (37 μL, 0.21 mmol, 2 eq) was added dropwise. Cl-Cy7-COOH (0.075 g, 0.11 mmol, 1 eq) was added and the reaction mixture was heated to 90° C. for 24 h under argon. The reaction mixture was decanted into 50 mL of distilled water to precipitate crude product, which was collected by vacuum filtration, dissolved in DCM, and washed with 2×20 mL portions of 1 M HCl_{(aq)}, followed by 1×20 mL portion of distilled water. The organic layer was separated, dried over anhydrous Na₂SO₄, and solvent was removed in vacuo. The residue was purified by column chromatography on silica gel using a gradient of 5-10% methanol in DCM. The pure product was obtained as a dark blue solid of Benzo-15-crown-5-Cy7-COOH (0.023 g, 0.02 mmol, 24.9%). Identity of product was confirmed by LC-MS: m/z=844.5, [M]⁺.

### Example 11 Manufacture of Ratiometric Microneedles (MNs)

MNs were manufactured by adapting a previously described solvent casting method.^{[17]} Briefly, to 2 mL of dH₂O, ULMW HA (1.917 g) and dextran (0.234 g) were added and mixed thoroughly. The mixture was heated for 30 mins in a 75° C. oven and centrifuged (Sorvall ST 16R, ThermoFisher Scientific, Waltham, MA) for 5 mins at 4700 g. MTP-Cy7-COOH (or Benzo-15-crown-5-Cy7-COOH) and Cy5-COOH were added, resulting in concentrations of 150 μM (sensor) and 100 μM (Cy5-COOH) respectively. Using a 1 mL syringe, roughly 100 μL of this solution was cast into PDMS molds (Micropoint Technologies Pte. Ltd., Singapore) and these molds were secured with tape in 6-well cell culture plates (Sarstedt AG & Co., Numbrecht, Germany). The plates were covered, secured with parafilm, and centrifuged for 5 mins at 2300 g. After centrifugation, polymer solution was re-applied, and the plates were rotated 180° and centrifuged again. This process was repeated a total of four times. After the final centrifugation, any excess polymer solution was removed from the molds using a spatula, and the molds were placed in a vacuum chamber at 150 mbar for 30 minutes. Roughly 100 μL of dye-free polymer solution was added to each mold to form a backing layer, and the MNs were allowed to dry for 18-24 h at 25° C. and 60% humidity, after which they were removed from the molds.

### Example 12 Measurement of pH and [Na⁺] in Skin-Simulating Gels

A 3% w/w solution of agarose in water was prepared using a standard microwave, and 5 mL portions were dispensed into the wells of a 12-well cell culture plate (Sarstedt AG & Co., Numbrecht, Germany). Immediately, the pH, or concentration of NaCl, was adjusted through addition of stock solutions of HCl, NaOH, or NaCl, yielding final pH values of 5-9 or final NaCl concentrations of 0-100 mM. The wells were mixed thoroughly using a glass rod and allowed to cool and solidify for 2-3 h. Using a commercially available spring-loaded applicator generating an impact rate of 2 m-s-1, an impact force of 1.6 N, and with a spring constant of 1 N·mm⁻¹ (values provided by the manufacturer, Micropoint Technologies Pte. Ltd., Singapore), microneedles containing MTP-Cy7-COOH (or Benzo-15-crown-5-Cy7-COOH) and Cy5-COOH were applied to the gels for approximately 90 seconds, until the tips had fully dissolved. The fluorescence (λₑₓ=630 nm, λₑₘ=675 nm for Cy5-COOH; λₑₓ=760 nm, λₑₘ=805 nm for Cy7-COOH) of the gels was then monitored on a Spark© multimode fluorescence microplate reader.

### Example 13 Functionalisation and Testing of Other Polymer Cyanine Conjugates

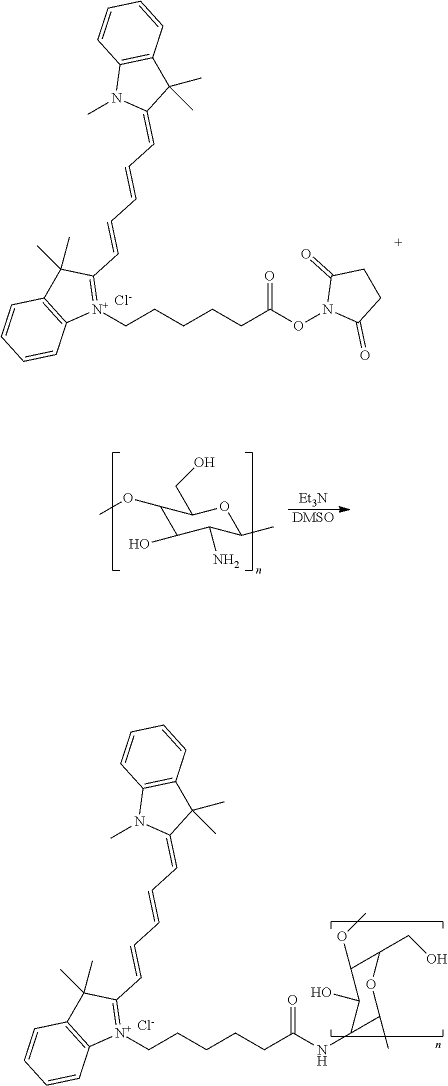

Cy5-trimethylchitosan (TMC) was synthesized using a protocol analogous to that used for Cy5-PEG.^{[73]} Briefly, a solution of Cy5-NHS (3.1 mg, 5.0 μmol, 10 eq) was prepared in 200 μL of dimethyl sulfoxide (DMSO) and added to a solution of TMC (M_{w}=60 kDa) (30 mg, 0.5 μmol, 1 eq) in 1 mL of distilled water, along with a catalytic amount of triethylamine (Et₃N). The reaction mixture was stirred at room temperature for 24 h, then diluted in 15 mL of milli-Q water, frozen, and lyophilized. The resulting residue was dissolved in 1 mL of 180 mM NaCl solution and purified on G-15 Sephadex. The resulting fractions were combined and lyophilized, giving Cy5-TMC as a light blue solid (9.1 mg, 0.15 μmol, 30.3% yield). The purity of the product was confirmed by HPLC-MS analysis. A significant loss of yield was observed due to aggregation of the Cy5-TMC into insoluble particles, visibly retained at the head of the Sephadex column, suggesting poor stability of this dye-conjugate.

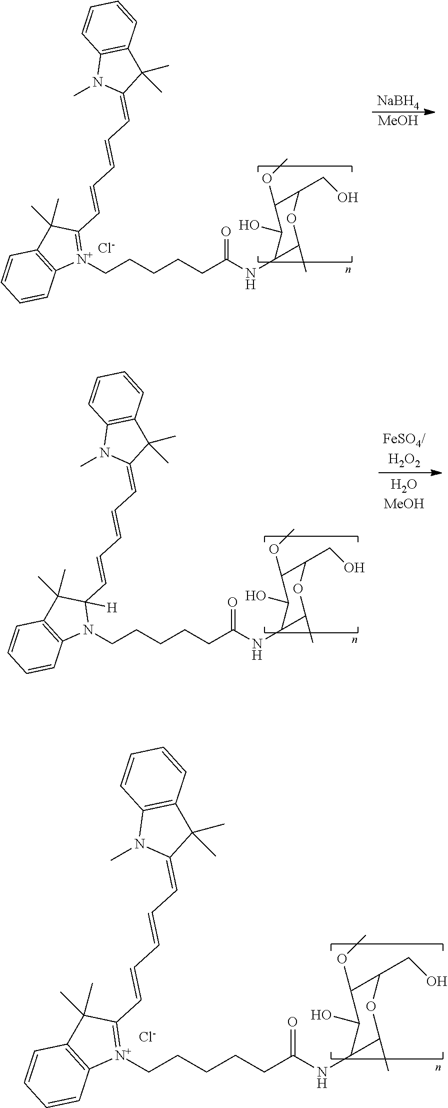

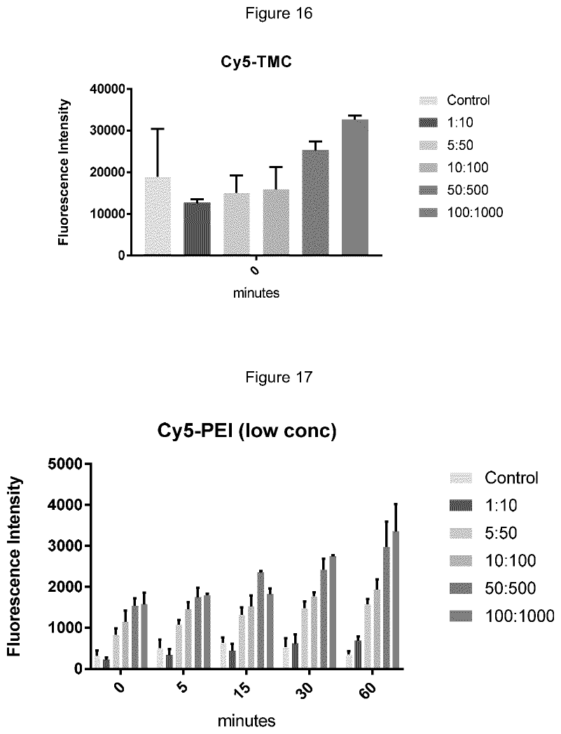

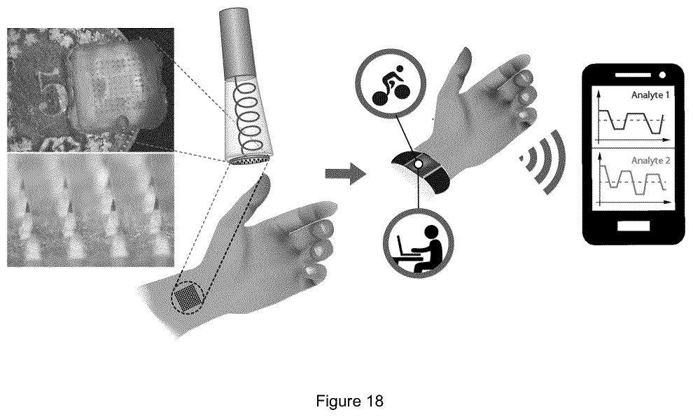

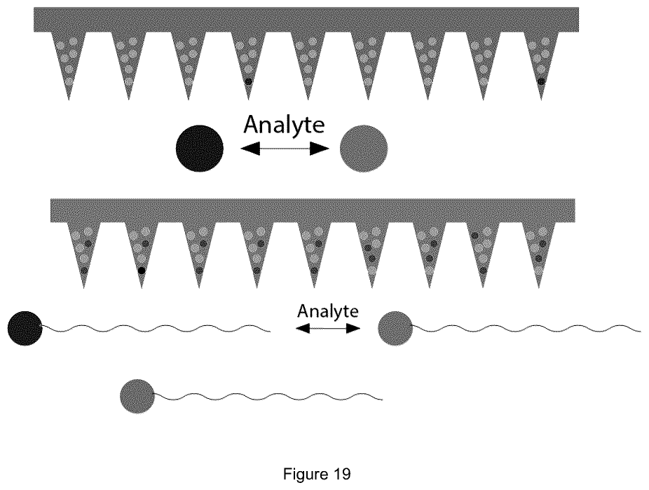

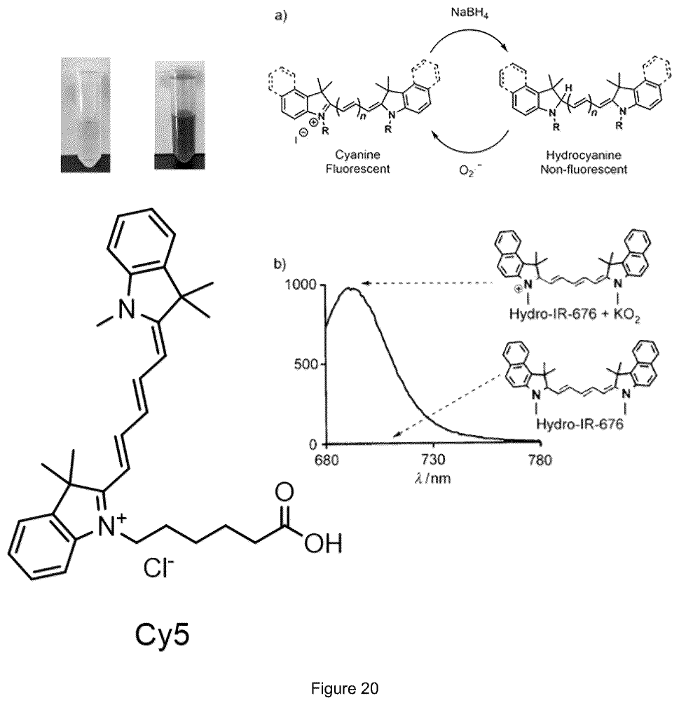

Cy5-TMC (6.0 mg, 0.1 μmol, 1 eq) was dissolved in 500 μL of distilled water, and 250 μL of a 1 mg·mL⁻¹ solution of NaBH₄ in MeOH (8 eq) was added. The loss of blue color indicated that the reaction had occurred, yielding H-Cy5-TMC. The fluorescence intensity was measured using a Spark® multimode fluorescence microplate reader (Tecan Group, Ltd., Mannedorf, Switzerland). The re-oxidation of H-Cy5-TMC was carried out by adapting a previously described procedure.^{[82]} A 10 μM solution of H-Cy5-TMC was prepared in H₂O. FeSO₄ and H₂O₂ were added, yielding final concentrations of 1 μM-100 μM and 10 μM-1 mM respectively. The fluorescence (λₑₓ=630 nm, λₑₘ=675 nm) intensity was monitored at 23° C. in a black 96-well plate (Brand GMBH & Co., Wertheim, Germany) using a Spark® multimode fluorescence microplate reader. Fluorescence intensity is shown in FIG. 16.

In this instance, incomplete reduction was observed, and a minimal increase between the control and maximum reactive oxygen species concentration was observed. This was theorized to be due to interactions between the NaBH₄ and the TMC polymer, preventing the proper reduction of Cy5 fluorescence. Between the poor stability to aggregation and incomplete reduction of dye fluorescence, this choice of polymer was not pursued further.

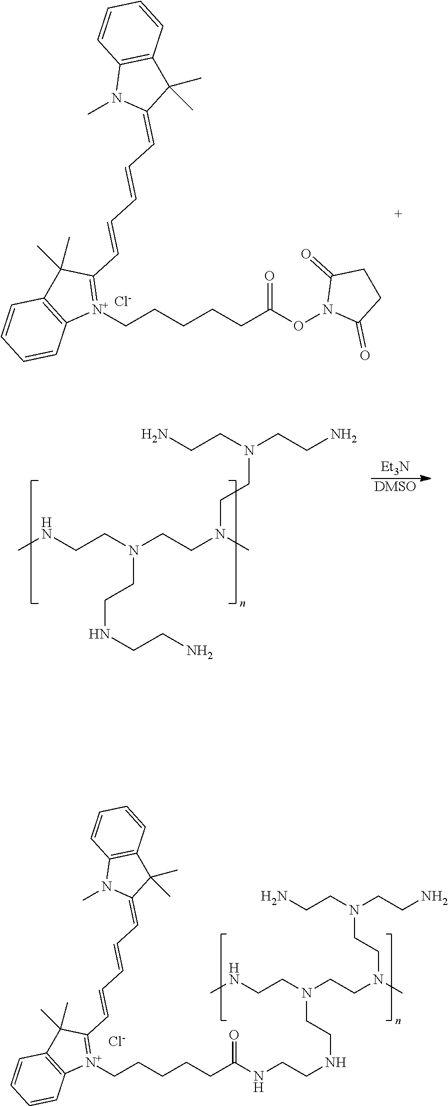

Cy5-poly(ethyleneimine) (PEI) was synthesized using a protocol analogous to that used for Cy5-PEG.^{[73]} Briefly, a solution of Cy5-NHS (4.6 mg, 7.5 μmol, 10 eq) was prepared in 200 μL of dimethyl sulfoxide (DMSO) and added to a solution of PEI (M_{w}=10 kDa) (7.5 mg, 0.75 μmol, 1 eq) in 500 μL of distilled water along with a catalytic amount of triethylamine (Et₃N). The reaction mixture was stirred at room temperature for 24 h, then diluted in 15 mL of milli-Q water, frozen, and lyophilized. The resulting residue was analyzed by HPLC-MS, displaying no trace of free Cy5, and was used without further purification. A notable color change was observed upon conjugation, where Cy5 changed from a dark blue color to a teal-green color upon conjugation to the PEI. This suggested that conjugation to this polymer had an effect on either the spectral properties or the stability of the dye.

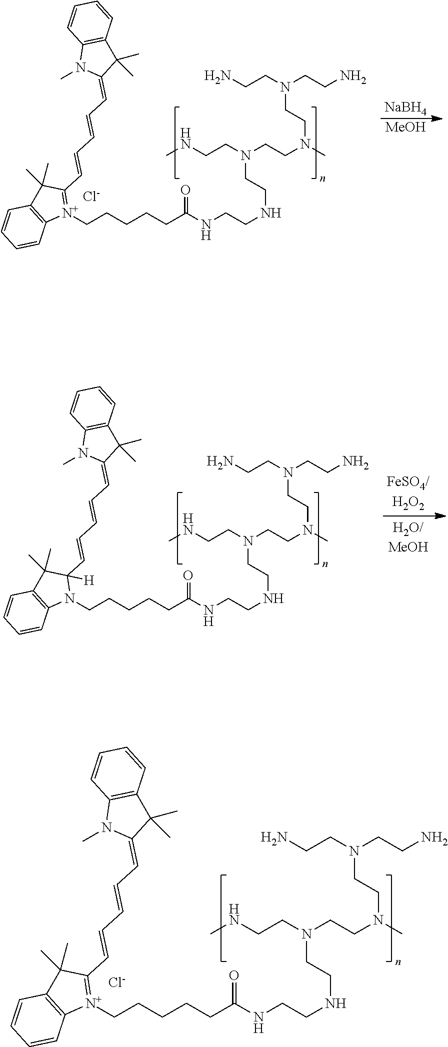

Cy5-PEI (6.0 mg, 0.1 μmol, 1 eq) was dissolved in 500 μL of distilled water, and 250 μL of a 1 mg·mL⁻¹ solution of NaBH₄ in MeOH (8 eq) was added. The loss of blue color indicated that the reaction had occurred, yielding H-Cy5-PEI. The fluorescence intensity was measured using a Spark© multimode fluorescence microplate reader (Tecan Group, Ltd., Mannedorf, Switzerland). The re-oxidation of H-Cy5-PEI was carried out by adapting a previously described procedure.^{[82]} A 10 μM solution of H-Cy5-TMC was prepared in H₂O. FeSO₄ and H₂O₂ were added, yielding final concentrations of 1 μM-100 μM and 10 μM-1 mM respectively. The fluorescence (λₑₓ=630 nm, λₑₘ=675 nm) intensity was monitored at 23° C. in a black 96-well plate (Brand GMBH & Co., Wertheim, Germany) using a Spark® multimode fluorescence microplate reader. Fluorescence intensity is shown in FIG. 17.

In this instance, reduction proceeded smoothly, however the maximum reoxidation observed was significantly reduced relative to free Cy5. This was theorized to be due to spectral effects of the PEI when conjugated to Cy5, or some stability issue reducing the maximum fluorescence intensity of the dye-conjugate, as evidenced by the color change upon conjugation. Accordingly, this choice of polymer was not pursued further.

### Example 14 Synthesis of Cy5.5 and Precursors

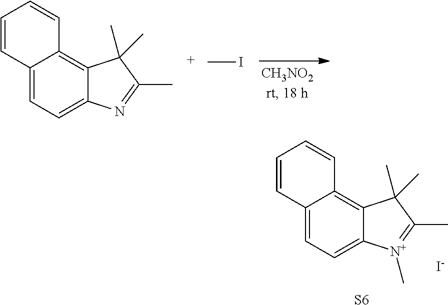

S6 was synthesized using a previously described procedure.^{[1]} Briefly, 1,1,2-trimethylbenz[e]indole (3.920 g, 15.7 mmol, 1 eq) was dissolved in 9 mL CH₃NO₂. Mel (1.95 mL, 31.4 mmol, 2 eq) was added dropwise and the reaction mixture was stirred at room temperature for 18 h under argon. The crude product was precipitated using 60 mL of Et₂O, and the resulting purple solid was collected by vacuum filtration and washed 3× with 15 mL of Et₂O. A light green solid was obtained (5.22 g, 14.9 mmol, 94.6% yield). ¹H NMR (Varian, 400 MHz, DMSO-d6), 5=8.34 ppm (d, J=8.6 Hz, 1H), 8.27 ppm (d, J=9.0 Hz, 1H), 8.19 ppm (d, 7.8 Hz, 1H), 8.08 ppm (d, 9.0 Hz, 1H), 7.76 ppm (m, 1H), 7.70 ppm (m, 1H), 4.07 ppm (s, 3H), 2.85 ppm (s, 3H), 1.73 ppm (s, 6H). Identity of product was confirmed by LC-MS: m/z=224.3, [M]⁺.

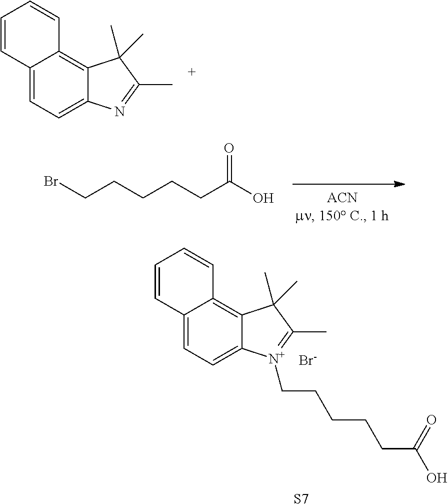 [SMILES: CC1=Nc2ccc3ccccc3c2C1(C)C ; O=C(O)CCCCCBr ; CC1=[N+](CCCCCC(=O)O)c2ccc3ccccc3c2C1(C)C ; [Br-] ; "S7"]

S7 was synthesized by adapting a previously described procedure.^{[2]} Briefly, 1,1,2-trimethylbenz[e]indole (1.925 g, 9.2 mmol, 1 eq) and 6-bromohexanoic acid (2.653 g, 13.6 mmol, 1.5 eq) were dissolved in 12 mL acetonitrile in a sealed tube under argon. The reaction mixture was heated under microwave irradiation at 150° C. for 1 hour. The crude product was precipitated by cooling to −20° C. overnight. The resulting grey solid was collected by vacuum filtration and washed 3× with 15 mL of Et₂O. The purified product was obtained as a grey powder S7 (2.233 g, 5.5 mmol, 60.0% yield). ¹H NMR (Varian, 400 MHz, DMSO-d6): δ=11.98 ppm (br. S, 1H), 8.35 ppm (d, J=8.2 Hz, 1H), 8.27 ppm (d, J=9.0 Hz, 1H), 8.20 ppm (d, 7.4 Hz, 1H), 8.13 ppm (d, 9.0 Hz, 1H), 7.77 ppm (m, 1H), 7.70 ppm (m, 1H), 4.55 ppm (t, J=7.6 Hz, 2H), 2.92 ppm (s, 3H), 2.21 ppm (t, J=7.2 Hz, 2H), 1.88 ppm (m, 2H), 1.74 ppm (s, 6H), 1.55 ppm (m, 2H), 1.45 ppm (m, 2H). Identity of product was confirmed by LC-MS: m/z=324.2, [M]⁺.

These two precursors below can be used to synthesize Cy3.5 and Cy5.5 using a procedure analogous to that used for Cy3 and Cy5.

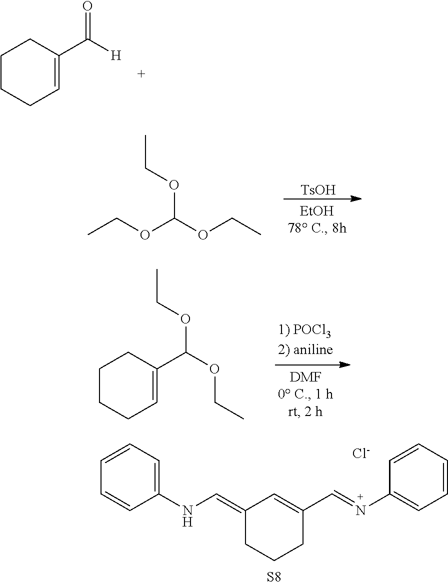

S8 was synthesized by adapting two previously described procedures.^{[87,88]} Briefly, 1-cyclohexene-1-carboxaldehyde (2.754 g, 25 mmol, 1 eq) and triethyl orthoformate (7.410 g, 50 mmol, 2 eq) were dissolved in 30 mL of anhydrous ethanol, along with a catalytic amount of p-toluenesulfonic acid (TsOH) (0.215 g, 1.25 mmol, 0.05 eq). The mixture was refluxed at 78° C. for 6 h under argon, then cooled to room temperature and neutralized to pH 7 by addition of sodium bicarbonate (NaHCO₃). The reaction mixture was filtered, solvent was removed in vacuo, and the intermediate (1-formyl-1-cyclohexene diethyl acetal) was used directly for the next step without further purification. For this step, anhydrous N,N-dimethylformamide (DMF) (10 mL, 129 mmol, 5 eq) was mixed with 10 mL of anhydrous dichloromethane (DCM) and cooled to 0° C. in an ice bath. Separately, phosphorus oxychloride (9.25 mL, 100 mmol, 4 eq) was mixed with 8.75 mL of anhydrous DCM, and this solution was added dropwise to the cooled DMF/DCM solution. Following this, the intermediate 1-formyl-1-cyclohexene diethyl acetal, in 5 mL of DCM was added, and the mixture was stirred at room temperature for 3 h under argon. The reaction mixture was then cooled to 0° C., aniline (5 mL, 55 mmol, 2.2 eq) dissolved in 5 mL of ethanol was added dropwise, and the mixture was stirred at room temperature for 1 h. The reaction was quenched by decanting onto 100 g of ice, and pH was adjusted to ˜10 using a concentrated sodium hydroxide solution. This solution was extracted using 3×30 mL of diethyl ether, acidified to pH ˜2 with concentrated hydrochloric acid, and the product was precipitated by cooling to −20° C. The crude was recrystallized from ethanol, yielding S8 (3.120 g, 9.9 mmol, 39.4%) as a purple solid. ¹H NMR (Varian, 400 MHz, DMSO-d6): δ=11.34 (br. S, 1H), 8.33 (s, 2H), 7.60 (d, 4H, J_{a,b}=7.8 Hz), 7.45 (t, 4H, J_{a,b}=J_{b,c}=7.8 Hz), 7.26 (m, 2H), 2.74 (t, 4H, J_{a,b}=J_{b,c}=5.9 Hz), 1.84 (m, 2H). Identity of product was confirmed by LC-MS: m/z=289.2, [M]⁺.

This middle linker was used for preparation of Cy7 and Cy7.5 using a methodology analogous to that provided earlier for Cy7 synthesis.

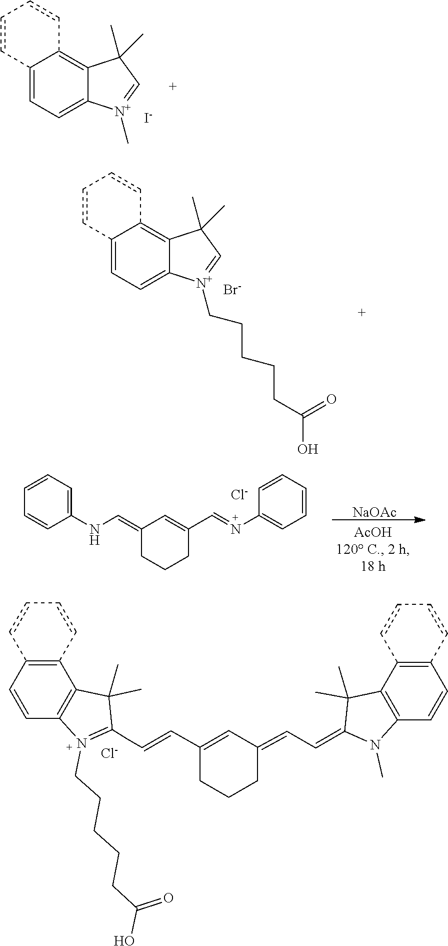

### Example 15 Synthesis of Bromine Activated Cy5 and Cy5.5

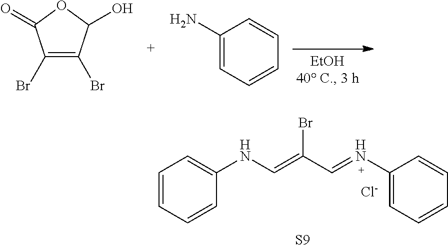

S9 was synthesized by adapting a previously described procedure.^{[89]} Briefly, mucobromic acid (2.974 g, 11.5 mmol, 1 eq) was dissolved in 20 mL of anhydrous ethanol and kept at room temperature. Separately, aniline (2.3 mL, 25 mmol, 2.2 eq) was mixed with 10 mL of anhydrous ethanol and this solution was added dropwise to mucobromic acid solution with vigorous stirring. The solution was gently heated to 40° C. and stirred for 3 hours, after which the solution was cooled to 0° C. in an ice bath and precipitated through the addition of 25 mL of diethyl ether. The resulting yellow solid was collected by vacuum filtration and washed 3× with 15 mL of Et₂O. The purified product was obtained as a yellow powder S9 (2.757 g, 7.2 mmol, 62.6% yield). ¹H NMR (Varian, 400 MHz, DMSO-d6), 5=11.53 ppm (br. s, 2H), 9.23 ppm (s, 2H), 7.61 ppm (d, 8.3 Hz, 4H), 7.53 ppm (t, 7.8 Hz, 4H), 7.36 ppm (t, 7.6 Hz, 2H). Identity of product was confirmed by LC-MS: m/z=303.0, [M]⁺.

This product was used for the synthesis of Cy5 or Cy5.5 bearing a bromine substituent on the middle carbon of the polyene chain using a method analogous to that used for unsubstituted Cy5 or Cy5.5. The conjugation of this bromine-functionalized cyanine to an analyte-sensing domain can then be performed using methodology comparable to that used for coupling chloride-functionalized Cy7 to an analyte sensing domain.

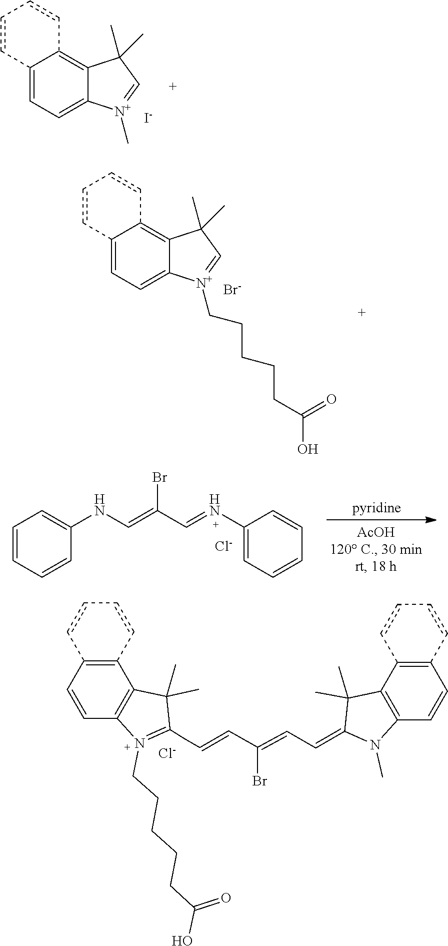

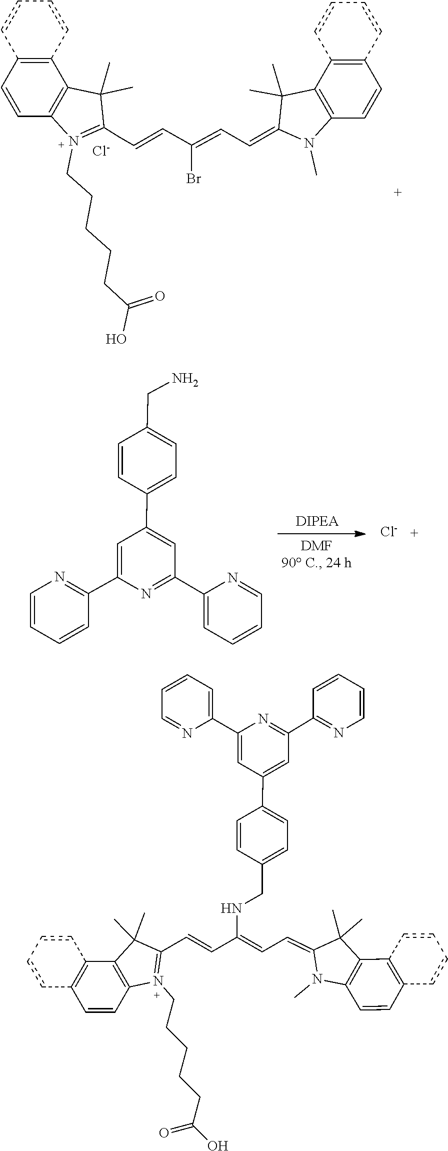

### Example 16 Synthesis of Pip-Cy7-PEG

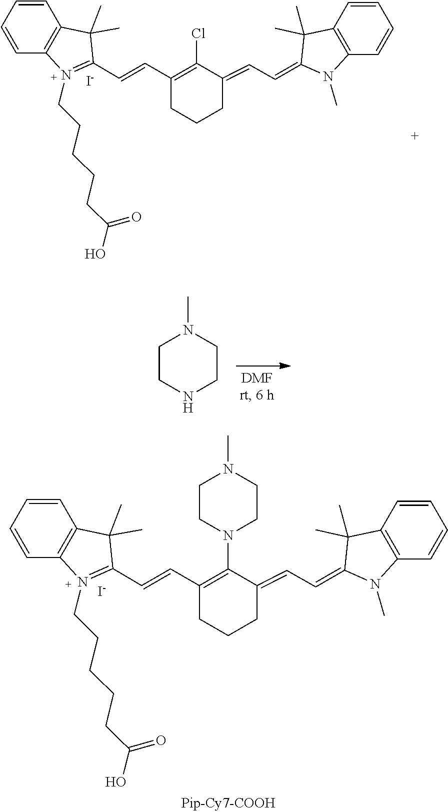

Pip-Cy7-COOH was first synthesized. Briefly, Cl-Cy7-COOH (0.200 g, 0.28 mmol, 1 eq) was dissolved in 6 mL of anhydrous DMF, and N-methylpiperazine (184 μL, 1.7 mmol, 6 eq) was added dropwise. The reaction mixture was covered in foil and stirred at room temperature for 6 h under argon. The reaction mixture was diluted in 40 mL DCM and washed with 2×20 mL portions of dH₂O, followed by 1×20 mL portion of sat. NaCl_{(aq)}. The organic layer was separated, dried over anhydrous Na₂SO₄, and solvent was removed in vacuo. The residue was purified by column chromatography on silica gel using a gradient of 5-10% methanol in DCM. The pure product was obtained as a reddish metallic solid of Pip-Cy7-COOH (0.123 g, 0.16 mmol, 56.5%). Identity of product was confirmed by LC-MS: m/z=647.4, [M]⁺.

PEGylation of Pip-Cy7-COOH was carried out using methods similar to those described in Example 9.

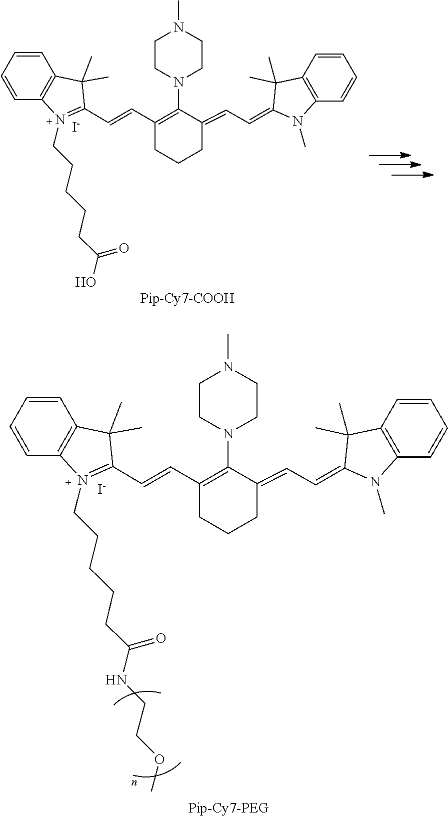

### Example 17 Measurement of pH and in Skin-Simulating Gels

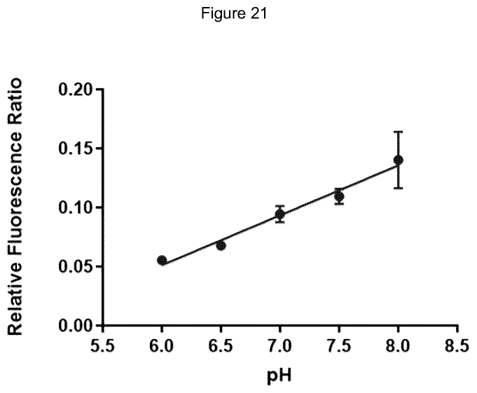

3% w/w solutions of agarose in acetate, phosphate, or tris buffer (set to pH values of 5-9) was prepared using a standard microwave, and 5 mL portions were dispensed into the wells of a 12-well cell culture plate (Sarstedt AG & Co., Nümbrecht, Germany). The wells were mixed thoroughly using a glass rod and allowed to cool and solidify for 2-3 h. Using a commercially available spring-loaded applicator generating an impact rate of 2 m·s⁻¹, an impact force of 1.6 N, and with a spring constant of 1 N·mm⁻¹ (values provided by the manufacturer, Micropoint Technologies Pte. Ltd., Singapore), microneedles containing Pip-Cy7-PEG and Cy7.5-PEG were applied to the gels for approximately 90 seconds, until the tips had fully dissolved. The fluorescence (λₑₓ=660 nm, λₑₘ=730 nm for Pip-Cy7-PEG; λₑₓ=765 nm, λₑₘ=810 nm for Cy7.5-PEG) of the gels was then monitored on a Spark® multimode fluorescence microplate reader. The results are shown in FIG. 21. FIG. 21 shows a clear correlation between the pH value of the medium and the measured fluorescence ratio.

### Example 18 Synthesis of Na⁺ Sensor Comprising Aza-Crown Ether

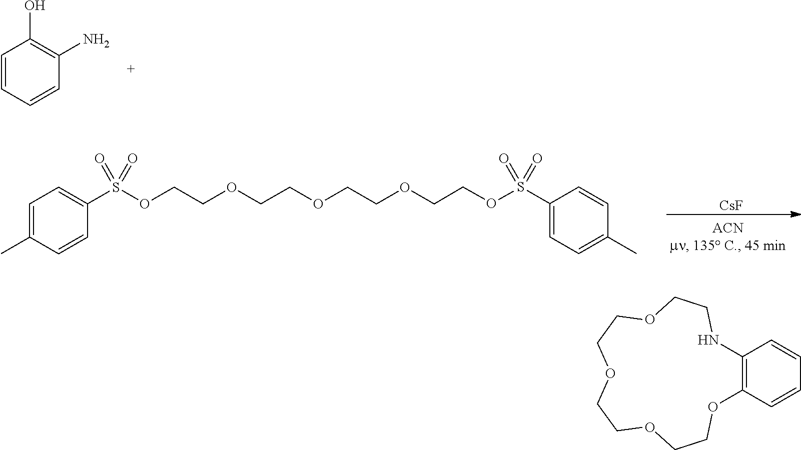

Benzo-15-aza-crown-5 was synthesized by adapting a previously described procedure.^{[91]} Briefly, 2-aminophenol (0.327 g, 3.0 mmol, 1 eq) and cesium fluoride (1.823 g, 12.0 mmol, 4 eq) were added to 15 mL of dry acetonitrile. Separately, tetraethylene glycol di(p-toluenesulfonate) (1.508 g, 3.0 mmol, 1 eq) was dissolved in 2 mL acetonitrile and added to the previous solution dropwise with continuous stirring. The reaction mixture was placed under argon atmosphere, sealed, and heated to 135° C. under microwave irradiation for 45 minutes. Solvent was removed in vacuo and the residue was dissolved in 100 mL of DCM and washed with a 50 mL portion of distilled water, followed by 50 mL of saturated sodium bicarbonate solution (NaHCO_{3(aq)}), 50 mL of distilled water, and 50 mL of saturated NaCl_{(aq)}. The organic layer was separated and dried over anhydrous sodium sulfate (Na₂SO₄), then filtered and the solvent was removed in vacuo to give the crude product, which was purified by column chromatography on silica gel using a gradient of 20-60% ethyl acetate in hexanes. The pure product was obtained as a waxy brown solid of benzo-15-aza-crown-5 (0.271 g, 1.01 mmol, 33.8%). ¹H NMR (Varian, 400 MHz, DMSO-d6): δ=6.79 ppm (t, 2H), 6.55 ppm (t, 2H), 4.96 ppm (br. s, 1H), 4.01 ppm (m, 2H), 3.72 ppm (m, 2H), 3.67 ppm (t, 2H), 3.57 ppm (m, 8H), 3.11 ppm (q, 2H). Identity of product was confirmed by LC-MS: m/z=268.2, [M+1]⁺.

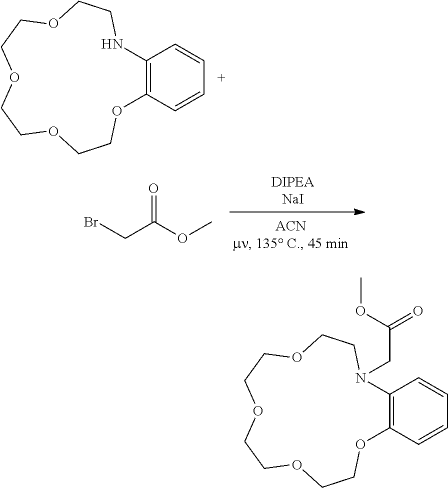

N-methylacetate-aza-benzo-15-crown-5 was synthesized by adapting a previously described procedure.^{[1]} Briefly, benzo-15-aza-crown-5 (0.267 g, 1.0 mmol, 1 eq) and sodium iodide (0.150 g, 1.0 mmol, 1 eq) were added to 3.5 mL of dry acetonitrile. Then, diisopropylethylamine (DIPEA) (871 μL, 5.0 mmol, 5 eq) and methyl bromoacetate (189 μL, 2.0 mmol, 2 eq) were added and the reaction mixture was placed under argon atmosphere, sealed, and heated to 135° C. under microwave irradiation for 45 minutes. The reaction mixture was diluted in 40 mL of DCM and washed with a 50 mL portion of 1% acetic acid, a 50 mL portion of distilled water, and a 50 mL portion of saturated NaCl_{(aq)}. The organic layer was separated and dried over anhydrous sodium sulfate (Na₂SO₄), then filtered and the solvent was removed in vacuo to give the crude product, which was purified by column chromatography on silica gel using a gradient of 0-5% methanol in DCM. The pure product was obtained as a yellow oil of N-methylacetate-aza-benzo-15-crown-5 (0.180 g, 0.53 mmol, 52.9%). Identity of product was confirmed by LC-MS: m/z=340.1, [M+1]⁺.

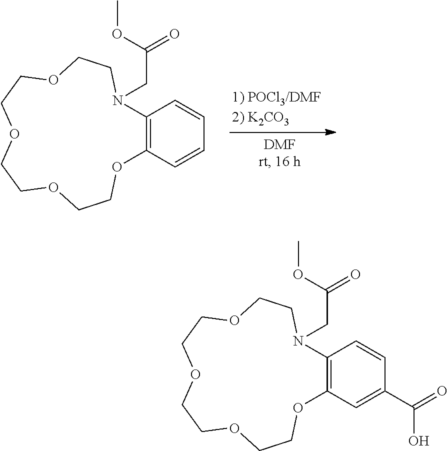 [SMILES: COC(=O)CN1CCOCCOCCOCCOc2ccccc21 ; COC(=O)CN1CCOCCOCCOCCOc2cc(C(=O)O)ccc21]

N-methylacetate-aza-15-benzocrown-5-aldehyde is synthesized by adapting a previously described procedure.^{[91]} Briefly, a solution of Vilsmeier reagent is prepared by the dropwise addition of phosphorus oxychloride (0.5 mL, 5 mmol, 5 eq) into 2.5 mL of dry N,N-dimethylformamide (DMF). Separately, N-methylacetate-aza-benzo-15-crown-5 (0.339 g, 1.0 mmol, 1 eq) is dissolved in 1 mL DMF and added to the solution of Vilsmeier reagent. The reaction mixture is stirred under argon atmosphere for 16 h, then poured into 40 mL of ice-cold saturated potassium carbonate (K₂CO₃) solution. This mixture is extracted 3× with 30 mL portions of DCM, then the organic layer is separated and dried over anhydrous sodium sulfate (Na₂SO₄). The solution is then filtered and the solvent is removed in vacuo to give the crude product, which is purified by column chromatography on silica gel using a gradient of 0-5% methanol in DCM. N-methylacetate-aza-15-benzocrown-5-aldehyde is obtained after solvent removal.

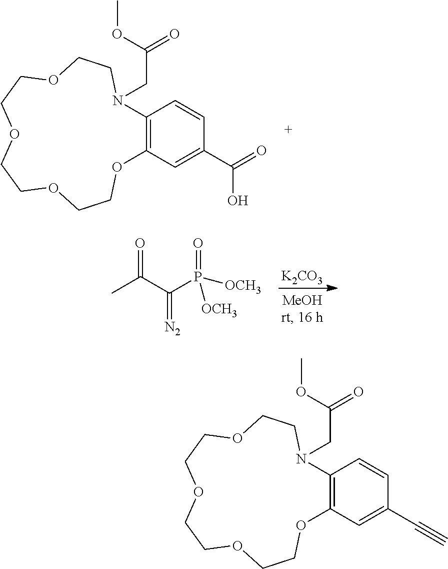

N-methylacetate-aza-4-ethynylbenzo[15]crown-5 is synthesized by adapting a previously described procedure.^{[92]} Briefly, N-methylacetate-aza-15-benzocrown-5-aldehyde (0.367 g, 1 mmol, 1 eq) and K₂CO₃ (0.276 g, 2 mmol, 2 eq) are added to 15 mL of dry methanol, and a 10% solution of dimethyl-1-diazo-2-oxopropylphosphonate in acetonitrile (2.93 mL, 1.2 mmol, 1.2 eq) is added dropwise. The reaction mixture is stirred under argon at room temperature for 20 h, then diluted with 20 mL of DCM. The mixture is washed 3× with 20 mL portions of distilled water, then the organic layer is separated and dried over anhydrous sodium sulfate (Na₂SO₄). The solution is then filtered and the solvent is removed in vacuo to give the crude product which is purified by column chromatography on silica gel using a gradient of 0-5% methanol in DCM. N-methylacetate-aza-4-ethynylbenzo[15]crown-5 is obtained.

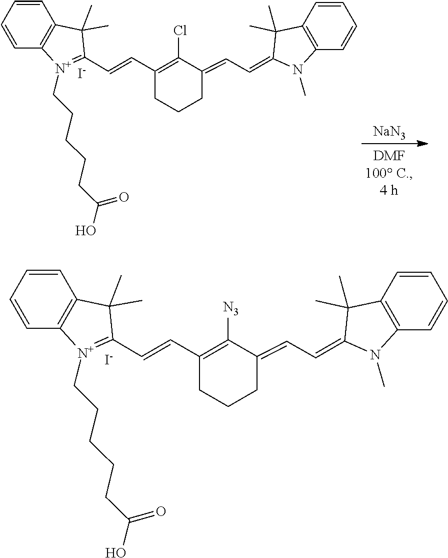 [SMILES: CN1/C(=C/C=C2\CCCC(/C=C/C3=[N+](CCCCCC(=O)O)c4ccccc4C3(C)C)=C2Cl)C(C)(C)c2ccccc21 ; [I-] ; CN1/C(=C/C=C2\CCCC(/C=C/C3=[N+](CCCCCC(=O)O)c4ccccc4C3(C)C)=C2N=[N+]=[N-])C(C)(C)c2ccccc21 ; [I-] ; "NaN3"]

Cy7-azide-COOH is synthesized by adapting a previously described procedure.^{[93]} Briefly, Cl-Cy7-COOH (0.071 g, 0.1 mmol, 1 eq) is dissolved in 1 mL of dry DMF, after which sodium azide (0.039 g, 0.6 mmol, 6 eq) was added. The reaction mixture is sealed under argon atmosphere and stirred at 100° C. for 4 h. The reaction mixture is diluted in 40 mL DCM and washed with 2×20 mL portions of distilled water, followed by 1×20 mL portion of sat. NaCl_{(aq)}. The organic layer is separated, dried over anhydrous Na₂SO₄, and solvent is removed in vacuo. The residue is purified by column chromatography on silica gel using a gradient of 5-10% methanol in DCM. The pure product is obtained as Cy7-azide-COOH.

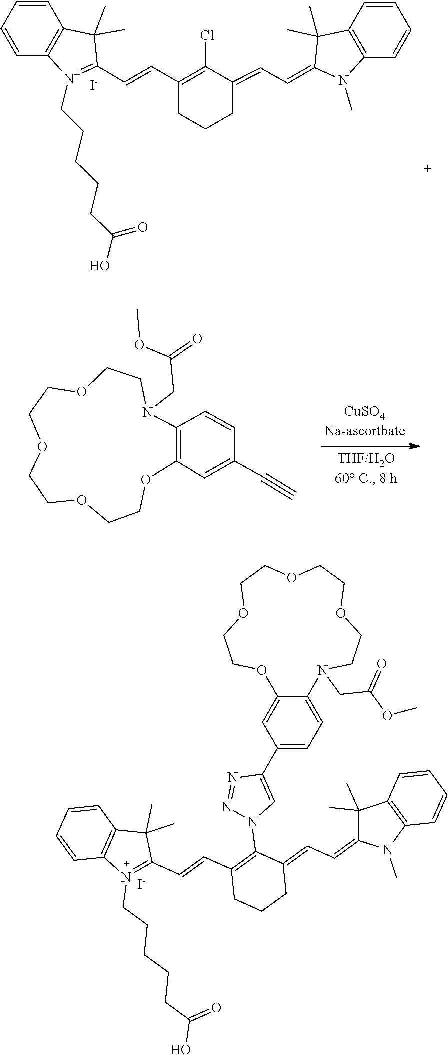

N-methylacetate-aza-15-benzocrown-5-Cy7 is synthesized by adapting a previously described procedure.^{[92]} Briefly, N-methylacetate-aza-4-ethynylbenzo[15]crown-5 (0.182 g, 0.5 mmol, 1 eq) and Cy7-azide-COOH (0.359 g, 0.5 mmol, 1 eq) are added to 10 mL of a 2:1 mixture of DMF and distilled water, followed by 6.4 mg of copper sulfate pentahydrate (CuSO₄·5H₂O) and 9.9 mg of sodium ascorbate. The reaction mixture is stirred at 60° C. for 48 h, then diluted with 40 mL of DCM. The organic layer is separated, dried over anhydrous Na₂SO₄, and solvent is removed in vacuo. The residue is purified by column chromatography on silica gel using a gradient of 5-10% methanol in DCM. The pure product is obtained as N-methylacetate-aza-15-benzocrown-5-Cy7.

### Example 19 In Vivo Testing of pH and Sodium Sensing of Sensors

The sodium and pH (proton) sensors of the present disclosure can be tested according to methods similar to the ROS and pH sensing described in examples above. A rat model based in vivo is described below for sodium and acidosis testing.

Male Sprague-Dawley rats (Charles River, St-Constant, QC) with weight ranging from 300-350 g at delivery are used for this study. Following arrival in the animal facility, all animals are subjected to a general health evaluation. An acclimation period of 5 days is allowed before the beginning of the study. Each animal cage is equipped with a manual water distribution system. A standard certified commercial rodent diet is provided ad libitum. Tap water is provided ad libitum at all times.

For induction of acidosis, animals are provided 0.28 M NH4Cl/0.5% sucrose (acidosis) or 0.5% sucrose (control) water ad libitum. Starting one day before induction of acidosis, the blood and urine pH of the rats are measured once daily in the morning.

For sodium dysfunction model, the animals are provided with NaCl/0.5% sucrose (sodium dysfunction) or 0.5% sucrose (control) water ad libitum. Starting one day before induction of the sodium dysfunction, the blood and urine sodium level of the rats are measured once daily in the morning.

Rats dorsal skin is shaved and depilated under isoflurane-induced anesthesia using an electric razor and depilating cream (Nair, Church & Dwight) the day before experimentation. Rats are extensively rinsed to prevent burning with residual depilating agent.

After, MNs are applied under anesthesia using a commercially available spring-loaded applicator for 2 minutes for each application. Three MN arrays are applied at the same time on different sites of the dorsal skin. MNs are removed, and the application sites are scanned for fluorescence using the portable fluorescence reader. On the first day, sites are read after 0.5, 1, 4, and 6 h. On the second day, new MNs are applied and read immediately or 6 h after application. New MNs are also applied on day 3 and 4 post-induction of acidosis.

Readings of MNs fluorescence results are as described above.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS OF DOCUMENTS REFERENCED HEREIN

[1] S. S. Gambhir, T. J. Ge, O. Vermesh, R. Spitler, Sci. Transl. Med. 2018, 10, eaao3612.
[2] X. A. Cambronne, M. L. Stewart, D. Kim, A. M. Jones-Brunette, R. K. Morgan, D. L. Farrens, M. S. Cohen, R. H. Goodman, Science 2016, 352, 1474.
[3] R. M. Williams, C. Lee, T. V. Galassi, J. D. Harvey, R. Leicher, M. Sirenko, M. A. Dorso, J. Shah, N. Olvera, F. Dao, D. A. Levine, D. A. Heller, Sci. Adv. 2018, 4, eaaq1090.
[4] B. Q. Tran, P. R. Miller, R. M. Taylor, G. Boyd, P. M. Mach, C. N. Rosenzweig, J. T. Baca, R. Polsky, T. Glaros, J. Proteome Res. 2018, 17, 479.
[5] M. Gilanyi, C. Ikrenyi, J. Fekete, K. Ikrenyi, A. G. Kovach, Am. J. Physiol. Renal Physiol. 1988, 255, 513.
[6] S. N. Thennadil, J. L. Rennert, B. J. Wenzel, K. H. Hazen, T. L. Ruchti, M. B. Block, Diabetes Technol. Ther. 2001, 3, 357.
[7] N. Fogh-Andersen, B. M. Altura, B. T. Altura, O. Siggaard-Andersen, Clin. Chem. 1995, 41, 1522.
[8] P. P. Samant, M. M. Niedzwiecki, N. Raviele, V. Tran, J. Mena-Lapaix, D. I. Walker, E. I. Felner, D. P. Jones, G. W. Miller, M. R. Prausnitz, Sci. Transl. Med. 2020, 12, eaaw0285.
[9] S. Babity, M. Roohnikan, D. Brambilla, Small 2018, 14, 1803186.
[10] H. Chang, M. Zheng, X. Yu, A. Than, R. Z. Seeni, R. Kang, J. Tian, D. P. Khanh, L. Liu, P. Chen, C. Xu, Adv. Mater. 2017, 29, 1702243.
[11] P. R. Miller, S. A. Skoog, T. L. Edwards, D. R. Wheeler, X. Xiao, S. M. Brozik, R. Polsky, R. J. Narayan, J. Vis. Exp. 2012, 1, e4067.
[12] J. Yu, Y. Zhang, Y. Ye, R. DiSanto, W. Sun, D. Ranson, F. S. Ligler, J. B. Buse, Z. Gu, Proc. Natl. Acad. Sci. U.S.A 2015, 112, 8260.
[13] J. Yu, C. Qian, Y. Zhang, Z. Cui, Y. Zhu, Q. Shen, F. S. Ligler, J. B. Buse, Z. Gu, Nano Lett. 2017, 17, 733.
[14] G. Chen, J. Yu, Z. Gu, J. Diabetes Sci. Technol. 2018, 13, 41.
[15] P. P. Samant, M. R. Prausnitz, Proc. Natl. Acad. Sci. U.S.A 2018, 115, 4583.
[16] M. Wang, L. Hu, C. Xu, Lab Chip 2017, 17, 1373.
[17] D. Brambilla, S. T. Proulx, P. Marschalkova, M. Detmar, J.-C. Leroux, Small 2016, 12, 1053.
[18] A. K. Polomska, S. T. Proulx, D. Brambilla, D. Fehr, M. Bonmarin, S. Brsndli, M. Meboldt, C. Steuer, T. Vasileva, N. Reinke, J.-C. Leroux, M. Detmar, JCI Insight 2019, 4, 126515.
[19] S. Babity, A. K. Polomska, F. Couture, M. Bonmarin, D. Fehr, M. Detmar, D. Brambilla, J. Control. Release 2020, 327, 350.
[20] K. J. McHugh, L. Jing, S. Y. Severt, M. Cruz, M. Sarmadi, H. S. N. Jayawardena, C. F. Perkinson, F. Larusson, S. Rose, S. Tomasic, T. Graf, S. Y. Tzeng, J. L. Sugarman, D. Vlasic, M. Peters, N. Peterson, L. Wood, W. Tang, J. Yeom, J. Collins, P. A. Welkhoff, A. Karchin, M. Tse, M. Gao, M. G. Bawendi, R. Langer, A. Jaklenec, Sci. Transl. Med. 2019, 11, eaay7162.
[21] K. van der Maaden, E. Sekerdag, W. Jiskoot, J. Bouwstra, AAPS J. 2014, 16, 681.
[22] E. M. Vicente-Pérez, H. L. Quinn, E. McAlister, S. O'Neill, L.-A. Hanna, J. G. Barry, R. F. Donnelly, Pharm. Res. 2016, 33, 3072.
[23] C. C. Winterbourn, Nat. Chem. Biol. 2008, 4, 278.
[24] K. Apel, H. Hirt, Annu. Rev. Plant Biol. 2004, 55, 373.
[25] H. Sakurai, H. Yasui, Y. Yamada, H. Nishimura, M. Shigemoto, Photochem. Photobiol. Sci. 2005, 4,715.
[26] P. Talalay, J. W. Fahey, Z. R. Healy, S. L. Wehage, A. L. Benedict, C. Min, A. T. Dinkova-Kostova, Proc. Natl. Acad. Sci. U.S.A 2007, 104, 17500.
[27] J. Komatsu, H. Koyama, N. Maeda, Y. Aratani, Inflamm. Res. 2006, 55, 200.
[28] Z. Guo, B. Zhou, W. Li, X. Sun, D. Luo, J. Biomed. Res. 2012, 26, 365.
[29] J. L. M. Hawk, G. M. Murphy, C. A. Holden, Br. J. Dermatol. 1988, 118, 27.
[30] I. Strickland, L. E. Rhodes, B. F. Flanagan, P. S. Friedmann, J. Invest. Dermatol. 1997, 108, 763.
[31] S. C. Dharmage, A. J. Lowe, M. C. Matheson, J. A. Burgess, K. J. Allen, M. J. Abramson, Allergy 2014, 69, 17.
[32] W. D. Boothe, J. A. Tarbox, M. B. Tarbox, in Management of Atopic Dermatitis, (Eds: E. Fortson, S. R. Feldman, L. C. Strowd), Springer, New York, 2017, Ch. 2.
[33] N. Sivaranjani, S. V. Rao, G. Rajeev, J. Clin. Diagnostic Res. 2013, 7, 2683.
[34] H. Tsukahara, R. Shibata, Y. Ohshima, Y. Todoroki, S. Sato, N. Ohta, M. Hiraoka, A. Yoshida, S. Nishima, M. Mayumi, Life Sciences 2003, 72, 2509.
[35] H.-J. Kim, E. Lee, S.-H. Lee, M.-J. Kang, S.-J. Hong, Clin. Immunol. 2015, 161, 384.
[36] H. Y. Chang, D. I. Suh, S.-. Yang, M.-J. Kang, S.-Y. Lee, E. Lee, I. A. Choi, K.-S. Lee, Y.-J. Shin, Y. H. Shin, Y. H. Kim, K. W. Kim, K. Ahn, H.-S. Won, S.-J. Choi, S.-Y. Oh, J.-Y. Kwon, Y. H. Kim, H. J. Park, K.-J. Lee, J. K. Jun, H.-S. Yu, S.-H. Lee, B. K. Jung, J.-W. Kwon, Y. K. Choi, N. Do, Y. J. Bae, H. Kim, W.-S. Chang, E.-J. Kim, J. K. Lee, S.-J. Hong, J. Allergy Clin. Immunol. 2016, 138, 468.
[37] E. Patelarou, G. Giourgouli, A. Lykeridou, E. Vrioni, N. Fotos, E. Siamaga, V. Vivilaki, H. Brokalaki, Nutr. Rev. 2011, 69, 627.
[38] A. M. Wolf, K. Nishimaki, N. Kamimura, S. Ohta, J. Investig. Dermatol. 2014, 134, 1701.
[39] K. Kundu, S. F. Knight, N. Willett, S. Lee, W. R. Taylor, N. Murthy, Angew. Chem. Int. Ed. Engl. 2009, 48, 299.
[40] S. Suri, S. M. Lehman, S. Selvam, K. Reddie, S. Maity, N. Murthy, A. J. Garcia, J. Biomed. Mater. Res. A. 2015, 103, 76.
[41] J. V. Frangioni, Curr. Opin. Chem. Biol. 2003, 7, 626.
[42] R. F. Donnelly, M. J. Garland, D. I. J. Morrow, K. Migalska, T. R. R. Singh, R. Majithiya, A. D. Woolfson, J. Control. Release 2010, 147, 333.
[43] A. Bigdeli, F. Ghasemi, S. Abbasi-Moayad, M. Shahrajabian, N. Fahimi-Kashani, S. Jafarinejad, M. A. F. Nejad, M. R. Hormozi-Nezhad, Anal. Chim. Acta 2019, 1079, 30.
[44] R. M. SAnchez-Martin, M. Cuttle, S. Mittoo, M. Bradley, Angew. Chem. Int. Ed. Engl. 2006, 45, 5472.
[45] W. Lin, L. Yuan, W. Tan, J. Feng, L. Long, Chem. Eur. J. 2009, 15, 1030.
[46] Z.-H. Xu, H.-W. Wang, X.-F. Hou, W.-L. Xu, T.-C. Xiang, C.-Z. Wu, Sens. Actuators B Chem. 2014, 201, 469.
[47] K. Kim, M. Lee, H. Park, J.-H. Kim, S. Kim, H. Chung, K. Choi, I.-S. Kim, B. L. Seong, I. C. Kwon, J. Am. Chem. Soc. 2006, 128, 3490.
[48] C. Haritoglou, M. Kernt, P. Laubichler, H. Langhals, K. Eibl, A. Varja, S. Thaler, A. Kampik, Graefe Arch. Clin. Exp. Ophthalmol. 2012, 250, 829.
[49] R. W. Gao, N. Teraphongphom, E. de Boer, N. S. van den Berg, V. Divi, M. J. Kaplan, N. J. Oberhelman, S. S. Hong, S. Capes, A. D. Colevas, E. L. Rosenthal, Theranostics 2018, 8, 2488.
[50] D. Li, J. Zhang, C. Chi, X. Xiao, J. Wang, L. Lang, I. Ali, G. Niu, L. Zhang, J. Tian, N. Ji, Z. Zhu, X. Chen, Theranostics 2018, 8, 2508.
[51] G. R. Cherrick, S. W. Stein, C. M. Leevy, C. S. Davidson, J. Clin. Invest. 1960, 39, 592.
[52] H. von Berlepsch, C. Böttcher, A. Ouart, C. Burger, S. Dshne, S. Kirstein, J. Phys. Chem. B 2000, 104, 5255.
[53] L. I. Markova, V. L. Malinovskii, L. D. Patsenker, R. Hsner, Chem. Commun. 2013, 49, 5298.
[54] H. von Berlepsch, C. Böttcher, J. Phys. Chem. B 2015, 119, 11900.
[55] V. Kumar, D. S. Kalonia, AAPS PharmSciTech. 2006, 7, 47.
[56] S. Babity, D. Brambilla, Data in Brief 2020, 32, 106258.
[57] C. Walling, G. M. El-Taliawi, R. A. Johnson, J. Am. Chem. Soc. 1974, 96,133.
[58] C. Walling, Acc. Chem. Res. 1975, 8,125.
[59] W. A. Pryor, Ann. Rev. Physiol. 1986, 48, 657.
[60] E. Brillas, I. Sires, M. A. Oturan, Chem. Rev. 2009, 109, 6570.
[61] R. C. B. Castillo, J.-M. Fontmorin, W. Z. Tang, X. Dominguez-Benetton, M. Sillanpss, RSC Adv. 2018, 8, 5321.
[62] W. R. Wasylaschuk, P. A. Harmon, G. Wagner, A. B. Harman, A. C. Templeton, H. Xu, R. A. Reed, J. Pharm. Sci. 2007, 96,106.
[63] M. Ashraf-Khorassani, L. T. Taylor, K. C. Waterman, P. Narayan, D. R. Brannegan, G. L. Reid, Pharm. Dev. Technol. 2005, 10, 507.
[64] F. Haaf, A. Sanner, F. Straub, Polym. J. 1985, 17, 143.
[65] C. Brough, D. A. Miller, J. M. Keen, S. C. Kucera, R. O. Williams, AAPS PharmSciTech. 2016, 17, 167.
[66] S. Liu, M.-N. Jin, Y.-S. Quan, F. Kamiyama, H. Katsumi, T. Sakane, A. Yamamoto, J. Control. Release 2012, 161, 933.
[67] S. P. Davis, B. J. Landis, Z. H. Adams, M. G. Allen, M. R. Prausnitz, J. Biomech. 2004, 37, 1155.
[68] J. W. Lee, J.-H. Park, M. R. Prausnitz, Biomaterials 2008, 29, 2113.
[69] K. Lee, C. Y. Lee, H. Jung, Biomaterials 2011, 32, 3134.
[70] S. G. Lee, J. H. Jeong, K. M. Lee, K. H. Jeong, H. Yang, M. Kim, H. Jung, S. Lee, Int. J. Nanomedicine 2014, 9, 289.
[71] K. Ita, Biomed. Pharmacother. 2017, 93, 1116.
[72] S. Bolte, F. P. Cordelibres, J. Microsc. 2006, 224, 213.
[73] S. T. Proulx, P. Luciani, A. Christiansen, S. Karaman, K. S. Blum, M. Rinderknecht, J.-C. Leroux, M. Detmar, Biomaterials 2013, 34, 5128.
[74] D. Zhang, D. B. Das, C. D. Rielly, J. Pharm Sci. 2014, 103, 613.
[75] W. W. Koelmans, G. Krishnamoorthy, A. Heskamp, J. Wissink, S. Misra, N. Tas, Mech. Eng. Res. 2013, 3, 51.
[76] S. Hönzke, L. Wallmeyer, A. Ostrowski, M. Radbruch, L. Mundhenk, M. Schsfer-Korting, S. Hedtrich, J. Invest. Dermatol. 2016, 136, 631.
[77] A. Elpelt, S. Albrecht, C. Teutloff, M. Huging, S. Saiedpour, S. B. Lohan, M. C. Meinke, Chem. Biol. Interact. 2019, 310, 108752.
[78] L. Wallmeyer, D. Lehnen, N. Eger, M. Sochorová, L. Opaĺka, A. Kováĉik, K. Vávrová, S. Hedtrich, J. Dermatol. Sci. 2015, 80, 102.
[79] L. Wallmeyer, K. Dietert, M. SochorovA, A. D. Gruber, B. Kleuser, K. VAvrovA, S. Hedtrich, Sci. Rep. 2017, 7, 774.
[80] K. Vávrová, D. Henkes, K. Strüver, M. Sochorová, B. Ŝkolová, M. Y. Witting, W. Friess, S. Schreml, R. J. Meier, M. Schäfer-Korting, J. W. Fluhr, S. Kuchler, J. Invest. Dermatol. 2014, 134, 746.
[81] M. V. Kvach, A. V. Ustinov, I. A. Stepanova, A. D. Malakhov, M. V. Skorobogatyi, V. V. Shmanai, V. A. Korshun, Eur. J. Org. Chem. 2008, 2008, 2107.
[82] D. Andina, D. Brambilla, N. Munzinger, J. Frei, C. Zivko, J.-C. Leroux, P. Luciani, Chem. Eur. J. 2017, 23, 4765.
[83] E. Larrañeta, R. E. M. Lutton, A. J. Brady, E. M. Vicente-Pérez, A. D. Woolfson, T. R. R. Singh, R. F. Donnelly, Macromol. Mater. Eng. 2015, 300, 586.
[84] J. Sangster, J. Phys. Chem. Ref. Data. 1989, 18, 1111.
[85] B. Tang, F. Yu, P. Li, L. Tong, X. Duan, T. Xie, X. Wang, J. Am. Chem. Soc. 2009, 131, 3016.
[86] M. V. Kvach, A. V. Ustinov, I. A. Stepanova, A. D. Malakhov, M. V. Skorobogatyi, V. V. Shmanai, V. A. Korshun, Eur. J. Org. Chem. 2008, 2008, 2107.
[87] G. A. Reynolds, K. H. Drexhage, J. Org. Chem. 1977, 42, 885.
[88] N. Narayanan, G. Patonay, J. Org. Chem. 1995, 60, 2391.
[89] J. Zhang, B. Yu, L. Ning, X. Zhu, J. Wang, Z. Chen, X. Liu, X. Yao, X. Zhang, H. Zhang, Eur. J. Org. Chem. 2015, 2015, 1711.
[90] O. P. Kryatova, A. G. Kolchinski, E. V. Rybak-Akimova, Tetrahedron 2003, 59, 231.
[91] V. V. Martin, A. Rothe, K. R. Gee, Fluorescent metal ion indicators based on benzoannelated crown systems: a green fluorescent indicator for intracellular sodium ions. Bioorg. Med. Chem. Lett. 15 (2005), 1851-1855. DOI: 10.1016/j.bmcl.2005.02.017.
[92] T. Schwarze, H. Müller, D. Schmidt, J. Riemer, H.-J. Holdt, Design of Na+-selective fluorescent probes: A systematic study of the Na+-complex stability and the Na+/K+ selectivity in acetonitrile and water. Chem. Eur. J. 23 (2017), 7255-7263. DOI: 10.1002/chem.201605986.
[93] A. Ouerghui, H. Elamari, M. Dardouri, S. Ncib, F. Meganem, C. Girard, Chemical modifications of poly(vinyl chloride) to poly(vinyl azide) and “clicked triazole bearing groups for application in metal cation extraction. React. Funct. Polym. 100 (2016), 191-197. DOI: 10.1016/j.reactfunctpolym.2016.01.016.

1. A ratiometric detection composition comprising
a reference dye, the reference dye being a first cyanine dye that is PEGylated; and
a sensor dye, the sensor dye being a second cyanine dye that is PEGylated and that comprises an analyte sensing moiety;
wherein the reference dye is inert towards the analyte;
wherein an emission maximum wavelength of the reference dye and an emission maximum wavelength of the sensor dye are at least 50 nm apart;
wherein the first cyanine dye and the second cyanine dye are PEGylated with PEG groups of substantially the same molecular weight.

2. The ratiometric detection composition of claim 1, wherein the reference dye has a structure of Formula I 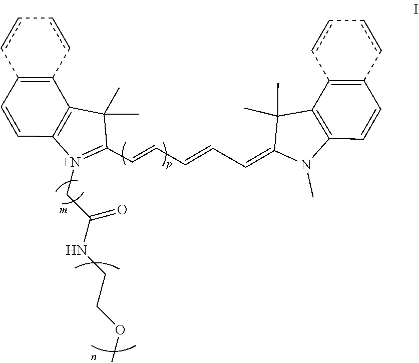
or a salt thereof,
wherein p is an integer from 1 to 3, m is an integer from 2 to 8, n is an integer from 50 to 900.

3. The ratiometric detection composition of claim 1 or 2, wherein the analyte sensing moiety is a reactive oxygen species (ROS) sensing moiety, and the sensor dye has a structure of Formula II 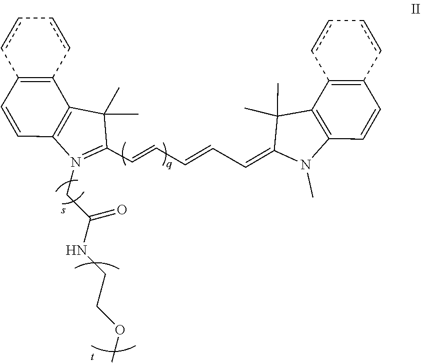
or a salt thereof,
wherein q is an integer from 1 to 3, s is an integer from 2 to 8, t is an integer from 50 to 900, and wherein the 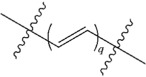
of the compound is optionally substituted with C₁₋₆alkyl or C₁₋₆cycloalkyl.

4. The ratiometric detection composition of claim 3, wherein p is 1, and/or m is 4 to 6, optionally 5.

5. The ratiometric detection composition of claim 3 or 4, wherein q is 2, and/or s is 4 to 6, optionally 5.

6. The ratiometric detection composition of any one of claims 3 to 5, wherein n and t are each independently about 50 to about 800, about 50 to about 700, about 50 to about 650, about 50 to about 600, about 50 to about 550, about 50 to about 500, about 50 to about 450, about 50 to about 400, about 50 to about 350, about 50 to about 300, about 80 to about 250, about 80 to about 200, about 90 to about 150, about 90 to about 130, or about 110.

7. The ratiometric detection composition of claim 1 or 2, wherein the sensor dye has a structure of Formula IIIa or IIIb 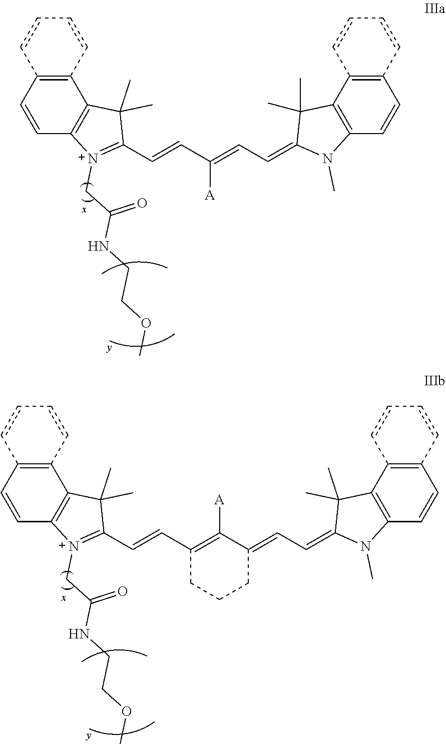
or a salt thereof,
wherein each x is independently an integer from 2 to 8, each y is independently an integer from 50 to 900;
wherein each A is independently the analyte sensing moiety.

8. The ratiometric composition of any one of claims 1, 2, and 7, wherein the analyte sensing moiety is selected from pH sensing moiety or cation sensing moiety.

9. The ratiometric composition of claim 8, wherein the pH sensing moiety comprises a terpyridine or a piperazine.

10. The ratiometric composition of claim 9, wherein the sensor dye has a structure of Formula IIIb1 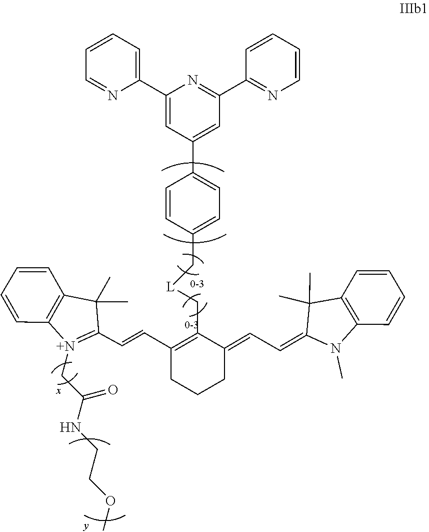
or a salt thereof,
wherein x is an integer from 2 to 8, y is an integer from 50 to 900, L is O, NH, or N(C₁₋₃alkyl).]

11. The ratiometric detection composition of claim 8, wherein the cation sensing moiety comprises a crown ether and/or an aza crown ether, optionally the crown ether being selected from 12-crown-4, 15-crown-5, 18-crown-6, and aza-15-crown-5.

12. The ratiometric detection composition of claim 11, wherein the cation sensing moiety is Li⁺ sensing and the crown ether is 12-crown-4.

13. The ratiometric detection composition of claim 12, wherein the sensor dye has a structure of Formula IIIb2 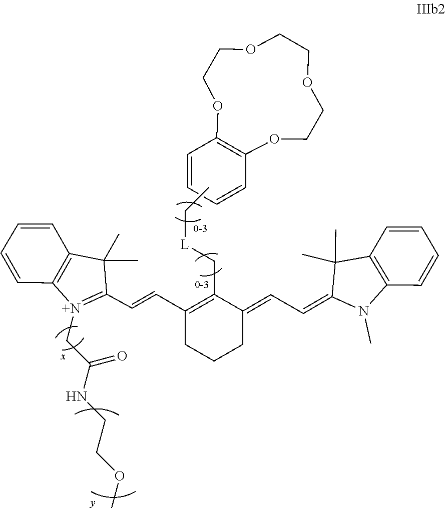
or a salt thereof,
wherein x is an integer from 2 to 8, y is an integer from 50 to 900, and L is O, NH, N(C₁₋₃alkyl), or phenyl.

14. The ratiometric detection composition of claim 11, wherein the cation sensing moiety is Na⁺ sensing and the crown ether is 15-crown-5.

15. The ratiometric detection composition of claim 14, wherein the sensor dye has a structure of Formula IIIb3 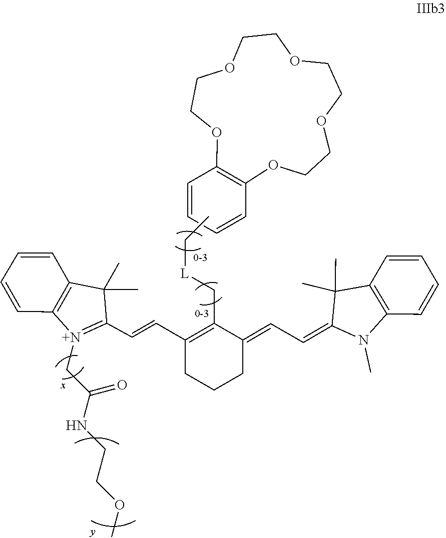
or a salt thereof,
wherein x is an integer from 2 to 8, y is an integer from 50 to 900, and L is O, NH, N(C₁₋₃alkyl), or phenyl.

16. The ratiometric detection composition of claim 11, wherein the cation sensing moiety is K⁺ sensing and the crown ether is 18-crown-6.

17. The ratiometric detection composition of claim 16, wherein the sensor dye has a structure of Formula IIIb4 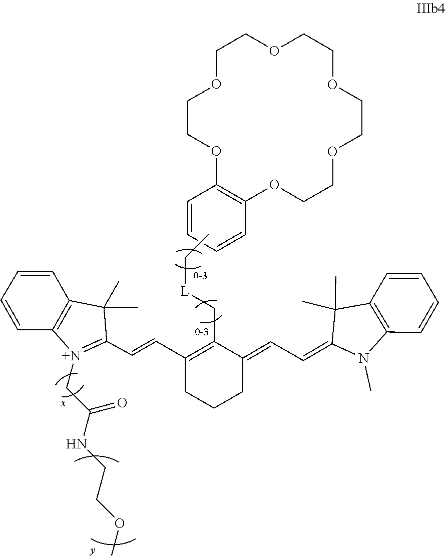
or a salt thereof,
wherein x is an integer from 2 to 8, y is an integer from 50 to 900, and L is O, NH, N(C₁₋₃alkyl), or phenyl.

18. The ratiometric detection composition of claim 9, wherein the sensor dye has a structure of Formula IIIb5 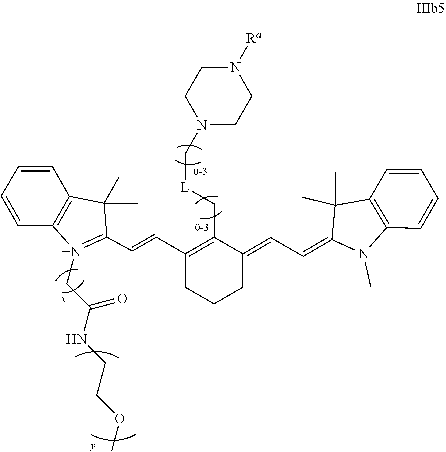
or a salt thereof,
wherein x is an integer from 2 to 8, y is an integer from 50 to 900, L is absent, O, NH, N(C₁₋₃alkyl), phenyl, or heteroaryl, and R^{a} is C₁₋₆ alkyl.

19. The ratiometric detection composition of claim 18, wherein L is absent, and the sensor dye has a structure of

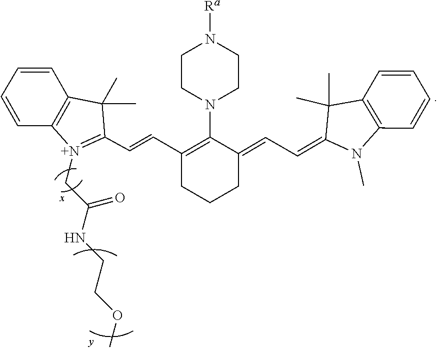

20. The ratiometric detection composition of claim 9, wherein the sensor dye has a structure of Formula IIIb6 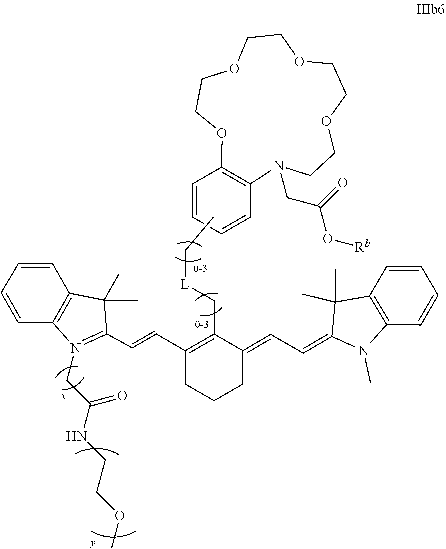
or a salt thereof
wherein x is an integer from 2 to 8, y is an integer from 50 to 900, L is O, NH, N(C₁₋₃alkyl), phenyl, or heteroaryl, and R^{b} is C₁₋₆ alkyl.

21. The ratiometric detection composition of claim 20, wherein the heteroaryl of L is triazole, optionally the sensor dye has a structure of 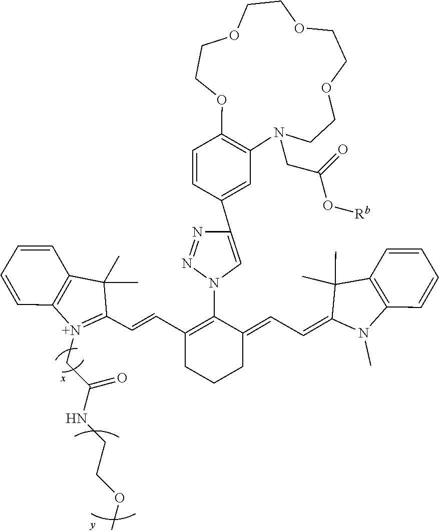
or a salt thereof.

22. The ratiometric detection composition of claim 20 or 21, wherein R^{b} is a C₁₋₃ alkyl, optionally R^{b} is methyl.

23. The ratiometric detection composition of any one of claims 7 to 22, wherein x is 4 to 6, optionally 5.

24. The ratiometric detection composition of any one of claims 7 to 23, wherein y is about 50 to about 800, about 50 to about 700, about 50 to about 650, about 50 to about 600, about 50 to about 550, about 50 to about 500, about 50 to about 450, about 50 to about 400, about 50 to about 350, about 50 to about 300, about 80 to about 250, about 80 to about 200, about 90 to about 150, about 90 to about 130, or about 110.

25. The ratiometric detection composition of any one of claims 7 to 23, wherein p is 2, m is 4 to 6, optionally 5, and/or n is about 50 to about 800, about 50 to about 700, about 50 to about 650, about 50 to about 600, about 50 to about 550, about 50 to about 500, about 50 to about 450, about 50 to about 400, about 50 to about 350, about 50 to about 300, about 80 to about 250, about 80 to about 200, about 90 to about 150, about 90 to about 130, or about 110.

26. The ratiometric detection composition of claim 1, wherein the reference dye is 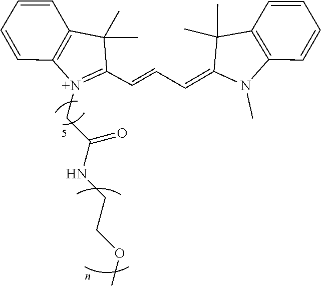
or a salt thereof,
wherein n is about 90 to about 130, optionally about 110.

27. The ratiometric detection composition of claim 1 or 26, wherein the sensor dye is 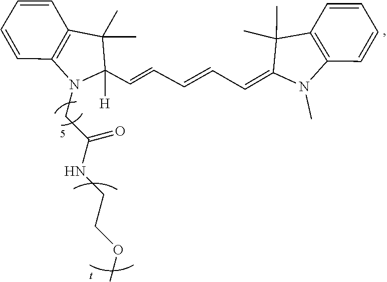
or a salt thereof,
wherein t is about 90 to about 130, optionally about 110.

28. The ratiometric detection composition of claim 1, wherein the reference dye is 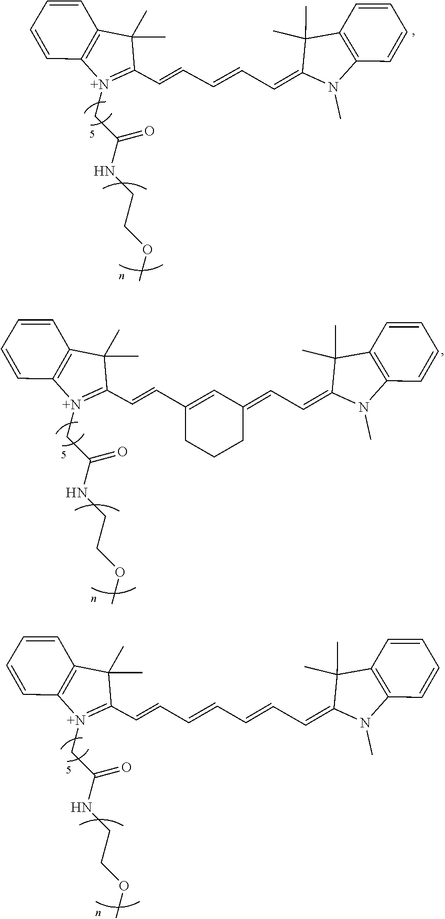
or a salt thereof,
wherein n is about 90 to about 130, optionally about 110.

29. The ratiometric detection composition of claim 1 or 28, wherein the sensor dye is selected from 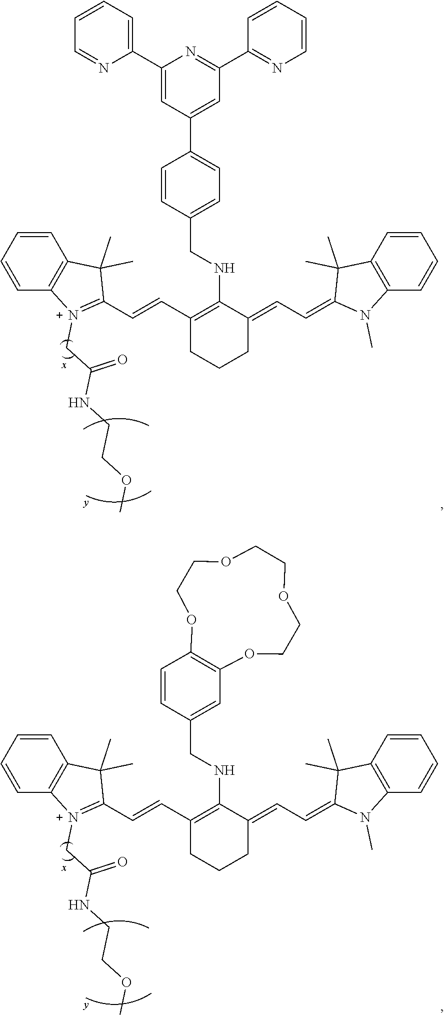 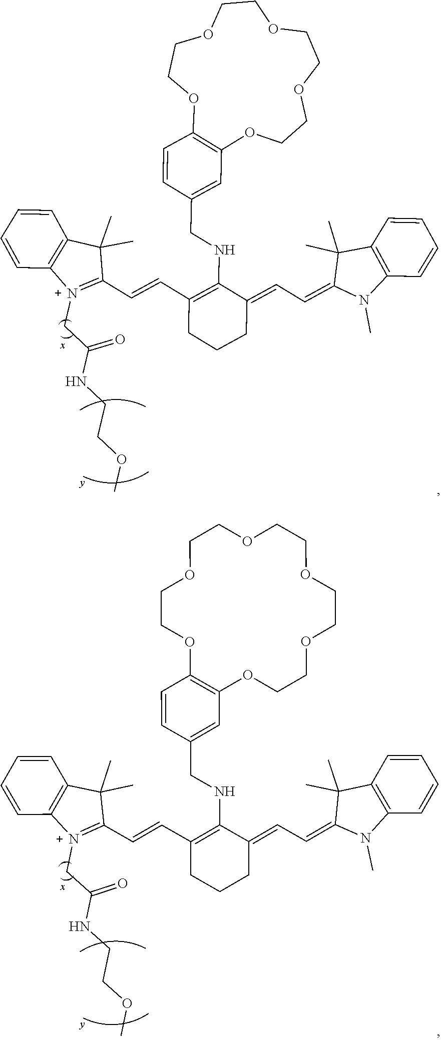 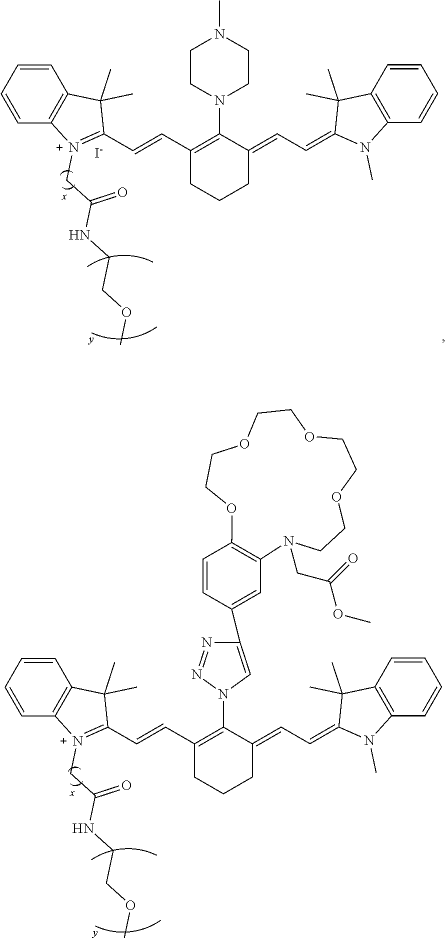
or salt thereof, wherein x is 5 and y is about 90 to about 130, optionally about 110.

30. The ratiometric detection composition of any one of claims 1 to 29 comprising a plurality of sensor dyes, each of which is independently as defined in any one of claims 1 to 26.

31. A reference dye compound as defined in any one of claims 2, 4, 26, and 28.

32. A sensor dye compound as defined in any one of claims 3, 5, 7 to 21, 27, and 29.

33. The ratiometric detection composition of any one of claims 1 to 32 or a reference dye compound of claim 31, or a sensor dye compound of claim 32 for use in a ratiometric detection, measurement and/or monitoring of an analyte.

34. The ratiometric detection composition of any one of claims 1 to 30 comprising a plurality of sensor dyes, a reference dye compound of claim 31, or a plurality of sensor dye compounds of claim 32 for use in a ratiometric detection, measurement and/or monitoring of a plurality of analytes.

35. A microneedle array comprising
a backing layer, and
a plurality of microneedles each having a first end and a second sharpened end for penetration into skin, the plurality of microneedles extending outwardly from the backing layer at the first end;
wherein the microneedles are formed with a biodegradable polymer and comprise a ratiometric detection composition as defined in any one of claims 1 to 30 that is incorporated in the biodegradable polymer;
wherein the plurality of microneedles is dissolvable and releasable from the backing layer after penetration into the skin.

36. The microneedle array of claim 35, wherein the biodegradable polymer is selected from polyvinylpyrrolidinone (PVP), polyvinylalcohol (PVA), low molecular weight hyaluronic acid, optionally ultra-low molecular weight hyaluronic acid, and super-low molecular weight hyaluronic acid, silk fibroin, maltose, poly(vinyl methyl ether)-co-maleic anhydride (PVME/MA), chitin, carboxymethylcellulose and its sodium salt, chondroitin sulfate and its sodium salt, sodium alginate, hydroxypropylcellulose, hydroxypropylmethylcellulose, dextran, and combinations thereof.

37. The microneedle array of claim 35 or 36, wherein the microneedles each comprises about 50 μM to about 500 μM, about 75 μM to about 400 μM, about 100 μM to about 300 μM, or about 150 μM of the sensor dye, and about 50 μM to about 300 μM, about 75 μM to about 250 μM, about 80 μM to about 200 μM, or about 100 μM of the reference dye.

38. The microneedle array of any one of claims 35 to 37, wherein the microneedles each comprises the sensor dye and reference dye at a ratio of about 3 to about 2.

39. The microneedle array of any one of claims 35 to 38, wherein the backing layer is or comprises a biodegradable polymer selected from polyvinylpyrrolidinone (PVP), polyvinylalcohol (PVA), low molecular weight hyaluronic acid, optionally ultra-low molecular weight hyaluronic acid, and super-low molecular weight hyaluronic acid, silk fibroin, maltose, poly(vinyl methyl ether)-co-maleic anhydride (PVME/MA), chitin, carboxymethylcellulose and its sodium salt, chondroitin sulfate and its sodium salt, sodium alginate, hydroxypropylcellulose, hydroxypropylmethylcellulose, dextran, and combinations thereof.

40. The microneedle array of any one of claims 35 to 39, wherein the microneedles are of a length sufficient to penetrate a stratum corneum layer and to reach an epidermis layer, optionally a dermis layer, optionally wherein the microneedles have a length of from about 200 nm to about 1500 nm, about 250 nm to about 1200 nm, or about 300 nm to about 1000 nm.

41. Method of delivering a ratiometric detection composition to a subject comprising administering a microneedle array as defined in any one of claims 35 to 40 to a skin of the subject.

42. The method of claim 41 further comprising removing the backing layer from the skin after a desired period of time, optionally after approximately 1 to 10 minutes.

43. Method of detection of an analyte comprising
detecting in a subject that had been administered a ratiometric detection composition as defined in any one of claims 1 to 30:
a fluorescence level of the reference dye; and
a fluorescence level of the sensor dye; and
assessing a presence of the analyte, wherein a positive value of the fluorescence level of the sensor dye normalized to the fluorescence level of the reference dye is indicative of the presence of the analyte.

44. The method of claim 43, wherein the method is for ratiometric detection of a plurality of analytes, and the ratiometric detection composition comprises a plurality of sensor dyes.

45. Method of measurement of an analyte comprising
detecting in a subject that had been administered a ratiometric detection composition as defined in any one of claims 1 to 30:
a fluorescence level of the reference dye; and
a fluorescence level of the sensor dye; and
determining a quantity of the analyte, wherein a value of the fluorescence level of the sensor dye normalized to the fluorescence level of the reference dye is indicative of the quantity of the analyte.

46. The method of any one of claims 43 to 45, wherein the administering of the ratiometric detection composition comprises a method as defined in claim 41 or 42.

47. The method of claim 45, wherein the method is for ratiometric measurement of a plurality of analytes, and the ratiometric detection composition comprises a plurality of sensor dyes.

48. The method of any one of claims 43 to 47, wherein the detecting of the fluorescence level of the reference dye and the detecting of the fluorescence level of the sensor dye are carried out with an imaging device, optionally a portable and/or wearable imaging device.

49. Method for monitoring of an analyte comprising
a) detecting in a subject that had been administered a ratiometric detection composition as defined in any one of claims 1 to 30:
an initial fluorescence level of the reference dye at an initial time point, and
an initial fluorescence level of the sensor dye at the initial time point;
b) determining a quantity of the analyte at the initial time point, wherein a value of the initial fluorescence level of the sensor dye normalized to the initial fluorescence level of the reference dye is indicative of the quantity of the analyte at the initial time point;
c) detecting in the subject:
a subsequent fluorescence level of the reference dye at a subsequent time point, and
a subsequent fluorescence level of the sensor dye at the subsequent time point;
d) determining a quantity of the analyte at the subsequent time point, wherein a value of the subsequent fluorescence level of the sensor dye normalized to the subsequent fluorescence level of the reference dye is indicative of the quantity of the analyte at the subsequent time point; and
e) optionally repeating steps c) to d).

50. The method of claim 49, wherein the method is for ratiometric monitoring of a plurality of analytes, and the ratiometric detection composition comprises a plurality of sensor dyes, and wherein the detecting of fluorescence levels of the sensor dyes comprises detecting fluorescence levels of each of the sensor dyes.

51. Method of detection of ROS comprising
detecting in a subject that had been administered a ratiometric detection composition as defined in any one of claims 2 to 6, 26, and 27:
a fluorescence level of the reference dye; and
a fluorescence level of the sensor dye; and
assessing a presence of the ROS, wherein a positive value of the fluorescence level of the sensor dye normalized to the fluorescence level of the reference dye is indicative of the presence of the ROS.

52. Method of measurement of ROS comprising
detecting in a subject that had been administered a ratiometric detection composition as defined in any one of claims 2 to 6, 26, and 27:
a fluorescence level of the reference dye; and
a fluorescence level of the sensor dye;
determining a quantity of the ROS, wherein a value of the fluorescence level of the sensor dye normalized to the fluorescence level of the reference dye is indicative of the quantity of the ROS.

53. Method for monitoring of ROS, the method comprising
a) detecting in a subject that had been administered a ratiometric detection composition as defined in any one of claims 2 to 6, 26, and 27:
an initial fluorescence level of the reference dye at an initial time point; and
an initial fluorescence level of the sensor dye at the initial time point;
b) determining a quantity of the analyte at the initial time point, wherein a value of the initial fluorescence level of the sensor dye normalized to the initial fluorescence level of the reference dye is indicative of the quantity of the ROS at the initial time point;
c) detecting in the subject:
a subsequent fluorescence level of the reference dye at a subsequent time point; and
a subsequent fluorescence level of the sensor dye at the subsequent time point; and
d) determining a quantity of the analyte at the subsequent time point, wherein a value of the subsequent fluorescence level of the sensor dye normalized to the subsequent fluorescence level of the reference dye is indicative of the quantity of the ROS at the subsequent time point,
e) optionally repeating steps c) to d).

54. Method of measurement of pH, the method comprising detecting in a subject that had been administered a ratiometric detection composition as defined in any one of claims 8 to 10, and 28:
a fluorescence level of the reference dye; and
a fluorescence level of the sensor dye; and
determining a pH value, wherein a value of the fluorescence level of the sensor dye normalized to the fluorescence level of the reference dye is indicative of the pH value.

55. Method for monitoring of pH, the method comprising
a) detecting in a subject that had been administered a ratiometric detection composition as defined in any one of claims 8 to 10, and 28:
an initial fluorescence level of the reference dye at an initial time point; and
an initial fluorescence level of the sensor dye at the initial time point;
b) determining a pH value at the initial time point, wherein a value of the initial fluorescence level of the sensor dye normalized to the initial fluorescence level of the reference dye is indicative of the pH value at the initial time point;
c) detecting in the subject:
a subsequent fluorescence level of the reference dye at a subsequent time point; and
a subsequent fluorescence level of the sensor dye at the subsequent time point; and
d) determining a pH value at the subsequent time point, wherein a value of the subsequent fluorescence level of the sensor dye normalized to the subsequent fluorescence level of the reference dye is indicative of the pH value at the subsequent time point,
e) optionally repeating steps c) to d).

56. Method of detection of a cation, the method comprising
detecting in a subject that had been administered a ratiometric detection composition as defined in any one of claims 11 to 25 and 28:
a fluorescence level of the reference dye; and
a fluorescence level of the sensor dye;
assessing a presence of the cation, wherein a positive value of the fluorescence level of the sensor dye normalized to the fluorescence level of the reference dye is indicative of the presence of the cation.

57. Method of measurement of a cation, the method comprising
detecting in a subject that had been administered a ratiometric detection composition as defined in any one of claims 11 to 25, and 28:
a fluorescence level of the reference dye; and
a fluorescence level of the sensor dye;
determining a quantity of the cation, wherein a value of the fluorescence level of the sensor dye normalized to the fluorescence level of the reference dye is indicative of the quantity of the cation.

58. Method for monitoring of a cation, the method comprising
a) detecting in a subject that had been administered a ratiometric detection composition as defined in any one of claims 11 to 25, and 28:
an initial fluorescence level of the reference dye at an initial time point; and
an initial fluorescence level of the sensor dye at the initial time point;
b) determining a quantity of the cation at the initial time point, wherein a value of the initial fluorescence level of the sensor dye normalized to the initial fluorescence level of the reference dye is indicative of the quantity of the cation at the initial time point;
c) detecting in the subject:
a subsequent fluorescence level of the reference dye at a subsequent time point; and
a subsequent fluorescence level of the sensor dye at the subsequent time point; and
d) determining a quantity of the cation at the subsequent time point, wherein a value of the subsequent fluorescence level of the sensor dye normalized to the subsequent fluorescence level of the reference dye is indicative of the quantity of the cation at the subsequent time point,
e) optionally repeating steps c) to d) and/or optionally comparing to the quantity of step b).

59. The method of any one of claims 56 to 58, wherein the cation is Li⁺ and the reference dye is as defined in claim 2 or 28 and the sensor dye is as defined in claim 12, or 13.

60. The method of any one of claims 56 to 58, wherein the cation is Na⁺ and the reference dye is as defined in claim 2 or 28 and the sensor dye is as defined in any one of claims 14, 15, and 20 to 22.

61. The method of any one of claims 56 to 58, wherein the cation is K⁺ and the reference dye is as defined in claim 2 or 26 and the sensor dye is as defined in claim 16, or 17.

62. The method of any one of claims 56 to 58, wherein the cation is H⁺ and the reference dye is as defined in claim 2 or 28 and the sensor dye is as defined in any one of claims 8 to 10, 18 and 19.

63. The method of any one of claims 43 to 62 further comprising administering topically in the subject the ratiometric detection composition, optionally the administering comprises the method of any one of claims 41 or 42.

64. A kit for ratiometric detection/measurement/monitoring of an analyte comprising
a microneedle array as defined in any one of claims 35 to 39;
an applicator device for applying the microneedle array, optionally a spring-loaded applicator device; and
optionally one or more of instructions for use, and disinfecting wipe.

65. The kit of claim 64 further comprising an imaging device, optionally a wearable/portable imaging device.
